(19) United States
(12) Patent Application Publication
(10) Pub. No.: US 2012/0270934 A1
(43) Pub. Date: Oct. 25, 2012

(54) PROSTACYCLIN DERIVATIVES

(75) Inventors: Craig E. Masse, Cambridge, MA (US); Scott L. Harbeson, Cambridge, MA (US); Roger D. Tung, Lexington, MA (US)

(73) Assignee: CoNCERT Pharmaceuticals, Inc.

(21) Appl. No.: 13/381,419

(22) PCT Filed: Jul. 2, 2010
(86) PCT No.: PCT/US10/40891

§ 371 Date: Jun. 5, 2012

Related U.S. Application Data
(60) Provisional application No. 61/222,955, filed on Jul. 3, 2009.

(51) Int. Cl. A61K 31/5578; C07D 317/40; A61K 31/5585; A61P 9/00; C07C 69/73; A61P 9/12; A61P 13/12; A61P 35/00; C07C 69/732; C07C 311/51

(52) U.S. Cl. 514/467; 560/119; 514/510; 560/8; 549/229; 564/98; 514/601

(57) ABSTRACT

This invention relates to novel prostacyclin derivatives and acceptable salts thereof. The invention also provides compositions comprising a compound of this invention and the use of such compositions in methods of treating diseases and conditions beneficially treated by prostacyclin, and in particular those diseases and conditions beneficially treated by dilators of systemic and pulmonary arterial vascular beds or by platelet aggregation inhibitors.

### RELATED APPLICATION

This application claims the benefit of U.S. Provisional Application No. 61/222,955, filed on Jul. 3, 2009, the contents of which are incorporated herein by reference in its entirety.

### TECHNICAL FIELD OF THE INVENTION

This invention relates to novel prostacyclin derivatives and their pharmaceutically acceptable salts. The invention also provides compositions comprising a compound of this invention and the use of such compositions in methods of treating diseases and conditions beneficially treated by prostacyclin, and in particular those diseases and conditions beneficially treated by dilators of systemic and pulmonary arterial vascular beds or by platelet aggregation inhibitors.

### BACKGROUND OF THE INVENTION

Many current medicines suffer from poor absorption, distribution, metabolism and/or excretion (ADME) properties that prevent their wider use or limit their use in certain indications. Poor ADME properties are also a major reason for the failure of drug candidates in clinical trials. While formulation technologies and prodrug strategies can be employed in some cases to improve certain ADME properties, these approaches often fail to address the underlying ADME problems that exist for many drugs and drug candidates. One such problem is rapid metabolism that causes a number of drugs, which otherwise would be highly effective in treating a disease, to be cleared too rapidly from the body. A possible solution to rapid drug clearance is frequent or high dosing to attain a sufficiently high plasma level of drug. This, however, introduces a number of potential treatment problems such as poor patient compliance with the dosing regimen, side effects that become more acute with higher doses, and increased cost of treatment. A rapidly metabolized drug may also expose patients to undesirable toxic or reactive metabolites.

Another ADME limitation that affects many medicines is the formation of toxic or biologically reactive metabolites. As a result, some patients receiving the drug may experience toxicities, or the safe dosing of such drugs may be limited such that patients receive a suboptimal amount of the active agent. In certain cases, modifying dosing intervals or formulation approaches can help to reduce clinical adverse effects, but often the formation of such undesirable metabolites is intrinsic to the metabolism of the compound.

In some select cases, a metabolic inhibitor will be co-administered with a drug that is cleared too rapidly. Such is the case with the protease inhibitor class of drugs that are used to treat HIV infection. The FDA recommends that these drugs be co-dosed with ritonavir, an inhibitor of cytochrome P450 enzyme 3A4 (CYP3A4), the enzyme typically responsible for their metabolism (see Kempf, D. J. et al., Antimicrobial agents and chemotherapy, 1997, 41(3): 654-60). Ritonavir, however, causes adverse effects and adds to the pill burden for HIV patients who must already take a combination of different drugs. Similarly, the CYP2D6 inhibitor quinidine has been added to dextromethorphan for the purpose of reducing rapid CYP2D6 metabolism of dextromethorphan in a treatment of pseudobulbar affect. Quinidine, however, has unwanted side effects that greatly limit its use in potential combination therapy (see Wang, L et al., Clinical Pharmacology and Therapeutics, 1994, 56(6 Pt 1): 659-67; and FDA label for quinidine at www.accessdata.fda.gov).

In general, combining drugs with cytochrome P450 inhibitors is not a satisfactory strategy for decreasing drug clearance. The inhibition of a CYP enzyme's activity can affect the metabolism and clearance of other drugs metabolized by that same enzyme. CYP inhibition can cause other drugs to accumulate in the body to toxic levels.

A potentially attractive strategy for improving a drug's metabolic properties is deuterium modification. In this approach, one attempts to slow the CYP-mediated metabolism of a drug or to reduce the formation of undesirable metabolites by replacing one or more hydrogen atoms with deuterium atoms. Deuterium is a safe, stable, non-radioactive isotope of hydrogen. Compared to hydrogen, deuterium forms stronger bonds with carbon. In select cases, the increased bond strength imparted by deuterium can positively impact the ADME properties of a drug, creating the potential for improved drug efficacy, safety, and/or tolerability. At the same time, because the size and shape of deuterium are essentially identical to those of hydrogen, replacement of hydrogen by deuterium would not be expected to affect the biochemical potency and selectivity of the drug as compared to the original chemical entity that contains only hydrogen.

Over the past 35 years, the effects of deuterium substitution on the rate of metabolism have been reported for a very small percentage of approved drugs (see, e.g., Blake, M I et al, J Pharm Sci, 1975, 64:367-91; Foster, A B, Adv Drug Res 1985, 14:1-40 (“Foster”); Kushner, D J et al, Can J Physiol Pharmacol 1999, 79-88; Fisher, M B et al, Curr Opin Drug Discov Devel, 2006, 9:101-09 (“Fisher”)). The results have been variable and unpredictable. For some compounds deuteration caused decreased metabolic clearance in vivo. For others, there was no change in metabolism. Still others demonstrated increased metabolic clearance. The variability in deuterium effects has also led experts to question or dismiss deuterium modification as a viable drug design strategy for inhibiting adverse metabolism (see Foster at p. 35 and Fisher at p. 101).

The effects of deuterium modification on a drug's metabolic properties are not predictable even when deuterium atoms are incorporated at known sites of metabolism. Only by actually preparing and testing a deuterated drug can one determine if and how the rate of metabolism will differ from that of its non-deuterated counterpart. See, for example, Fukuto et al. (J. Med. Chem. 1991, 34, 2871-76). Many drugs have multiple sites where metabolism is possible. The site(s) where deuterium substitution is required and the extent of deuteration necessary to see an effect on metabolism, if any, will be different for each drug.

Iloprost is a synthetic analogue of prostacyclin PGI2 and is described in U.S. Pat. No. 4,692,464. Iloprost is known by the chemical names (E)-(3aS,4R,5R,6aS)-hexahydro-5-4-[(E)-(3S,4RS)-3-hydroxy-4-methyl-1-octen-6-ynyl]-Δ^{2(1H),Δ}-pentalenevaleric acid; and 5-[(E)-(1S,5S,6R,7R)-7-hydroxy-6-[(E)-(3S,4RS)-3-hydroxy-4-methyl-1-octen-6-inyl]-bi-cyclo[3.3.0]octan-3-ylidene)pentanoic acid.

Iloprost is known to have in vitro pharmacological effects on inhibiting platelet aggregation and platelet adhesion. It is also known to cause dilation of arterioles and venules, and has been shown to reduce vascular permeability caused by mediators such as serotonin or histamine. Iloprost has also been shown to lower pulmonary arterial pressure in animal models of pulmonary hypertension. Its ability to inhibit pulmonary vasoconstriction and reduce pulmonary vascular resistance together with its platelet anti-aggregation and antithrombotic activity are factors that favor its use in the therapeutic treatment of pulmonary arterial hypertension. Such use has been approved in the United States using an inhalable formulation of iloprost.

Despite its efficacy, and because of its short half-life, iloprost must be administered 6 to 9 times per day, not more than once every 2 hours. This high frequency of administration can lead to problems with compliance such as missed dosages, and overdosing when compensating for missed dosages. Additionally, the patient does not experience adequate therapeutic coverage during sleep. More common side effects of iloprost include abnormal lab test; back pain; blurred vision, confusion, dizziness, faintness, or lightheadedness when getting up from a lying or sitting position suddenly; chills; cough increased; coughing or spitting up blood; diarrhea; difficulty opening the mouth; feeling of warmth; fever; general feeling of discomfort or illness; headache; joint pain; lockjaw; loss of appetite; muscle aches and pains; muscle cramps; muscle spasms, especially of neck and back; nausea; redness of the face, neck, arms and occasionally, upper chest; runny nose; shivering; sore throat; sweating; trouble sleeping; sleeplessness; unable to sleep; unusual tiredness or weakness; and vomiting. These side effects may be attributable to one or more of the metabolites of iloprost and/or overdosing due to poor compliance with the high number of dosages required on a daily basis.

Thus, despite the beneficial activities of iloprost, there is a continuing need for new and improved compounds to treat the aforementioned diseases and conditions.

### DEFINITIONS

The term “treat” means decrease, suppress, attenuate, diminish, arrest, or stabilize the development or progression of a disease (e.g., a disease or disorder delineated herein).

By “disease” is meant any condition or disorder that damages or interferes with the normal function of a cell, tissue, or organ.

The term “alkyl” refers to a monovalent saturated hydrocarbon group. C₁-C₂₀ alkyl is an alkyl having from 1 to 20 carbon atoms and includes, for example, C₁-C₁₄ alkyl, C₁-C₁₀ alkyl, and C₁-C₆ alkyl. An alkyl may be linear or branched. Examples of alkyl groups include methyl, ethyl, n-propyl, isopropyl, n-butyl, isobutyl, sec-butyl, t-butyl, n-pentyl, isopentyl, neopentyl and n-hexyl.

The term “C₁-C₂₀ linear alkyl” refers to an alkyl group of the formula CH₃—(CH₂)ₘ— where m is an integer from 0 to 19. Examples of C₁-C₂₀ linear alkyl include C₁-C₁₂ linear alkyl, wherein m is an integer between 0 and 11, and C₁-C₆ linear alkyl, wherein m is an integer between 0 and 5. More particular examples of C₁-C₂₀ linear alkyl groups include methyl, ethyl, n-propyl, n-butyl and n-pentyl.

The term “C₁-C₂₀ branched alkyl” refers to an alkyl group in which at least one carbon is bonded to at least three other carbon atoms. Examples of C₁-C₂₀ branched alkyl include C₁-C₁₂ branched alkyl and C₁-C₆ branched alkyl. More particular examples of C₁-C₂₀ branched alkyl groups include, isopropyl, isobutyl, sec-butyl, t-butyl, isopentyl, neopentyl, 2-methylpentyl and 3-methylpentyl.

The term “cycloalkyl” refers to a monocyclic, bicyclic, or tricyclic monovalent saturated hydrocarbon ring system. The term “C₃-C₁₄ cycloalkyl” refers to a cycloalkyl wherein the number of ring carbon atoms is from 3 to 14. Examples of C₃-C₁₄ cycloalkyl include C₃-C₁₀ cycloalkyl and C₃-C₆ cycloalkyl. Bicyclic and tricyclic ring systems include fused, bridged, and spirocyclic ring systems. More particular examples of cycloalkyl groups include, cyclopropyl, cyclobutyl, cyclopentyl, cyclohexyl, cycloheptyl, cis- and trans-decalinyl, norbornyl, adamantyl, and spiro[4.5]decanyl.

The term “carbocyclic ring” refers to a monocyclic, bicyclic, or tricyclic hydrocarbon ring system, which may be saturated or unsaturated. The term “C₃-C₁₄ carbocyclic ring” refers to a carbocyclic ring wherein the number of ring carbon atoms is from 3 to 14. Examples of C₃-C₁₄ carbocyclic ring include C₃-C₁₀ carbocyclic ring and C₃-C₆ carbocyclic ring.

The term “heterocycloalkyl” refers to a monocyclic, bicyclic, or tricyclic monovalent saturated ring system wherein from 1 to 4 ring atoms are heteroatoms independently selected from the group consisting of O, N and S. The term “3 to 14-membered heterocycloalkyl” refers to a heterocycloalkyl wherein the number of ring atoms is from 3 to 14. Examples of 3 to 14-membered heterocycloalkyl include 3 to 10-membered heterocycloalkyl and 3 to 6-membered heterocycloalkyl. Bicyclic and tricyclic ring systems include fused, bridged, and spirocyclic ring systems. More particular examples of heterocycloalkyl groups include azepanyl, azetidinyl, aziridinyl, imidazolidinyl, morpholinyl, oxazolidinyl, oxazolidinyl, piperazinyl, piperidinyl, pyrazolidinyl, pyrrolidinyl, quinuclidinyl, tetrahydrofuranyl, thiomorpholinyl, and 4-methyl-1,3-dioxol-2-onyl.

The term “aryl” refers to a monovalent aromatic carbocyclic ring system, which may be a monocyclic, fused bicyclic, or fused tricyclic ring system. The term “C₆-C₁₄ aryl” refers to an aryl having from 6 to 14 ring carbon atoms. An example of C₆-C₁₄ aryl is C₆-C₁₀ aryl. More particular examples of aryl groups include phenyl, naphthyl, anthracyl, and phenanthryl.

The term “heteroaryl” refers to a monovalent aromatic ring system wherein from 1 to 4 ring atoms are heteroatoms independently selected from the group consisting of O, N and S, and having from 5 to 14 ring atoms. The ring system may be a monocyclic, fused bicyclic, or fused tricyclic ring system. The term “5 to 14-membered heteroaryl” refers to a heteroaryl wherein the number of ring atoms is from 5 to 14. Examples of 5 to 14-membered heteroaryl include 5 to 10-membered heteroaryl and 5 to 6-membered heteroaryl. More particular examples of heteroaryl groups include furanyl, furazanyl, homopiperazinyl, imidazolinyl, isothiazolyl, isoxazolyl, oxadiazolyl, oxazolyl, pyrimidinyl, phenanthridinyl, pyranyl, pyrazinyl, pyrazolinyl, pyrazolyl, pyridazinyl, pyridooxazolyl, pyridoimidazolyl, pyridothiazolyl, pyridinyl, pyrimidinyl, pyrrolinyl, thiadiazinyl, thiadiazolyl, thienyl, thienothiazolyl, thienooxazolyl, thienoimidazolyl, thiophenyl, triazinyl, and triazolyl.

It will be recognized that some variation of natural isotopic abundance occurs in a synthesized compound depending upon the origin of chemical materials used in the synthesis. Thus, a preparation of iloprost will inherently contain small amounts of deuterated isotopologues. The concentration of naturally abundant stable hydrogen and carbon isotopes, notwithstanding this variation, is small and immaterial with respect to the degree of stable isotopic substitution of compounds of this invention. See, for instance, Wada, E et al., Seikagaku, 1994, 66:15; Gannes, L Z et al., Comp Biochem Physiol Mol Integr Physiol, 1998, 119:725.

In the compounds of this invention any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Also unless otherwise stated, when a position is designated specifically as “D” or “deuterium”, the position is understood to have deuterium at an abundance that is at least 3340 times greater than the natural abundance of deuterium, which is 0.015% (i.e., at least 50.1% incorporation of deuterium).

The term “isotopic enrichment factor” as used herein means the ratio between the isotopic abundance and the natural abundance of a specified isotope.

In other embodiments, a compound of this invention has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

The term “isotopologue” refers to species in which the chemical structure differs from a specific compound of this invention only in the isotopic composition of their molecules or ions.

The term “compound,” when referring to a compound of this invention, refers to a collection of molecules having an identical chemical structure, except that there may be isotopic variation among the constituent atoms of the molecules. Thus, it will be clear to those of skill in the art that a compound represented by a particular chemical structure containing indicated deuterium atoms will also contain lesser amounts of isotopologues having hydrogen atoms at one or more of the designated deuterium positions in that structure. The relative amount of such isotopologues in a compound of this invention will depend upon a number of factors including the isotopic purity of deuterated reagents used to make the compound and the efficiency of incorporation of deuterium in the various synthesis steps used to prepare the compound. However, as set forth above the relative amount of such isotopologues in toto will be less than 49.9% of the compound. In other embodiments, the relative amount of such isotopologues in toto will be less than 47.5%, less than 40%, less than 32.5%, less than 25%, less than 17.5%, less than 10%, less than 5%, less than 3%, less than 1%, or less than 0.5% of the compound.

The invention also provides salts of the compounds of the invention.

A salt of a compound of this invention is formed between an acid and a basic group of the compound, such as an amino functional group, or a base and an acidic group of the compound, such as a carboxyl functional group. According to another preferred embodiment, the compound is a pharmaceutically acceptable acid addition salt.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this invention. A “pharmaceutically acceptable counterion” is an ionic portion of a salt that is not toxic when released from the salt upon administration to a recipient.

Acids commonly employed to form pharmaceutically acceptable salts include inorganic acids such as hydrogen bisulfide, hydrochloric, hydrobromic, hydroiodic, sulfuric and phosphoric acid, as well as organic acids such as para-toluenesulfonic, salicylic, tartaric, bitartaric, ascorbic, maleic, besylic, fumaric, gluconic, glucuronic, formic, glutamic, methanesulfonic, ethanesulfonic, benzenesulfonic, lactic, oxalic, para-bromophenylsulfonic, carbonic, succinic, citric, benzoic and acetic acid, and related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylenesulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and the like salts. Preferred pharmaceutically acceptable acid addition salts include those formed with mineral acids such as hydrochloric acid and hydrobromic acid, and especially those formed with organic acids such as maleic acid.

The compounds of the present invention contain asymmetric carbon atoms at the 3 and 4 positions of the octen-6-ynyl side chain. The stereochemistry at the 3-position is S, which is the stereochemistry required for activity. As such, a compound of this invention can exist as the individual 3S,4S or 3S,4R diastereoisomers, as well a mixture of those two diastereoisomers. Accordingly, a compound of the present invention will include not only a stereoisomeric mixture, but also individual respective stereoisomers substantially free from other stereoisomers. The term “substantially free of other stereoisomers” as used herein means less than 25% of other stereoisomers, preferably less than 10% of other stereoisomers, more preferably less than 5% of other stereoisomers and most preferably less than 2% of other stereoisomers are present. Methods of obtaining or synthesizing diastereomers are well known in the art and may be applied as practicable to final compounds or to starting material or intermediates. Other embodiments are those wherein the compound is an isolated compound.

The term “stable compounds”, as used herein, refers to compounds which possess stability sufficient to allow manufacture and which maintain the integrity of the compound for a sufficient period of time to be useful for the purposes detailed herein (e.g., formulation into therapeutic products, intermediates for use in production of therapeutic compounds, isolatable or storable intermediate compounds, treating a disease or condition responsive to therapeutic agents).

“D” and “d” both refer to deuterium. “Stereoisomer” refers to both enantiomers and diastereomers. “Tert” and “t-” each refer to tertiary.

Throughout this specification, reference to “each Y” includes, independently, all “Y” groups (e.g., Y^{1a}, Y^{1b}, Y^{2a} and Y^{2b}) and reference to “each Z” includes, independently, all “Z” groups (e.g., Z^{1a} and Z^{1b}), where applicable.

### Therapeutic Compounds

The present invention provides a compound of Formula A:

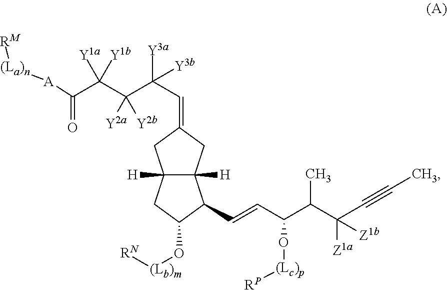

or a pharmaceutically acceptable salt thereof, wherein:

each Y is independently selected from hydrogen and deuterium;

each Z is independently selected from hydrogen, deuterium and fluorine;

Lₐ is

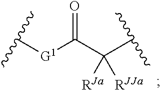

n=0 or 1;

R^{Ja} is C₁-C₂₀ alkyl or C₆-C₁₄ aryl, wherein R^{Ja} is optionally substituted with one or more J;

R^{JJa} is H or C₁-C₆ alkyl; or

R^{Ja} and R^{JJa}, taken together with the carbon atom to which they are both attached, form a C₃-C₁₀ carbocyclic ring;

L_{b} is

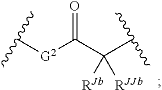

m=0 or 1;

R^{Jb} is C₁-C₂₀ alkyl or C₆-C₁₄ aryl, wherein R^{Jb} is optionally substituted with one or more J;

R^{JJb} is H or C₁-C₆ alkyl; or

R^{Jb} and R^{JJb}, taken together with the carbon atom to which they are both attached, form a C₃-C₁₀ carbocyclic ring;

L_{c} is

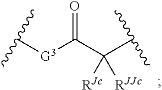

p=0 or 1;

R^{Jc} is C₁-C₂₀ alkyl or C₆-C₁₄ aryl, wherein R^{Jc} is optionally substituted with one or more J;

R^{JJc} is H or C₁-C₆ alkyl; or

R^{Jc} and R^{JJc}, taken together with the carbon atom to which they are both attached, form a C₃-C₁₀ carbocyclic ring;

each J is independently halogen, C₁-C₆ alkyl, hydroxyl, O—(C₁-C₆ alkyl), NH₂, NH(C₁-C₆ alkyl), N(C₁-C₆ alkyl)₂, NHC(═NH)NH₂, SH, SCH₃, CONH₂, COOH, CO₂(C₁-C₆ alkyl), C₆-C₁₀ aryl, or 5-14-membered heteroaryl, provided that if R^{Ja} is C₁-C₂₀ alkyl then J is not halogen;

R^{M} is C₁-C₂₀ linear alkyl, C₁-C₂₀ branched alkyl, C₆-C₁₄ aryl, 5-14-membered heteroaryl, C₃-C₁₄ cycloalkyl, or 3-14-membered heterocycloalkyl, wherein R^{M}, R^{N} and R^{P} are each optionally substituted with one or more M;

R^{N} and R^{P} are each independently selected from H, (CO)_{z}—C₁-C₂₀ linear alkyl, (CO)_{z}—C₁-C₂₀ branched alkyl, C₆-C₁₄ aryl, 5-14-membered heteroaryl, C₃-C₁₄ cycloalkyl, or 3-14-membered heterocycloalkyl, wherein when R^{N} is not H, R^{N} is optionally substituted with one or more M; and wherein when R^{P} is not H, R^{P} is optionally substituted with one or more M;

z is 0 or 1;

each M is independently C₁-C₆ alkyl, C₆-C₁₀ aryl, or 3-14-membered heterocycloalkyl wherein the M alkyl, aryl and/or heterocycloalkyl is optionally substituted with one or more W, wherein each W is independently C₁-C₆ alkyl or ═O, wherein if R^{M}, R^{N} or R^{P} is C₁-C₂₀ linear alkyl, then one or more methylene groups in R^{M}, R^{N} or R^{P} are optionally replaced with oxygen, with the proviso that each oxygen replacement is separated from any other oxygen replacement by at least two methylene groups;

A is —O—, —NH—, or —NH—SO₂—, with the proviso that, if A is —NH—SO₂—, then n is 0, R^{M} is bound to the —SO₂— of A, and R^{M} is C₁-C₂₀ branched alkyl optionally substituted with halogen, C₁-C₂₀ linear alkyl optionally substituted with halogen, C₆-C₁₀ aryl, 5-10-membered heteroaryl, or 3-10-membered heterocycloalkyl, wherein in the R^{M} C₁-C₂₀ linear alkyl no methylene group is replaced with oxygen; and

G¹, G² and G³ are each independently selected from O, NH and CH₂.

For the purpose of clarity and unless otherwise specified, the recitation that A may be —NH—SO₂—, is intended to include both permutations of that moiety, i.e., —NH—SO₂— and —SO₂—NH—.

In one embodiment of a compound of Formula A, Y^{3a} and Y^{3b} are hydrogen; m is 0; p is 0; R^{N} and R^{P} are each H; and when Z^{1a} and Z^{1b} are each hydrogen, at least one of Y^{1a}, Y^{1b}, Y^{2a}, or Y^{2b} is deuterium, the compound being a compound of Formula I:

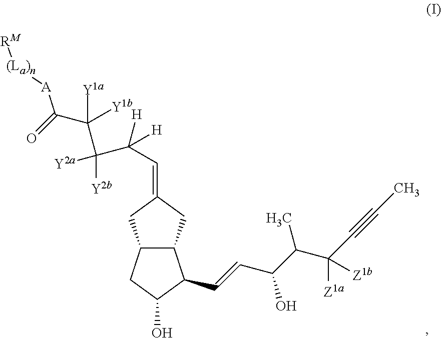

or a pharmaceutically acceptable salt thereof, wherein each Y, each Z; Lₐ, R^{M}, A, and n are as defined for Formula A.

In another embodiment of Formula A, m is 0; p is 0; R^{N} and R^{P} are each H; at least one Y or one Z is deuterium; and Z^{1a} and Z^{1b} are independently selected from hydrogen and deuterium, the compound being a compound of Formula II:

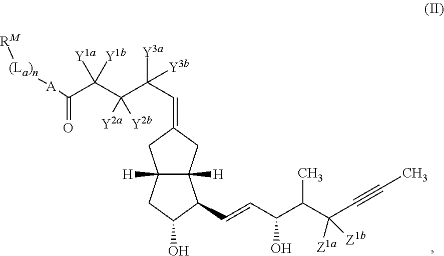

or a pharmaceutically acceptable salt thereof, wherein each Z is independently selected from hydrogen and deuterium; and each Y, Lₐ, R^{M}, A, and n are as defined for Formula A.

In one embodiment of Formula I or II, R^{M} is optionally substituted with one or two M, wherein M is as defined in Formula A.

In one embodiment of a compound of Formula I or II, A is —O—; and n is 0. A compound of Formula I in this embodiment has Formula I′:

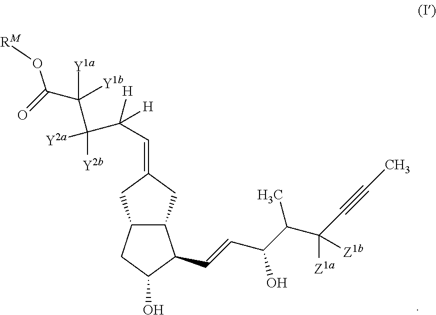

A compound of Formula II in this embodiment has Formula II′:

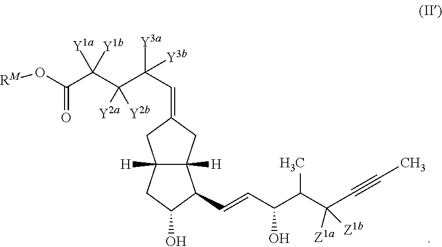

In one embodiment of the compound of Formula I′, or II′, R^{M} is C₁-C₂₀ linear alkyl or C₁-C₂₀ branched alkyl, wherein R^{M} is optionally substituted with one or more M, such as one or two M. In one aspect of this embodiment, R^{M} is methyl, ethyl, isopropyl, isobutyl, neopentyl, n-heptyl, 2-octyl, 3-methyl-1-butyl, or 3-methyl-2-butyl, each optionally substituted with one or more M, such as one or two M. In one aspect of this embodiment, each of the one or more M, such as one or two M, is C₆-C₁₀ aryl, such as phenyl. In this aspect, R^{M} may be, for example, methyl or ethyl. In one aspect of this embodiment, each of the one or more M, such as one or two M, is an independently selected 3-14-membered heterocycloalkyl optionally substituted with one or more W. In this aspect, R^{M} may be, for example, methyl, and one M may be, for example 4-methyl-1,3-dioxol-2-onyl.

In one embodiment of the compound of Formula I′ or II′, R^{M} is C₆-C₁₄ aryl, C₃-C₁₄ cycloalkyl, or 3 to 14-membered heterocycloalkyl, wherein R^{M} is optionally substituted with one or more M, such as one or two M. In one aspect of this embodiment, R^{M} is cyclohexyl. In one aspect of this embodiment, R^{M} is phenyl optionally substituted with one or more methyl. In one aspect of this embodiment, R^{M} is quinuclidinyl.

In one embodiment of the compound of Formula I′ or II′, R^{M} is C₁-C₂₀ linear alkyl wherein one or more methylene groups in R^{M} are replaced with oxygen. In one aspect of this embodiment, R^{M} is CH₃—(O—CH₂—CH₂)₃—.

In another embodiment of Formula I or II, A is —NH—SO₂—, wherein the S(O)₂ portion of A is bound to R^{M}; and n is 0. A compound of Formula I in this embodiment has Formula I″:

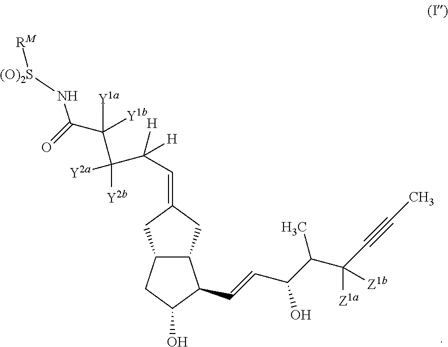

A compound of Formula II in this embodiment has Formula II″:

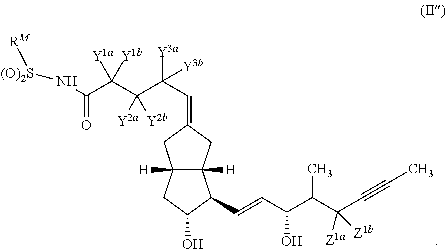

In one embodiment of the compound of Formula I″ or II″, R^{M} is C₁-C₆ branched alkyl optionally substituted with one or more M, such as one or two M, wherein each M is independently selected from halogen, C₁-C₆ linear alkyl optionally substituted with one or more halogen, phenyl, 5-10-membered heteroaryl, and 3-10-membered heterocycloalkyl. In one aspect of this embodiment, R^{M} is methyl optionally substituted with one or more M, such as one or two M, wherein each M is independently selected from fluorine, isopropyl, phenyl, 2-thiophenyl, 3-thiophenyl, or N-morpholinyl.

In another embodiment of Formula I or II, A is —O— or —NH—; and n is 1. A compound of Formula I in this embodiment has Formula I′″:

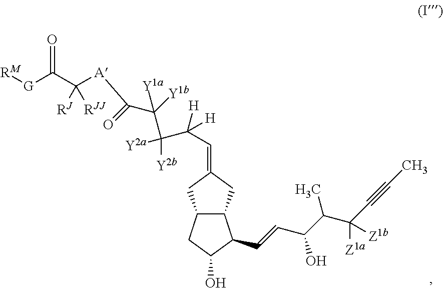

wherein A′ is —O— or —NH—. A compound of Formula II in this embodiment has Formula II′″:

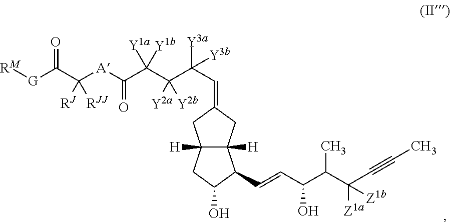

wherein A′ is —O— or —NH—.

In one embodiment of Formula I′″ or II′″, A′ is —O—. In one aspect of this embodiment, R^{M} is C₁-C₂₀ linear alkyl or C₁-C₂₀ branched alkyl wherein R^{M} is optionally substituted with one or more M such as one or two M. In this aspect, R^{M} may be, for example, ethyl or methyl. In one aspect of this embodiment, R^{Ja} is C₆-C₁₀ aryl, such as phenyl; and R^{JJa} is hydrogen. In one aspect of this embodiment, R^{Ja} is C₁-C₂₀ alkyl, such as methyl, isopropyl, or isobutyl; and R^{JJa} is hydrogen or C₁-C₆ alkyl such as methyl.

In one embodiment of the compound of any of Formula I, I′, I″, I′″, II, II′, II″, or II′″, R^{Ja} and R^{JJa}, taken together with the carbon to which they are both attached, form a C₃-C₁₀ carbocyclic ring. In one aspect of this embodiment, the carbocyclic ring is a C₃-C₈ carbocyclic ring, such as a C₃, C₄, C₅, or C₆ carbocyclic ring.

In one embodiment of the compound of any of Formula I, I′, I″, I′″, II, II′, II″, or II′″, Y^{1a} and Y^{1b} are the same. In one aspect of this embodiment, Y^{1a} and Y^{1b} are simultaneously deuterium.

In another embodiment of the compound of any of Formula I, I′, I″, I′″, II, II′, II″, or II′″, Y^{2a} and Y^{2b} are the same. In one aspect of this embodiment, Y^{2a} and Y^{2b} are simultaneously deuterium.

In one particular embodiment of the compound of Formula I, I′, I″, I′″, II, II′, II″, or II′″, Y^{1a}, Y^{1b}, Y^{2a}, and Y^{2b} are simultaneously deuterium.

In an embodiment of the compound of any of Formula I, I′, I″ or I′″, Z^{1a} and Z^{1b} are independently selected from deuterium or fluorine. In one aspect of this embodiment, Z^{1a} and Z^{1b} are the same. In a more specific aspect, Z^{1a} and Z^{1b} are simultaneously deuterium or simultaneously fluorine. In a still more specific aspect, Z^{1a} and Z^{1b} are simultaneously deuterium.

In an embodiment of the compound of any of Formula II, II′, II″, or II′″, Z^{1a} and Z^{1b} are the same. In one aspect of this embodiment, Z^{1a} and Z^{1b} are simultaneously deuterium. In another aspect of this embodiment, Y^{1a}, Y^{1b}, R^{2a}, and Y^{2b} are the same.

In one particular embodiment of Formula II, II′, II″, or II′″, Y^{1a}, Y^{1b}, Y^{2a}, and Y^{2b} are simultaneously hydrogen.

In one particular embodiment of Formula II, II′, II″, or II′″, Y^{3a} and Y^{3b} are simultaneously deuterium.

In one embodiment of the compound of Formula I or I′″, or a pharmaceutically acceptable salt thereof, R^{M}-(Lₐ)ₙ-A- is

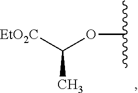

and Y^{1a}, Y^{1b}, Y^{2a}, Y^{2b}, Z^{1a} and Z^{1b} are as set forth in the following table:

TABLE 1a
Exemplary Embodiments of Formula I

| Cmpd | Y^{1a} | Y^{1b} | Y^{2a} | Y^{2b} | Z^{1a} | Z^{1b} |
|---|---|---|---|---|---|---|
| 200a | D | D | H | H | H | H |
| 201a | H | H | D | D | H | H |
| 202a | D | D | D | D | H | H |
| 203a | D | D | H | H | D | D |
| 204a | H | H | D | D | D | D |
| 205a | D | D | D | D | D | D |
| 206a | D | D | H | H | F | F |
| 207a | H | H | D | D | F | F |
| 208a | D | D | D | D | F | F |
| 209a | H | H | H | H | D | D |

In another embodiment of the compound of Formula I or I′″, or a pharmaceutically acceptable salt thereof, R^{M}-(Lₐ)ₙ-A- is (CH₃)₂CHO—, and Y^{1a}, Y^{1b}, Y^{2a}, Y^{2b}, Z^{1a} and Z^{1b} are as set forth in the following table:

TABLE 1b
Exemplary Embodiments of Formula I

| Cmpd | Y^{1a} | Y^{1b} | Y^{2a} | Y^{2b} | Z^{1a} | Z^{1b} |
|---|---|---|---|---|---|---|
| 200b | D | D | H | H | H | H |
| 201b | H | H | D | D | H | H |
| 202b | D | D | D | D | H | H |
| 203b | D | D | H | H | D | D |
| 204b | H | H | D | D | D | D |
| 205b | D | D | D | D | D | D |
| 206b | D | D | H | H | F | F |
| 207b | H | H | D | D | F | F |
| 208b | D | D | D | D | F | F |
| 209b | H | H | H | H | D | D |

In another embodiment of the compound of Formula I or I′, or a pharmaceutically acceptable salt thereof, n is 0; R^{M}-(Lₐ)ₙ-A- is CH₃CH₂O—; and Y^{1a}, Y^{1b}, Y^{2a}, Y^{2b}, Z^{1a} and Z^{1b} are as set forth in the following table:

TABLE 1c
Exemplary Embodiments of Formula I

| Cmpd | Y^{1a} | Y^{1b} | Y^{2a} | Y^{2b} | Z^{1a} | Z^{1b} |
|---|---|---|---|---|---|---|
| 200c | D | D | H | H | H | H |
| 201c | H | H | D | D | H | H |
| 202c | D | D | D | D | H | H |
| 203c | D | D | H | H | D | D |
| 204c | H | H | D | D | D | D |
| 205c | D | D | D | D | D | D |
| 206c | D | D | H | H | F | F |
| 207c | H | H | D | D | F | F |
| 208c | D | D | D | D | F | F |
| 209c | H | H | H | H | D | D |

In another embodiment of the compound of Formula II or II′″, or a pharmaceutically acceptable salt thereof, R^{M}-(Lₐ)ₙ-A- is

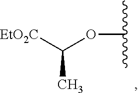

and Y^{1a}, Y^{1b}, Y^{2a}, Y^{2b}, Y^{3a}, Y^{3b}, Z^{1a} and Z^{1b} are as set forth in the following table:

TABLE 2a
Exemplary Embodiments of Formula II

| Cmpd | Y^{1a} | Y^{1b} | Y^{2a} | Y^{2b} | Y^{3a} | Y^{3b} | Z^{1a} | Z^{1b} |
|---|---|---|---|---|---|---|---|---|
| 210a | D | D | H | H | D | D | H | H |
| 211a | H | H | D | D | D | D | H | H |
| 212a | D | D | D | D | D | D | H | H |
| 213a | D | D | H | H | D | D | D | D |
| 214a | H | H | D | D | D | D | D | D |
| 215a | D | D | D | D | D | D | D | D |

In another embodiment of the compound of Formula II or II′, or a pharmaceutically acceptable salt thereof, n is 0; R^{M}-(Lₐ)ₙ-A- is (CH₃)₂CHO—; and Y^{1a}, Y^{1b}, Y^{2a}, Y^{2b}, Y^{3a}, Y^{3b}, Z^{1a} and Z^{1b} are as set forth in the following table:

TABLE 2b
Exemplary Embodiments of Formula II

| Cmpd | Y^{1a} | Y^{1b} | Y^{2a} | Y^{2b} | Y^{3a} | Y^{3b} | Z^{1a} | Z^{1b} |
|---|---|---|---|---|---|---|---|---|
| 210b | D | D | H | H | D | D | H | H |
| 211b | H | H | D | D | D | D | H | H |
| 212b | D | D | D | D | D | D | H | H |
| 213b | D | D | H | H | D | D | D | D |
| 214b | H | H | D | D | D | D | D | D |
| 215b | D | D | D | D | D | D | D | D |

In another embodiment of the compound of Formula II or II′″, or a pharmaceutically acceptable salt thereof, R^{M}-(Lₐ)ₙ-A- is CH₃CH₂O—, and Y^{1a}, Y^{1b}, Y^{2a}, Y^{2b}, Y^{3a}, Y^{3b}, Z^{1a} and Z^{1b} are as set forth in the following table:

TABLE 2c
Exemplary Embodiments of Formula II

| Cmpd | Y^{1a} | Y^{1b} | Y^{2a} | Y^{2b} | Y^{3a} | Y^{3b} | Z^{1a} | Z^{1b} |
|---|---|---|---|---|---|---|---|---|
| 210c | D | D | H | H | D | D | H | H |
| 211c | H | H | D | D | D | D | H | H |
| 212c | D | D | D | D | D | D | H | H |
| 213c | D | D | H | H | D | D | D | D |
| 214c | H | H | D | D | D | D | D | D |
| 215c | D | D | D | D | D | D | D | D |

In still another embodiment of Formula A, I or II, the group

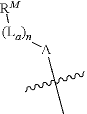

is selected from the group consisting of the following:

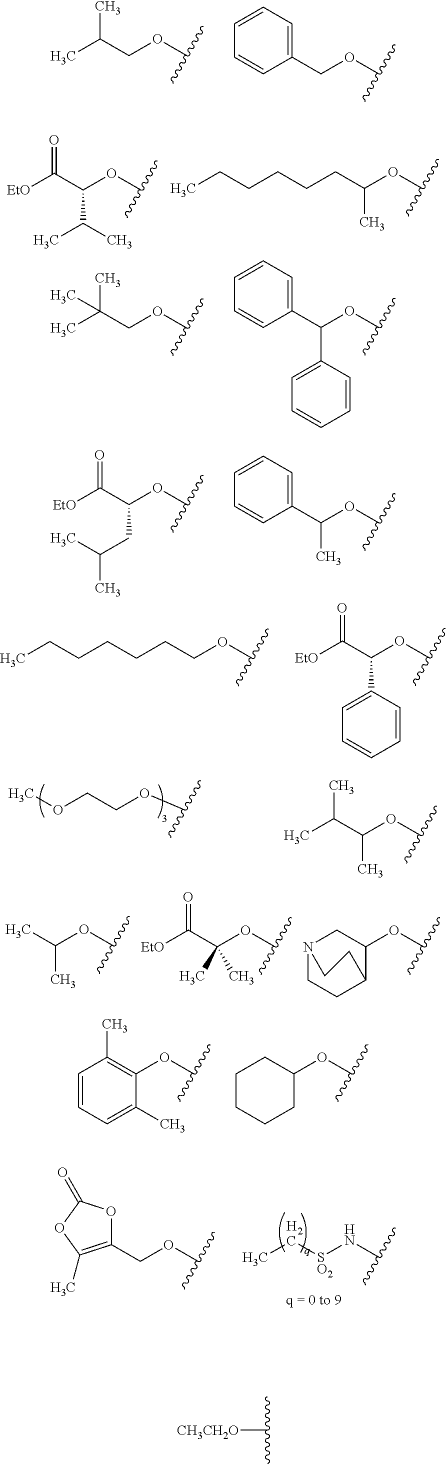

In one aspect of this embodiment, the compound is a compound of Formula I, or a pharmaceutically acceptable salt thereof, and Y^{1a}, Y^{1b}, Y^{2a}, Y^{2b}, Z^{1a} and Z^{1b} are as set forth in Table 3 below.

TABLE 3
Combinations of Y^{1a}, Y^{1b}, Y^{2a}, Y^{2b}, Z^{1a} and Z^{1b} Present in Compounds of

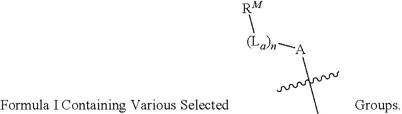

| | Y^{1a} | Y^{1b} | Y^{2a} | Y^{2b} | Z^{1a} | Z^{1b} |
|---|---|---|---|---|---|---|
| 1 | D | D | H | H | H | H |
| 2 | H | H | D | D | H | H |
| 3 | D | D | D | D | H | H |
| 4 | D | D | H | H | D | D |
| 5 | H | H | D | D | D | D |
| 6 | D | D | D | D | D | D |
| 7 | D | D | H | H | F | F |
| 8 | H | H | D | D | F | F |
| 9 | D | D | D | D | F | F |
| 10 | H | H | H | H | D | D |

or a pharmaceutically acceptable salt thereof. In another aspect of this embodiment, the compound is a compound of Formula II, or a pharmaceutically acceptable salt thereof, and Y^{1a}, Y^{1b}, Y^{2a}, Y^{2b}, Y^{3a}, Y^{3b}, Z^{1a} and Z^{1b} are as set forth in the Table 4, below.

TABLE 4
Combinations of Y^{1a}, Y^{1b}, Y^{2a}, Y^{2b}, Y^{3a}, Y^{3b}, Z^{1a} and Z^{1b} Present in

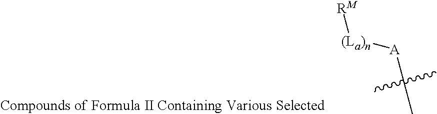

Groups.

| Y^{1a} | Y^{1b} | Y^{2a} | Y^{2b} | Y^{3a} | Y^{3b} | Z^{1a} | Z^{1b} |
|---|---|---|---|---|---|---|---|
| D | D | H | H | D | D | H | H |
| H | H | D | D | D | D | H | H |
| D | D | D | D | D | D | H | H |
| D | D | H | H | D | D | D | D |
| H | H | D | D | D | D | D | D |
| D | D | D | D | D | D | D | D |

In another set of embodiments, any atom not designated as deuterium in any of the embodiments of Formula I, I′, I″, I′″, II, II′, II″, or II′″ set forth above is present at its natural isotopic abundance.

The present invention also provides a compound of Formula A, wherein m is 0; p is 0; R^{N} and R^{P} are each hydrogen; and Y^{1a}, Y^{1b}, Y^{2a}, Y^{2b}, Y^{3a}, Y^{3b}, Z^{1a} and Z^{1b} are each hydrogen, the compound represented by Formula III:

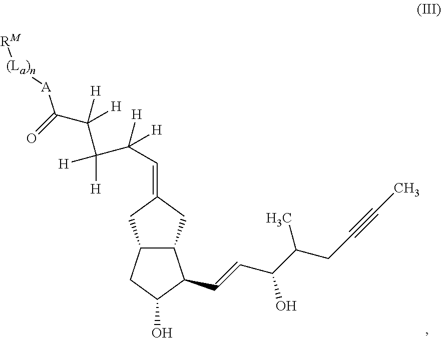

or a pharmaceutically acceptable salt thereof.

In one embodiment, the compound of Formula III is a compound wherein

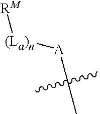

is selected from the group consisting of the following:

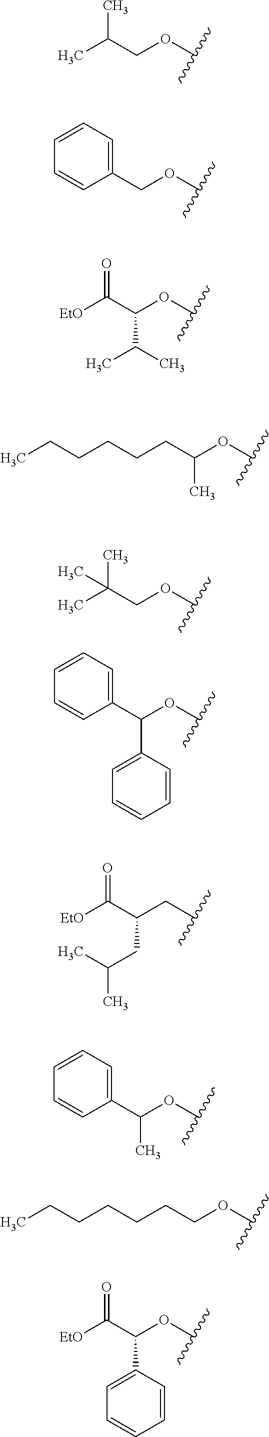

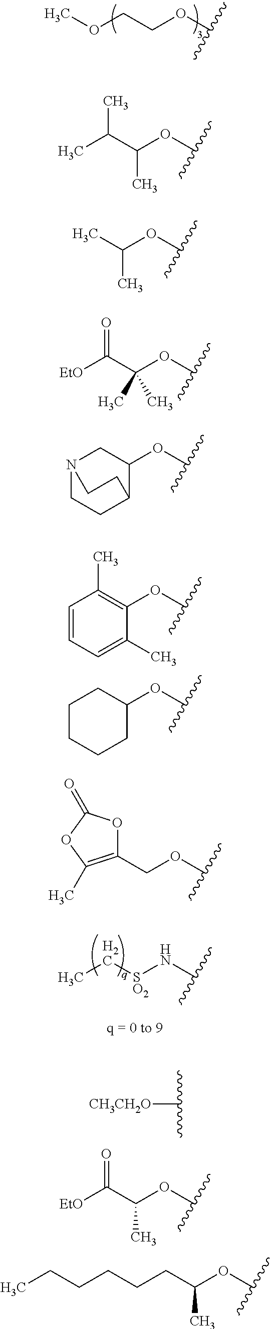

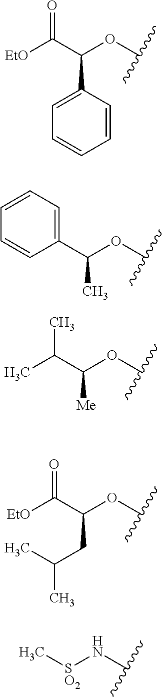

In one embodiment of the compound of Formula III,

n=1;

R^{Ja} is C₁-C₂₀ alkyl or C₆-C₁₄ aryl, wherein R^{Ja} is optionally substituted with one or more J;

and G¹ is O.

In an example of this embodiment, R^{Ja} is C₁-C₂₀ alkyl substituted with one or more phenyl. In a more particular example, R^{Ja} is C₁-C₂₀ alkyl substituted with one or more phenyl at the carbon bonded to —C(O)-G¹. For clarification, the carbon bonded to —C(O)-G¹ is indicated as C^{†} in the following substructure

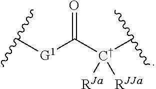

In one embodiment of the compound of Formula III,

n=0;

R^{M} is C₁-C₂₀ linear alkyl, C₆-C₁₄ aryl, C₃-C₁₄ cycloalkyl, or 3-14-membered heterocycloalkyl wherein R^{M} is optionally substituted with one or more M;

each M is independently C₁-C₆ alkyl or C₆-C₁₀ aryl optionally substituted with one or more W, wherein if R^{M} is C₁-C₂₀ linear alkyl, then up to three methylene groups in R^{M} are optionally replaced with oxygen, with the proviso that each oxygen replacement is separated from any other oxygen replacement by at least two methylene groups; and

A is —O— or —NH—SO₂—.

In an example of this embodiment, R^{M} is C₁-C₂₀ alkyl optionally substituted with one or more phenyl; and A is —O—. In a more particular example, R^{Ja} is C₁-C₂₀ alkyl substituted with one or more phenyl at the carbon bonded to A, wherein A is —O—. In another example of this embodiment, R^{M} is C₆-C₁₄ aryl optionally substituted with one or more C₁-C₄ alkyl; and A is —O—. In another example of this embodiment, R^{M} is 3-14-membered heterocycloalkyl optionally substituted with one or more C₁-C₄ alkyl; and A is —O—. In another example of this embodiment, R^{M} is C₃-C₁₄ cycloalkyl optionally substituted with one or more C₁-C₄ alkyl; and A is —O—. In another example of this embodiment, R^{M} is C₁-C₂₀ alkyl optionally substituted with one or more phenyl; and A is —NH—SO₂—.

In another set of embodiments, any atom not designated as deuterium in any of the embodiments of Formula III set forth above is present at its natural isotopic abundance

In one embodiment, the compound of Formula III is selected from the group consisting of the compounds of Table 5 and pharmaceutically acceptable salts thereof, wherein any atom not designated as deuterium in any of the compounds of Table 5 is present at its natural isotopic abundance:

TABLE 5
Compounds of Formula III Containing Various Selected
R^{M}—(Lₐ)ₙ—A Groups.

| Cmpd | R^{M}—(Lₐ)ₙ—A |
|---|---|
| 220 | 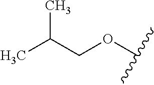 |
| 221 | 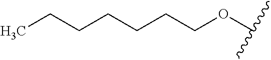 |
| 222 | 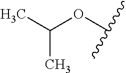 |
| 223 | 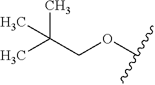 |
| 224 | 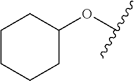 |
| 225 | 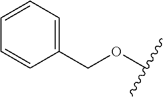 |
| 226 | 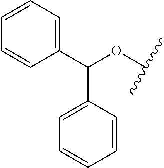 |
| 227 | 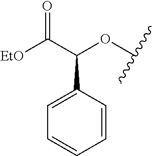 |
| 228 | 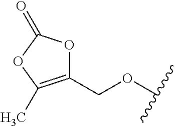 |
| 229 | 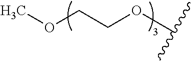 |
| 230 | 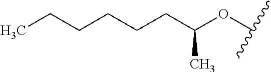 |
| 231 | 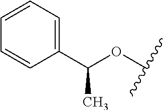 |
| 232 | 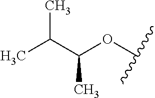 |
| 233 | 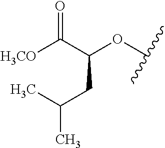 |
| 234 | 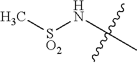 |
| 235 | 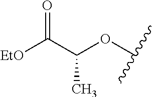 |
| 237 | 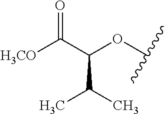 |

The present invention also provides a compound of Formula A, wherein m is 0; p is 0; R^{N} and R^{P} are each (CO)_{z}—C₁-C₆ linear alkyl wherein z is 1; and Y^{1a}, Y^{1b}, Y^{2a}, Y^{2b}, Y^{3a}, Y^{3b}, Z^{1a} and Z^{1b} are each hydrogen, the compound represented by Formula IV:

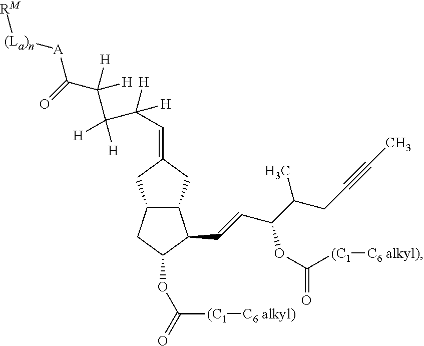

or a pharmaceutically acceptable salt thereof.

In one embodiment, the compound of Formula IV is a compound wherein

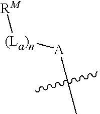

is selected from the group consisting of the following:

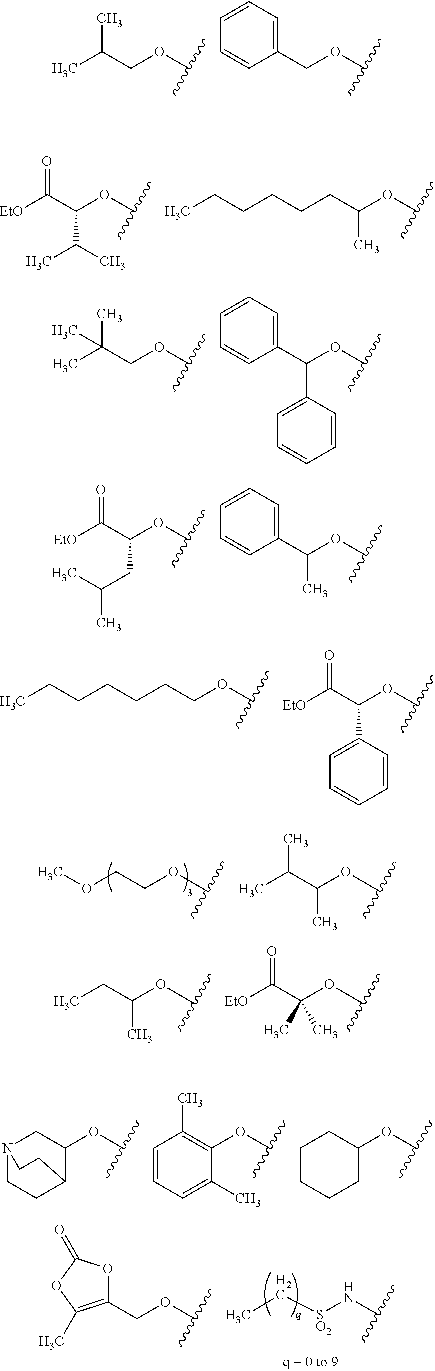

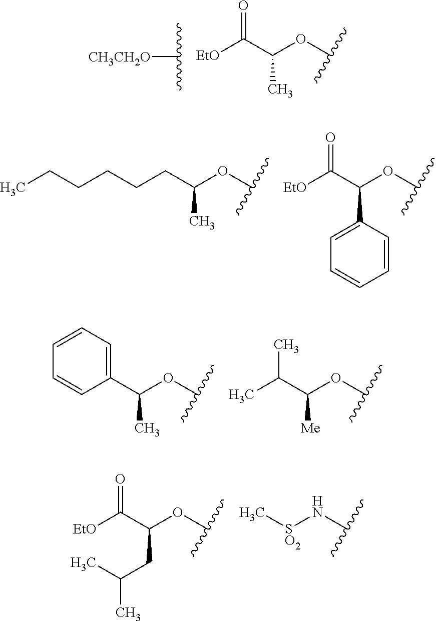

In another set of embodiments, any atom not designated as deuterium in any of the embodiments of Formula IV set forth above is present at its natural isotopic abundance

In one embodiment, the compound of Formula A is Compound 236 or a pharmaceutically acceptable salt thereof, wherein any atom not designated as deuterium is present at its natural isotopic abundance:

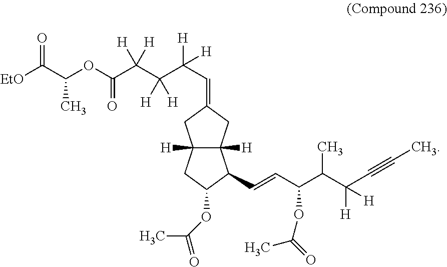

Compounds of Formula A where Y^{1a}, Y^{1b}, Y^{2a}, and Y^{2b} are simultaneously deuterium may be obtained as described below where the level of deuterium incorporation is at least 90% at each position designated as D. Such chemical structure modifications are particularly beneficial in improving the metabolic stability of the present compounds relative to iloprost. Accordingly, one embodiment of the invention relates to a deuterated iloprost where Y^{1a}, Y^{1b}, Y^{2a}, and Y^{2b} are simultaneously deuterium, preferably with at least 90% deuterium incorporation at each position. In such compounds, all other atoms may be at natural abundance or one or more hydrogen atoms may be optionally replaced by deuterium.

The metabolic stability that deuteration of the upper side chain confers on iloprost-based compounds, as described herein, can be applied to other iloprost compounds. For example, W. Skuballa et al., J. Med. Chem. 1986, 29(3): 313-315 reports certain iloprost analogues that have nearly identical profiles of action and potency. These authors modify iloprost in the bottom side chain by replacing the 13-14 double bond by a triple bond, introducing a further methyl at C-20 and preparing the (S)-diastereomer at C-16.

One embodiment provides compounds of Formula A where Y^{3a} and Y^{3b} are the same and Z^{1a} and Z^{1b} are the same. When Z^{1a} and Z^{1b} are the same, there are two chiral centers in the oct-1-en-6-ynyl chain. The chiral center bearing the CH₃ may be predominantly in the (S) configuration, or a 50/50 mixture of (R) and (S) stereochemistry, or predominantly in the (R) configuration, such as, for example, in a 60:40 (R)/(S) ratio, or such as, for example, in a 53:47 (R)/(S) ratio.

The synthesis of compounds of Formula A, or any of the other Formulae of the invention can be readily carried out by synthetic chemists of ordinary skill with references to the schemes below, which illustrate how the present compounds may be prepared, as well as to the Examples below.

A convenient method for producing compounds of Formula I is exemplified according to Schemes 1a, 1b and 1c:

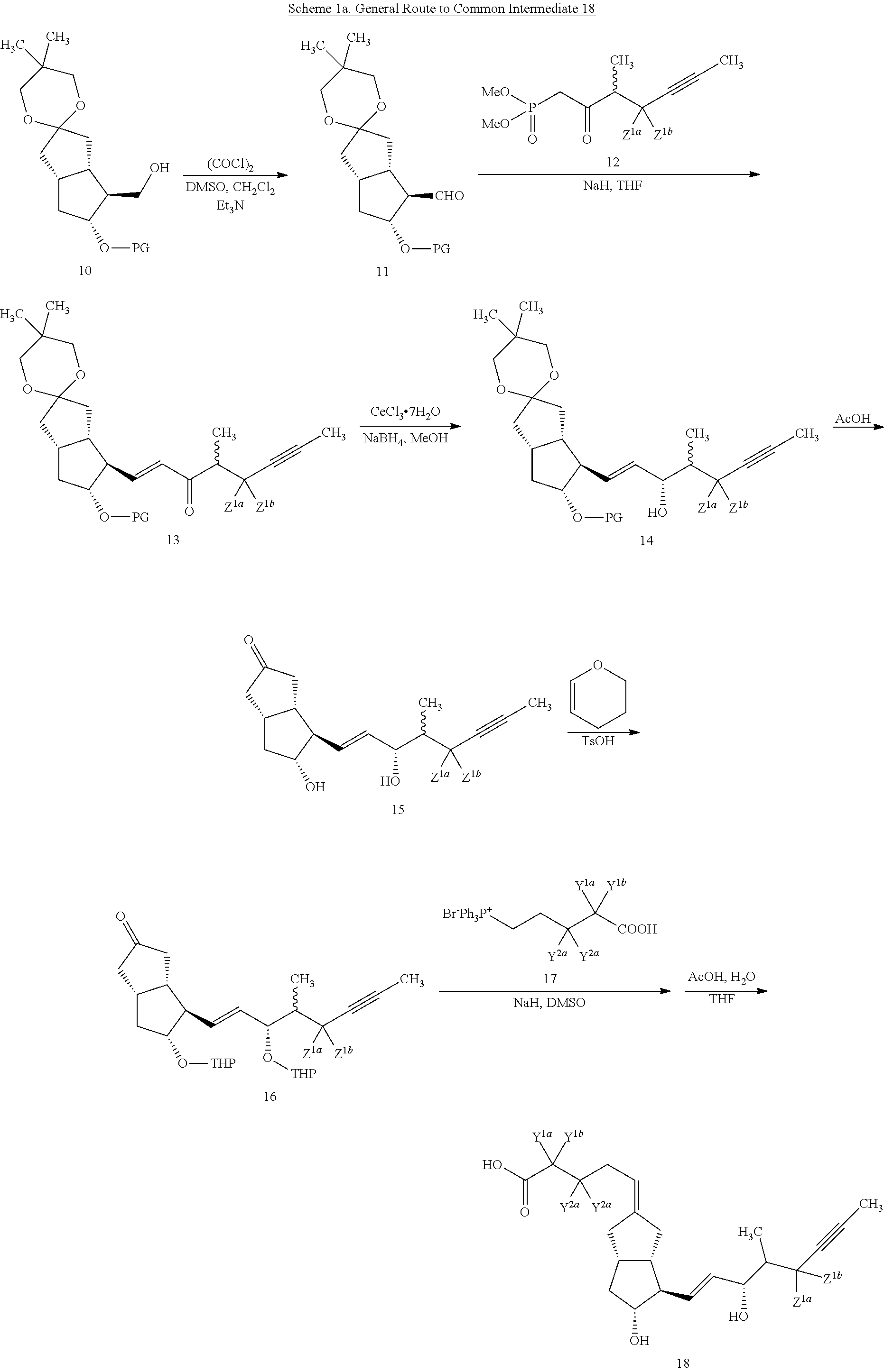

The synthesis of iloprost is described in U.S. Pat. No. 5,200,530 and the references cited therein.

“PG” represents a protecting group, such as a silyl ether protecting group, examples of which include t-butyldimethylsilyl, dimethylphenylsilyl or dimethylthexylsilyl (see, for example, T. W. Greene and P. G. M. Wuts, Protective Groups in Organic Synthesis, 3^{rd} Ed., John Wiley and Sons (1999)). Scheme 1a shows how the general route to iloprost may be adapted to provide a compound of Formula Ia. The primary alcohol 10 is oxidized to the aldehyde by first reacting with oxalyl chloride and DMSO in DCM (dichloromethane, methylene chloride, CH₂Cl₂) followed by addition of triethylamine. The desired aldehyde 11 is then reacted with the dimethylphosphonate 12 in THF using sodium hydride as the base to provide 13. The ketone is reduced to the alcohol 14 in methanol using sodium borohydride and cerium chloride heptahydrate. Excess borohydride reagent is quenched with acetone to yield 14. The ketal and silyl ether protecting groups are removed by treatment with acid and tetrabutylammonium fluoride, respectively, to yield 15. The secondary alcohols are protected as the THP ethers by reaction with dihydropyran with toluene sulfonic acid as a catalyst to yield 16. The ketone is then reacted with the ylide of the appropriately substituted triphenyl pentanoic acid in DMSO with sodium hydride as base. Subsequent removal of the THP protecting groups with mild acid yields 18.

Scheme 1b shows how 18 may be converted to a compound of Formula I of the present invention for an embodiment where n=0 in Formula I.

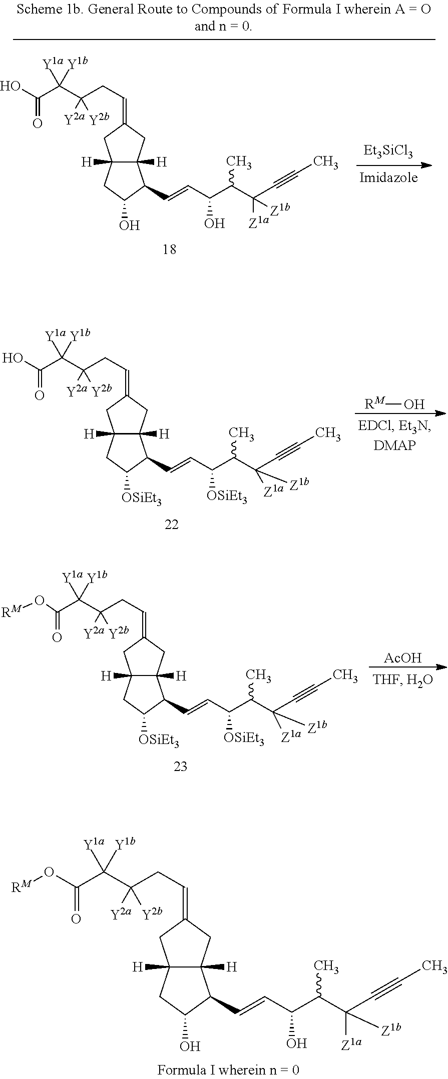

As outlined in Scheme 1b, 18 may be treated with Et₃SiCl to yield the protected di-ether 22 which upon esterification in the presence of the desired alcohol R^{M}OH affords ester 23. Deprotection of 23 in the presence of mild acid leads to the desired compounds of Formula I wherein n=0.

Scheme 1c shows how the protected di-ether 22 may be converted to a compound of Formula I of the present invention for an embodiment where A=O and n=1 in Formula I.

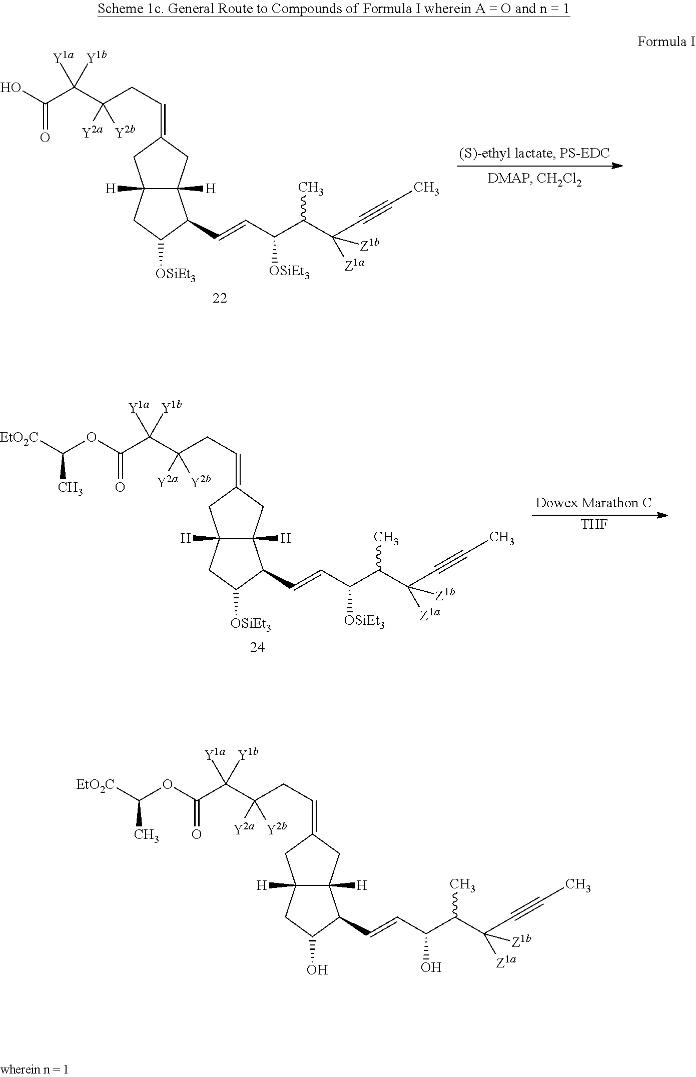

As shown in Scheme 1c above, esterification of disilyl ether 22 in the presence of (S)-ethyl lactate affords ester 24. Deprotection of 24 by the treatment with Dowex Marathon C resin affords compounds of Formula I wherein n=1.

Compounds of Formula A, wherein A=O and n=0 or 1, may be prepared according to Schemes 1a, 1b, 1c and 1d.

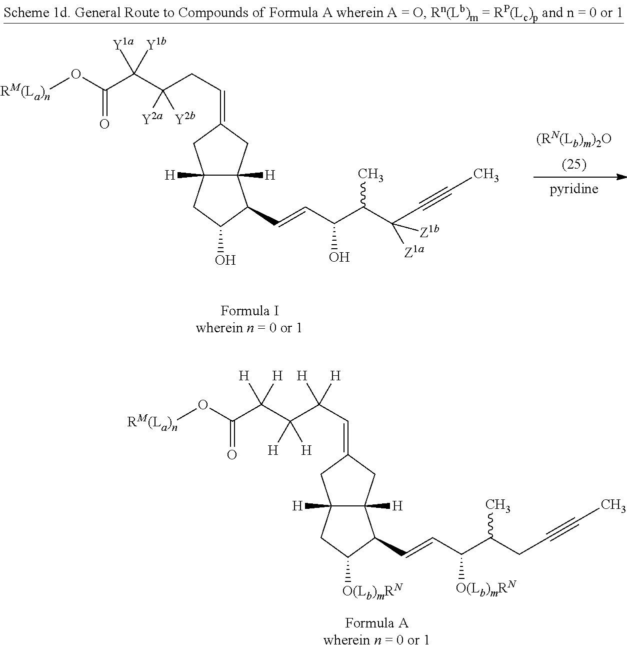

The preparation of a compound of Formula I, wherein n=0 or 1, is outlined in Schemes 1a, 1b and 1c above. Treatment of a compound of Formula I, wherein n=0 or 1, with the appropriate anhydride 25 in the presence of base such as pyridine leads to a compound of Formula A, wherein n=0 or 1.

Appropriately deuterated intermediate 12 may be prepared in a manner analogous to that described in Japanese Patent Application 2001309366. Intermediate 30 (Z^{1a/1b}=D) may be prepared as shown below in Scheme 2a and described by Schulte, K E et al., Chem. Ber. 1954, 87: 964-970. The preparation of intermediate 12 is depicted in the following Scheme 2b.

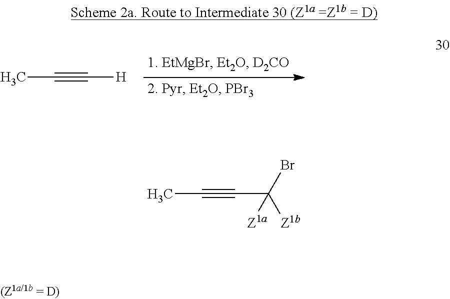

As shown in Scheme 2a above, coupling of prop-1-yne with d2-formaldehyde in the presence of ethyl magnesium bromide, followed by treatment with PBr₃ affords intermediate 30 wherein Z^{1a} and Z^{1b} are each deuterium.

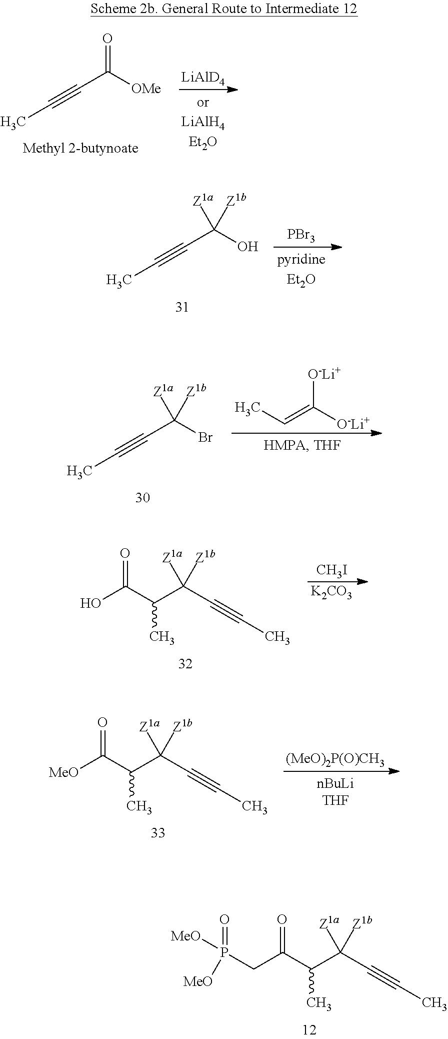

As depicted in Scheme 2 b, appropriately deuterated alcohol 31 may be prepared by treatment of methyl 2-butynoate with LiAlH₄ or LiAlD₄. Conversion of alcohol 31 to bromide 30 in the presence of PBr₃ followed by coupling with lithium prop-1-ene-1,1-bis(olate) affords carboxylic acid 32. Ester 33, resulting from treatment of 32 with methyl iodide, may be coupled with dimethyl methylphosphonate in the presence of n-butyl lithium to yield appropriately deuterated intermediates 12.

Compounds of Formula II may be synthesized according to Schemes 3a and 3b.

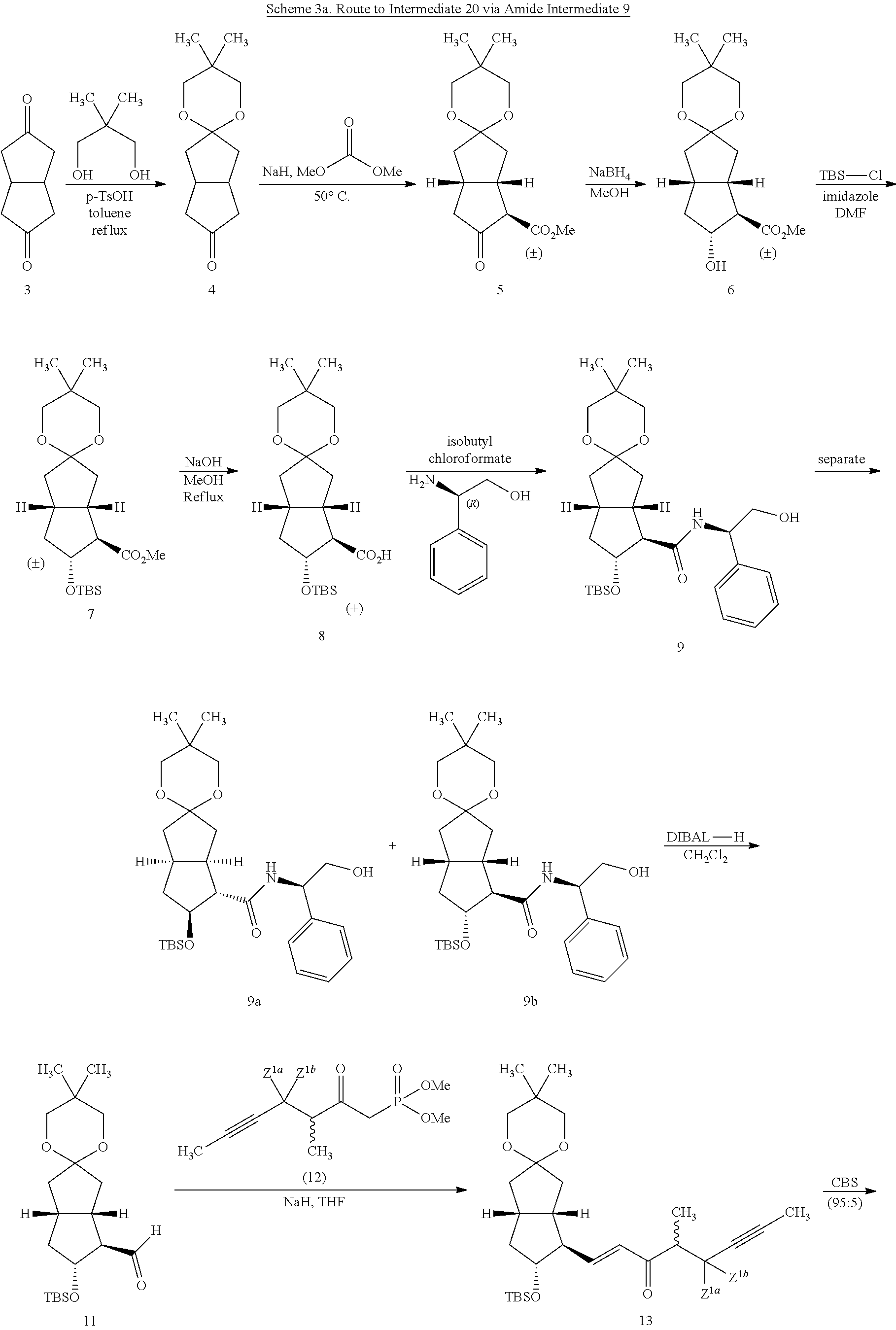

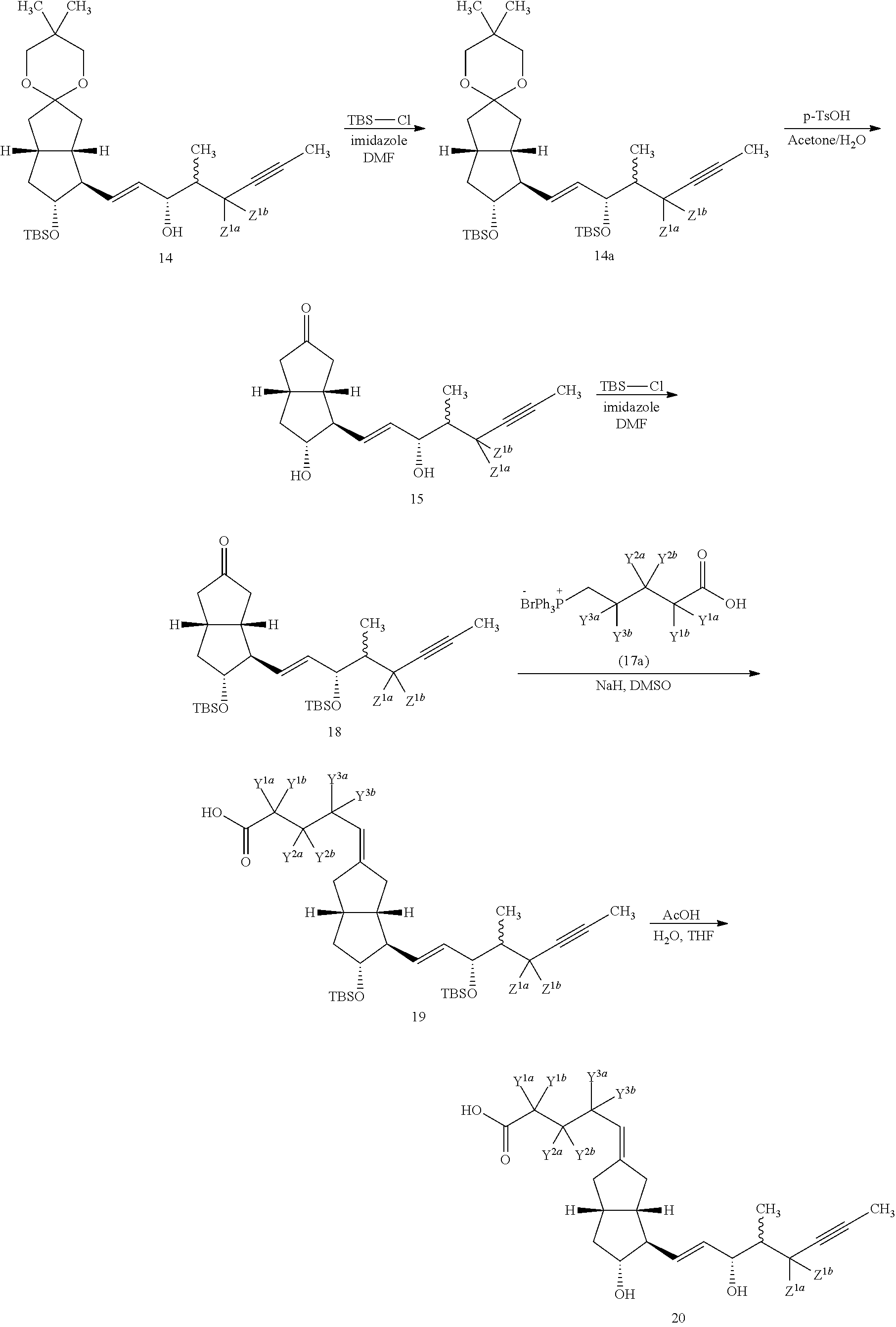

Scheme 3a above is based on chemistry that is known for the corresponding non-deuterated analogs and, in particular, on the process described in U.S. Pat. No. 4,925,956. which reports the preparation of optically active aldehyde 11 from the amide 9. According to the patent, the diastereomers corresponding to 9a and 9b are separated by column chromatography. The desired diastereomer 9b is then reduced with diisobutylaluminum hydride (DIBAL-H) to provide optically active 11. The Wittig reagent 17a wherein Y^{1a}=Y^{1b}=Y^{2a}=Y^{2b}=D (17b) can be prepared as shown in Example 6 below, and the Wittig reagent 17a wherein Y^{2a}=Y^{2b}=Y^{3a}=Y^{3b}=D (17c) can be prepared as shown in Example 9 below. Compounds may be prepared wherein the level of deuterium incorporation at each Y¹ and Y² position is greater than 90%. In a typical preparation, the level of deuterium incorporation at each Y¹ and Y² position is at least 95%, with each Y² position having at least 98% deuterium.

Scheme 3b shows how 20 may be converted to a compound of Formula II of the present invention for an embodiment where n=0 in Formula II.

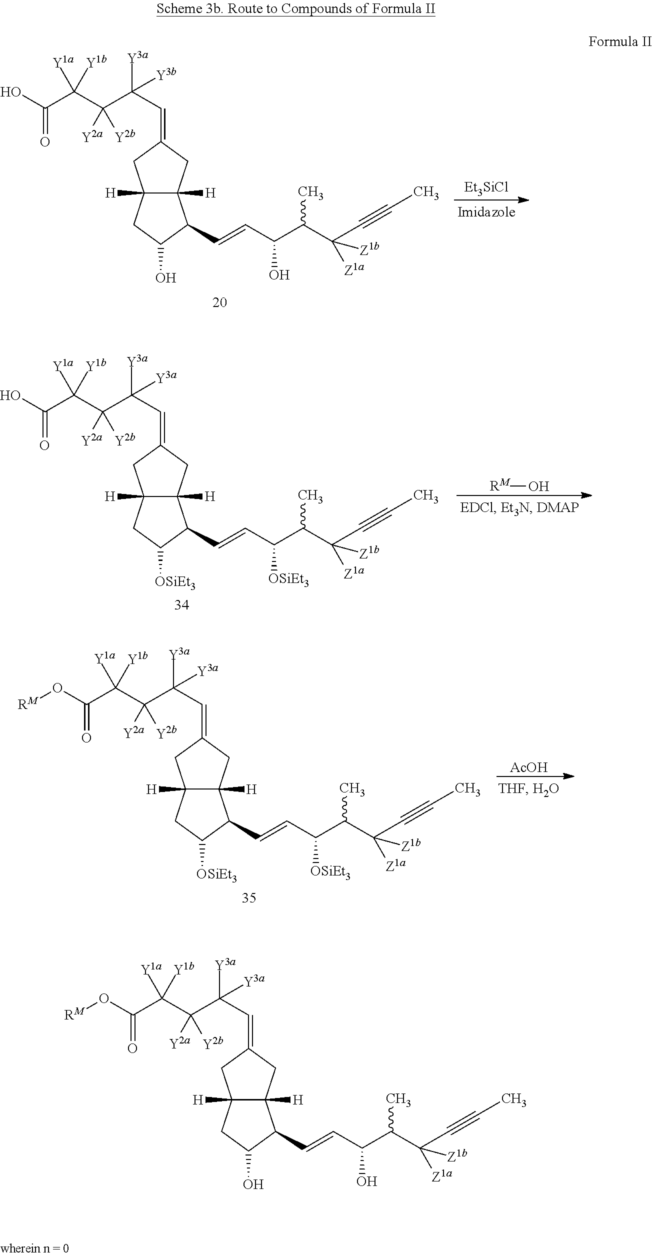

As outlined in Scheme 3b, 20 may be converted to compounds of Formula II wherein n=0 through protection of the alcohol moieties by treatment with triethyl silyl chloride to yield intermediate 34 followed by ester formation in the presence of the appropriate alcohol to afford intermediate 35 and subsequent deprotection of the ethers in the presence of mild acid.

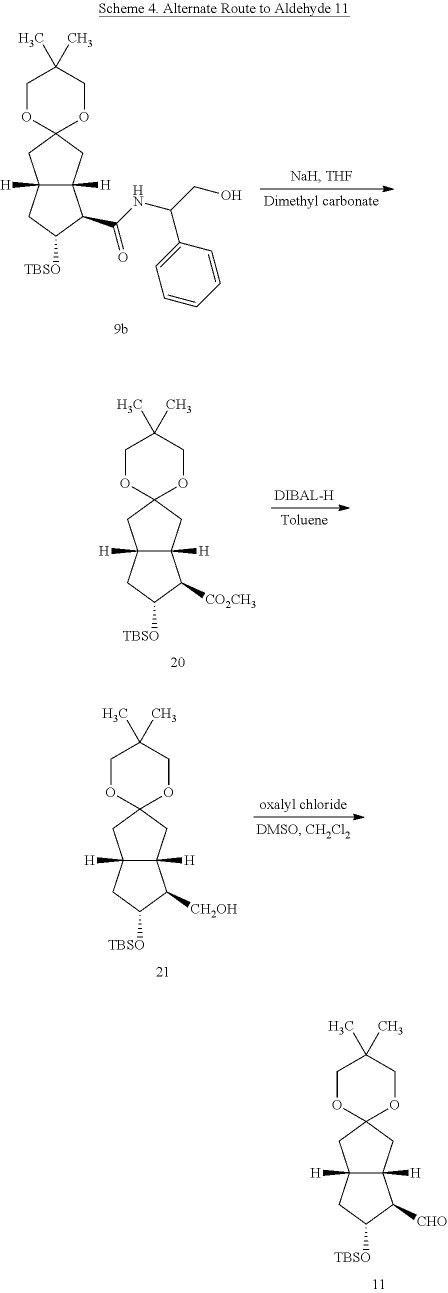

Scheme 4 above illustrates another approach to aldehyde 11. The amide 9b may be converted to the methyl ester 20, which then may be reduced with DIBAL-H to the alcohol 21. Treatment of 21 with oxalyl chloride and DMSO furnishes the aldehyde 11.

Other approaches to synthesizing compounds of the formulae herein (e.g., Formula I or II) can readily be adapted from references cited herein. Variations of these procedures and their optimization are within the skill of the ordinary practitioner.

The specific approaches and compounds shown above are not intended to be limiting. The chemical structures in the schemes herein depict variables that are hereby defined commensurately with chemical group definitions (moieties, atoms, etc.) of the corresponding position in the compound formulae herein, whether identified by the same variable name (i.e., Y^{1a}, Y^{1b}, Y^{2a}, Y^{2b}, Z^{1a}, or Z^{1b}) or not. The suitability of a chemical group in a compound structure for use in synthesis of another compound structure is within the knowledge of one of ordinary skill in the art.

Combinations of substituents and variables envisioned by this invention are only those that result in the formation of stable compounds.

The synthetic schemes and examples herein describe certain deuterated compounds that are useful as synthetic intermediates for making compounds of Formula I or Formula II. Thus the invention also provides such a compound that is selected from the following:

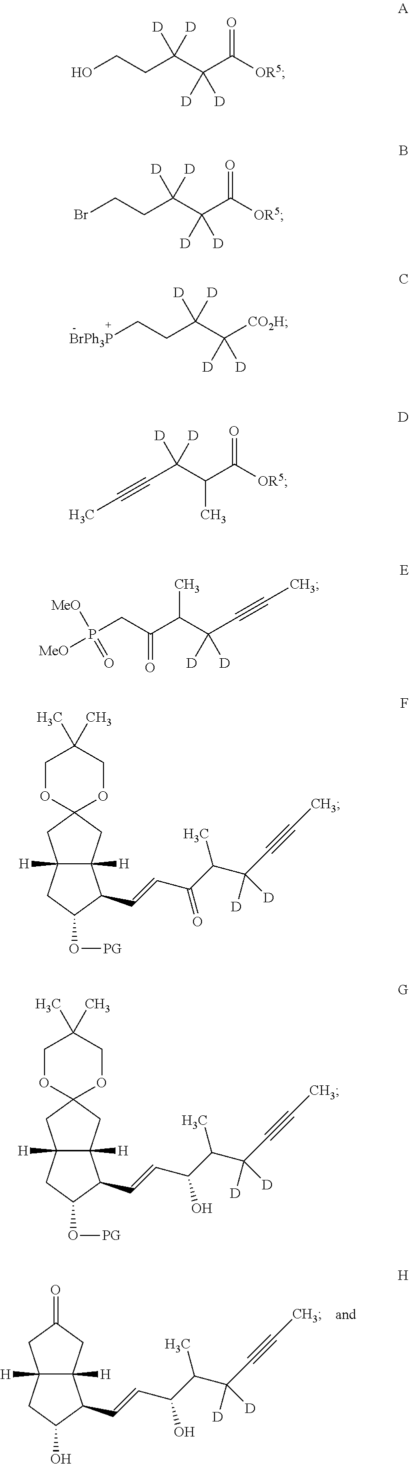

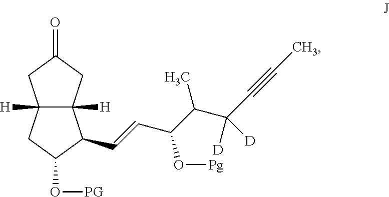

where R⁵ is hydrogen, deuterium, or a C₁-C₈ group and Pg is an alcohol protecting group. Examples of the C₁-C₈ group include C₁-C₆ alkyl such as methyl, ethyl, propyl and aralkyl such as benzyl. Examples of the Pg group include tert-butyldimethylsilyl (TBS), triisopropylsilyl (TIPS), triethylsilyl (TES), and tetrahydropyranyl (THP).

### Compositions

The invention also provides compositions comprising an effective amount of a compound of Formula I or II, or a pharmaceutically acceptable salt thereof; and an acceptable carrier. In one embodiment, the composition is pyrogen-free. In another embodiment the composition of this invention is formulated for pharmaceutical use (“a pharmaceutical composition”), wherein the carrier is a pharmaceutically acceptable carrier. The carrier(s) must be “acceptable” in the sense of being compatible with the other ingredients of the formulation and, in the case of a pharmaceutically acceptable carrier, not deleterious to the recipient thereof in amounts typically used in medicaments.

Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

If required, the solubility and bioavailability of the compounds of the present invention in pharmaceutical compositions may be enhanced by methods well-known in the art. One method includes the use of lipid excipients in the formulation. See “Oral Lipid-Based Formulations: Enhancing the Bioavailability of Poorly Water-Soluble Drugs (Drugs and the Pharmaceutical Sciences),” David J. Hauss, ed. Informa Healthcare, 2007; and “Role of Lipid Excipients in Modifying Oral and Parenteral Drug Delivery: Basic Principles and Biological Examples,” Kishor M. Wasan, ed. Wiley-Interscience, 2006.

Another known method of enhancing bioavailability is the use of an amorphous form of a compound of this invention optionally formulated with a poloxamer, such as LUTROL™ and PLURONIC™ (BASF Corporation), or block copolymers of ethylene oxide and propylene oxide. See U.S. Pat. No. 7,014,866; and United States patent publications 20060094744 and 20060079502.

The pharmaceutical compositions of the invention include those suitable for oral, rectal, nasal, topical (including buccal and sublingual), vaginal or parenteral (including subcutaneous, intramuscular, intravenous and intradermal) administration. In certain embodiments, the compound of the formulae herein is administered transdermally (e.g., using a transdermal patch or iontophoretic techniques). Other formulations may conveniently be presented in unit dosage form, e.g., tablets and sustained release capsules, and in liposomes, and may be prepared by any methods well known in the art of pharmacy. See, for example, Remington: The Science and Practice of Pharmacy, Lippincott Williams & Wilkins, Baltimore, Md. (20th ed. 2000).

Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers or both, and then if necessary shaping the product.

In certain preferred embodiments, the compound is administered orally. Compositions of the present invention suitable for oral administration may be presented as discrete units such as capsules, sachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion, or packed in liposomes and as a bolus, etc. Soft gelatin capsules can be useful for containing such suspensions, which may beneficially increase the rate of compound absorption.

In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are administered orally, the active ingredient is combined with emulsifying and suspending agents. If desired, certain sweetening and/or flavoring and/or coloring agents may be added.

Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

Such injection solutions may be in the form, for example, of a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of this invention may be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, e.g.: Rabinowitz J D and Zaffaroni A C, U.S. Pat. No. 6,803,031, assigned to Alexza Molecular Delivery Corporation.

Topical administration of the pharmaceutical compositions of this invention is especially useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this invention may also be topically applied to the lower intestinal tract by rectal suppository formulation or in a suitable enema formulation. Topically-transdermal patches and iontophoretic administration are also included in this invention.

Preferred dosage forms include inhalable microparticle formulations, such as those formulations of iloprost which are used with the I-neb™ AAD® System or the Prodose® AAD® System, as well as those described for iloprost in PCT patent publication WO2006014930; and oral formulations, such as those described in United States patent publication US20050101673.

Application of the subject therapeutics may be local, so as to be administered at the site of interest. Various techniques can be used for providing the subject compositions at the site of interest, such as injection, use of catheters, trocars, projectiles, pluronic gel, stents, sustained drug release polymers or other device which provides for internal access.

Thus, according to yet another embodiment, the compounds of this invention may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the invention provides a method of coating an implantable medical device comprising the step of contacting said device with the coating composition described above. It will be obvious to those skilled in the art that the coating of the device will occur prior to implantation into a mammal.

According to another embodiment, the invention provides a method of impregnating an implantable drug release device comprising the step of contacting said drug release device with a compound or composition of this invention. Implantable drug release devices include, but are not limited to, biodegradable polymer capsules or bullets, non-degradable, diffusible polymer capsules and biodegradable polymer wafers.

According to another embodiment, the invention provides an implantable medical device coated with a compound or a composition comprising a compound of this invention, such that said compound is therapeutically active.

According to another embodiment, the invention provides an implantable drug release device impregnated with or containing a compound or a composition comprising a compound of this invention, such that said compound is released from said device and is therapeutically active.

Where an organ or tissue is accessible because of removal from the subject, such organ or tissue may be bathed in a medium containing a composition of this invention, a composition of this invention may be painted onto the organ, or a composition of this invention may be applied in any other convenient way.

In another embodiment, a composition of the present invention further comprises a second therapeutic agent. The second therapeutic agent includes any compound or therapeutic agent known to have or that demonstrates advantageous properties when administered with vascular dilators of systemic or pulmonary arterial vascular beds or by platelet aggregation inhibitors. Such agents include those indicated as being useful in combination with iloprost which are described in detail in PCT patent publications WO1988001867; WO2005030187: WO2006014930: WO2005009446: WO2004019952; WO2000002450; WO1992013537; WO1997006806: and WO1998037894: and in United States Patent publications US20020128314; US20030139372; US20030162824; US20030216474; US20040033223; US20040052760; US 20040058940; US20040266880; US20050009847; US20050070596; US 20050080140; US20050101673; US20050106151; US20050119330; US20050239719; US20050239842; US20050239867; US20060183684; US20060160213.

In one embodiment, the second therapeutic agent is an agent useful in the treatment or prevention of a disease or condition selected from pulmonary arterial hypertension, Raynaud's phenomenon secondary to systemic sclerosis, contrast-mediated nephropathy, or lung cancer.

Even more specifically, the second therapeutic agent co-formulated with a compound of this invention is an agent useful in the treatment of pulmonary arterial hypertension.

In one embodiment, the second therapeutic agent is selected from a phosphodiesterase V inhibitor or an endothlin-1 antagonist. In another embodiment, the phosphodiesterase V inhibitor is sildenafil. In still another embodiment, the endothlin-1 antagonist is bosentan.

In another embodiment, the invention provides separate dosage forms of a compound of this invention and a second therapeutic agent that are associated with one another. The term “associated with one another” as used herein means that the separate dosage forms are packaged together or otherwise attached to one another such that it is readily apparent that the separate dosage forms are intended to be sold and administered together (within less than 24 hours of one another, consecutively or simultaneously).

In the pharmaceutical compositions of the invention, the compound of the present invention is present in an effective amount. As used herein, the term “effective amount” refers to an amount which, when administered in a proper dosing regimen, is sufficient to reduce or ameliorate the severity, duration or progression of the disorder being treated, prevent the advancement of the disorder being treated, cause the regression of the disorder being treated, or enhance or improve the prophylactic or therapeutic effect(s) of another therapy.

The interrelationship of dosages for animals and humans (based on milligrams per meter squared of body surface) is described in Freireich et al., (1966) Cancer Chemother Rep 50: 219. Body surface area may be approximately determined from height and weight of the subject. See, e.g., Scientific Tables, Geigy Pharmaceuticals, Ardsley, N.Y., 1970, 537. An effective unit dose amount of a compound of this invention can range from about 1 mg/kg body weight to about 500 mg/kg weight, more preferably 1 mg/kg to about 250 mg/kg, more preferably 1 mg/kg to about 75 mg/kg. Unit doses can be administered from once to nine times per day. Effective doses will also vary, as recognized by those skilled in the art, depending on the diseases treated, the severity of the disease, the route of administration, the sex, age and general health condition of the subject, excipient usage, the possibility of co-usage with other therapeutic treatments such as use of other agents and the judgment of the treating physician.

For pharmaceutical compositions that comprise a second therapeutic agent, an effective amount of the second therapeutic agent is between about 20% and 100% of the dosage normally utilized in a monotherapy regime using just that agent. Preferably, an effective amount is between about 70% and 100% of the normal monotherapeutic dose. The normal monotherapeutic dosages of these second therapeutic agents are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are entirely incorporated herein by reference.

It is expected that some of the second therapeutic agents referenced above will act synergistically with the compounds of this invention. When this occurs, its will allow the effective dosage of the second therapeutic agent and/or the compound of this invention to be reduced from that required in a monotherapy. This has the advantage of minimizing toxic side effects of either the second therapeutic agent of a compound of this invention, synergistic improvements in efficacy, improved ease of administration or use and/or reduced overall expense of compound preparation or formulation.

### Methods of Treatment

According to another embodiment, the invention provides a method of treating a subject suffering from or susceptible to a disease that is beneficially treated by iloprost comprising the step of administering to said subject an effective amount of a compound of Formula I or II or a pharmaceutically acceptable salt thereof, or a composition of this invention. Such conditions and diseases are well known in the art and include embolism-linked and other skin diseases, pulmonary hypertension, fibrosis-related diseases such as scleroderma, cerebral malaria, poor venous flow, bone diseases (such as bone marrow edema, osteonecrosis and osteoarthritis) syncytial virus infection, pruritic or atopic symptoms, inflammatory disorders, CNS disorders, as well as others disclosed in US 20050080140; US20030139372; US20030216474; US20040266880; US20050009847; US20050101673; WO1988001867; WO1992013537; WO2000002450; WO2004019952; and WO2006014930.

In a preferred embodiment, the method of this invention is used to treat a subject suffering from or susceptible to a disease or condition selected from pulmonary arterial hypertension, Raynaud's phenomenon secondary to systemic sclerosis, contrast-mediated nephropathy, or lung cancer. Methods delineated herein include those wherein the subject is identified as in need of a particular stated treatment. Identifying a subject in need of such treatment can be in the judgment of a subject or a health care professional and can be subjective (e.g. opinion) or objective (e.g. measurable by a test or diagnostic method).

In another embodiment, the invention provides a method of modulating the activity of a prostacyclin receptor in a cell comprising contacting the cell with one or more compounds of any of the formulae herein.

In another embodiment, the above method of treatment comprises the further step of co-administering to the subject one or more second therapeutic agents. The choice of second therapeutic agent may be made from any second therapeutic agent known to be useful for co-administration with iloprost. Such agents are specifically include any of those set forth above for use in pharmaceutical combinations of the invention.

In particular, the combination therapies of this invention include: treatment of erectile dysfunction in combination with a 15-hydroxyprostaglndindehydrogenase inhibitor; as an antithrombotic in combination with a betaine; treatment of angina, high blood pressure, pulmonary hypertension, congestive heart failure, chronic obstructive pulmonary disease (COPD), pulmonary heart disease, right ventricular failure, atherosclerosis, permeability conditions of reduced cardiovascular patency, peripheral vascular illnesses, cerebral apoplexy, bronchitis, allergic asthma, chronic asthma, allergic rhinitis, glaucoma, irritable bowel syndrome, tumors, kidney failure, cirrhosis of the liver and for treating male or female sexual problems each in combination with a phosphodiesterase V inhibitor; treating an inflammation related cardiovascular condition in combination with a COX-1 or COX-2 inhibitor; increasing or maintaining hair thickness in combination with a 15-hydroxyprostaglndindehydrogenase inhibitor; treating multiple sclerosis in combination with a cannabidiol derivative; treating a bacterial infection in combination with an α1-antitrypsin or serine protease inhibitor; treating a lung proliferative vascular disorder in combination with a HMG-CoA reductase inhibitor; treating pulmonary hypertension in combination with thalidomide or a phosphodiesterase IV inhibitor; treating hypertension, complications in diabetes and metabolic syndrome in combination with a blood pressure lowering agent; or treating pulmonary arterial hypertension in combination with an endothelin receptor antagonist, a phosphodiesterase inhibitor or a calcium channel blocker.

Other combination therapies useful in this invention are those combination therapies that employ iloprost and which are, described in US 20020128314; US 20040033223; US 20040058940; US20030162824; US20040052760; US20050070596; US20050106151; US20050119330; US20050239719; US20050239842; US20050239867; US20060160213; US20060183684; WO1997006806: WO1998037894: WO2005009446: and WO2005030187.

In a specific embodiment, the invention provides a method of treating a subject suffering from pulmonary arterial hypertension and comprises the step of co-administering to the subject a compound of Formula I or II or a pharmaceutically acceptable salt thereof, and a second therapeutic agent selected from a phosphodiesterase V inhibitor or an endothelin-1 antagonist. In a more specific embodiment, the phosphodiesterase V inhibitor is sildenafil. In another more specific embodiment, the endothelin-1 antagonist is bosentan.

The term “co-administered” as used herein means that the second therapeutic agent may be administered together with a compound of this invention as part of a single dosage form (such as a composition of this invention comprising a compound of the invention and an second therapeutic agent as described above) or as separate, multiple dosage forms. Alternatively, the additional agent may be administered prior to, consecutively with, or following the administration of a compound of this invention. In such combination therapy treatment, both the compounds of this invention and the second therapeutic agent(s) are administered by conventional methods. The administration of a composition of this invention comprising both a compound of the invention and a second therapeutic agent to a subject does not preclude the separate administration of that same therapeutic agent, any other second therapeutic agent or any compound of this invention to said subject at another time during a course of treatment.

Effective amounts of these second therapeutic agents are well known to those skilled in the art and guidance for dosing may be found in patents and published patent applications referenced herein, as well as in Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), and other medical texts. However, it is well within the skilled artisan's purview to determine the second therapeutic agent's optimal effective-amount range.

In one embodiment of the invention, where a second therapeutic agent is administered to a subject, the effective amount of the compound of this invention is less than its effective amount would be where the second therapeutic agent is not administered. In another embodiment, the effective amount of the second therapeutic agent is less than its effective amount would be where the compound of this invention is not administered. In this way, undesired side effects associated with high doses of either agent may be minimized. Other potential advantages (including without limitation improved dosing regimens and/or reduced drug cost) will be apparent to those of skill in the art.

In yet another aspect, the invention provides the use of a compound of Formula I or a pharmaceutically acceptable salt thereof, alone or together with one or more of the above-described second therapeutic agents, in the manufacture of a medicament, either as a single composition or as separate dosage forms, for treatment or prevention in a subject of a disease, disorder or symptom set forth above. Another aspect of the invention is a compound of the formulae herein for use in the treatment or prevention in a subject of a disease, disorder or symptom thereof delineated herein.

### EXAMPLES

### Example 1

### Synthesis of (3α′S,4′R,5′R,6α′R)-5′-(tert-butyldimethylsilyloxy)-5,5-dimethylhexahydro-1′H-spiro[[1,3]dioxane-2,2′-pentalene]-4′-carbaldehyde (11)

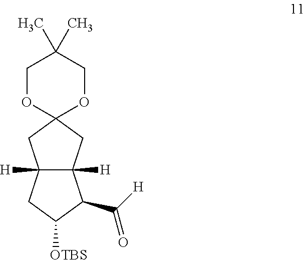

### Step 1. (2,2-dimethyltrimethylenedioxy)-cis-bicyclo[3.3.0]octan-3,7-dione (4)

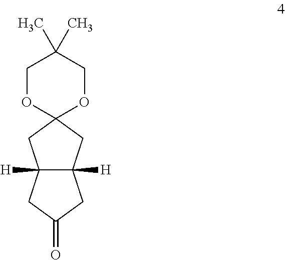

To the solution of the cis-Bicyclo[3.3.0]octan-3,7-dione, 3 (25 g, 181.06 mmol) in toluene (300 mL) was added 2,2-dimethyl-1,-propanediol (18.9 g, 181.06 mmol) and p-toluenesulfonic acid monohydrate (catalytic amount) and the solution was stirred at room temperature overnight. The reaction mixture was concentrated and the crude was subjected to column chromatography to give the monoprotected ketone, 4 (17.8 g, 44%). ¹HNMR (300 MHz, CDCl₃): δ 0.94 (s, 6H), 1.80 (dd, 2H), 2.13-2.70 (m, 6H), 2.80-2.90 (m, 2H), 3.52 (s, 2H), 3.65 (s, 2H). MS (M+H): 225.

### Step 2. (±) (2,2-Dimethyltrimethylenedioxy)-cis-bicyclo[3.3.0]octan-3-one-2-carboxylic acid methyl ester (5)

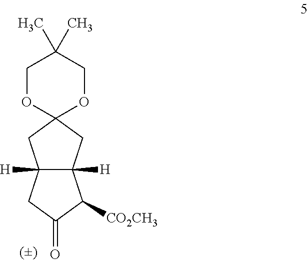

To a suspension of sodium hydride (2.33 g, 53.5 mmol) in dimethyl carbonate (80 mL) was added the mono-protected ketone, 4 (10 g, 44.6 mmol) dissolved in dimethyl carbonate (20 mL) and the solution was stirred at 50° C. overnight. The mixture was cooled, the excess sodium hydride was quenched with methanol and the mixture was neutralized with acetic acid. The product was extracted with dichloromethane (3×50 mL) and concentrated under vacuum to give the crude product. The crude was purified by column chromatography to give the product, 5. (7.0 g, 56%). ¹H NMR (300 MHz, CDCl₃): δ 0.93 (s, 6H), 1.50-1.90 (m, 3H), 2.04-2.10 (m, 1H), 2.20-2.60 (m, 4H), 3.30 (d, 1H), 3.44-3.59 (m, 4H), 3.77 (s, 3H), 10.35 (b s, 1H). MS (M+H): 283.

### Step 3. (±) 7,7-(2,2-Dimethyltrimethylenedioxy)-3-α-hydroxy-cis-bicyclo[3.3.0]octane-2-β-carboxylic acid methyl ester (6)

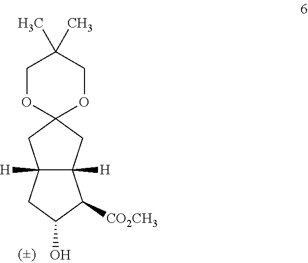

To the solution of the methyl ester, 5 (7 g, 24.8 mmol) in methanol (80 mL) at −40° C. was added NaBH₄ (1.87 g, 49.6 mmol) and the solution was stirred for 2 hours at the same temperature. Acetone (2 mL) was added to the reaction mixture and the solution was neutralized with satd. oxalic acid (5 mL). The solvents were evaporated and the residue was extracted with dichloromethane (2×25 mL). The organic layer was dried over sodium sulfate and evaporated to give the product, 6. (5 g, 71%). ¹H NMR (300 MHz, CDCl₃): δ 0.96 (s, 6H), 1.52-1.72 (m, 1H), 1.90-2.04 (m, 1H), 2.09-2.30 (m, 4H), 2.43-2.90 (m, 3H), 3.40-3.56 (m, 4H), 3.72 (s, 3H), 4.20-4.30 (m, 1H). MS (M+H): 285.

### Step 4. (±) 7,7-(2,2-Dimethyltrimethylenedioxy)-3-α-tert-butyldimethylsilyloxy-cis-bicyclo[3.3.0]octane-2-β-carboxylic acid methyl ester (7)

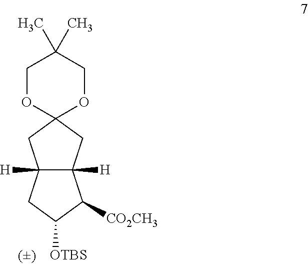

To a solution of the hydroxyl compound, 6 (3 g, 10.6 mmol) in DMF (40 mL) was added imidazole (1.72 g, 25.32 mmol) and tert-butyldimethylchlorosilane (1.91 g, 12.66 mmol) and the mixture was stirred at room temperature overnight. Water (10 mL) was added to the reaction mixture and the solution was extracted with ether (2×25 mL). The organic layer was dried over Na₂SO₄, concentrated and purified by column chromatography to give the product, 7. (3 g, 71%). ¹H NMR (300 MHz, CDCl₃) δ: 0.02 (s, 3H), 0.05 (s, 3H), 0.85 (s, 9H), 0.93 (s, 6H), 1.60-1.74 (m, 1H), 1.90-2.04 (m, 2H), 2.09-2.18 (m, 3H), 2.44-2.50 (m, 1H), 2.56-2.62 (m, 2H), 3.40-3.60 (m, 4H), 3.76 (s, 3H), 4.20-4.30 (m, 1H). MS (M+H): 399.

### Step 5. (±) 7,7-(2,2-Dimethyltrimethylenedioxy)-3-α-tert-butyldimethylsilyloxy-cis-bicyclo[3.3.0]octane-2-β-carboxylic acid (8)

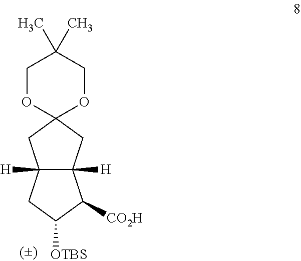

To the solution of the ester, 7 (3 g, 7.52 mmol) was added methanol (40 mL) and 5% NaOH (8.2 mL) and the solution was stirred at reflux for 1.5 hours. The solution was concentrated under vacuum, diluted with water (20 mL) and extracted with diethyl ether (25 mL). The mixture was cooled in an ice bath, acidified (pH=3) with 2N H₂SO₄ and extracted with diethyl ether (2×25 mL). The ether layer was dried over Na₂SO₄ and concentrated to give the acid, 8. (2.0 g, 69%). ¹HNMR (300 MHz, CDCl₃): δ 0.02 (s, 3H), 0.05 (s, 3H), 0.81 (s, 9H), 0.95 (s, 6H), 1.60-1.76 (m, 1H), 1.90-2.03 (m, 2H), 2.09-2.18 (m, 3H), 2.44-2.50 (m, 1H), 2.56-2.62 (m, 2H), 3.40-3.60 (m, 4H), 4.20-4.30 (m, 1H). MS (M+H): 385.

### Step 6. 7,7-(2,2-Dimethyltrimethylenedioxy)-3-α-tert-butyldimethylsilyloxy-cis-bicyclo[3.3.0]octane-2-β-carboxylic acid D-(−)-α-phenylglycinolamide (9b)

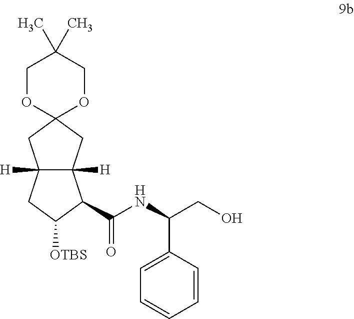

To the solution of the acid, 8 (4 g, 10.4 mmol) in acetone (30 mL), NEt₃ (1.3 mL, 9.32 mmol) was added and the solution was stirred for 5 minutes at 0° C. Isobutylchloroformate (1.2 mL, 8.73 mmol) dissolved in acetone (15 mL) was added to the reaction mixture and the solution was stirred for 20 minutes at the same temperature. D-(−)-α-phenylglycinol (1.17 g, 8.56 mmol) dissolved in acetone (15 mL) and acetonitrile (15 mL) was added dropwise to the reaction mixture and the solution is stirred for 24 hours at room temperature. The reaction mixture was concentrated, the residue was dissolved in dichloromethane (25 mL) and washed with brine (2×10 mL). The organic layer was dried over Na₂SO₄ and concentrated. The diastereomers were separated using column chromatography (Hexane: EtOAc=3:1) to give the required diastereomer, 9b. (1.82 g, 34%). ¹H NMR (300 MHz, CDCl₃): δ 0.06 (s, 6H), 0.83 (s, 9H), 0.95 (s, 6H), 1.42-1.90 (m, 1H), 1.90-2.03 (m, 2H), 2.09-2.18 (m, 3H), 2.44-2.50 (m, 1H), 2.56-2.62 (m, 2H), 3.40-3.60 (m, 4H), 3.88-3.96 (m, 2H), 4.14-4.23 (m, 1H), 5.05-5.17 (m, 1H), 6.50 (d, 1H), 7.26-7.34 (m, 5H). MS (M+H) m/z: 504. [α]_{D}=−27.39 (0.54 in CHCl₃).

### Step 7. Methyl (3a′S,4′R,5′R,6a′R)-5′-(tert-butyldimethylsilyloxy)-5,5-dimethyl hexahydro-1′H spiro[[1,3]dioxane-2,2′pentalene]-4′carboxylate (20)

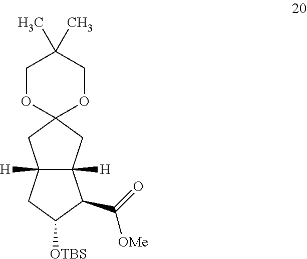

To a suspension of sodium hydride (60% in mineral oil, 2.33 g, 66.9 mmol) in tetrahydrofuran (60 mL) was added the amide 9b (10.0 g, 44.6 mmol)) dissolved in dimethyl carbonate (50 mL) at 0° C. and the solution was stirred at room temperature for 2 hours. The mixture was cooled, the excess sodium hydride was quenched with methanol and the mixture was neutralized with acetic acid. The product was extracted with methylene chloride (3×50 mL) and concentrated under vacuum to give the crude product. The crude was purified by column chromatography to give the product. (7.00 g, 56%). ¹H NMR (400 MHz, CDCl₃) δ: 0.02 (s, 3H), 0.08 (s, 3H), 0.85 (s, 9H), 0.99 (s, 3H), 1.0 (s, 3H), 1.50-1.56 (m, 1H), 1.80-1.95 (m, 2H), 2.09-2.28 (m, 3H), 2.45-2.50 (m, 1H), 2.60-2.70 (m, 2H), 3.40 (d, J=4.4 Hz, 2H), 3.50 (d, J=4.4 Hz, 2H), 3.68 (s, 3H), 4.24-4.30 (m, 1H). MS (M+H): 399.

### Step 8. (3a′S,4′R,5′R,6a′R)-5′-(tert-butyldimethylsilyloxy)-5,5-dimethyl hexahydro-1′H spiro[[1,3]]dioxane-2,2′pentalene]-4′-yl]methanol (21)

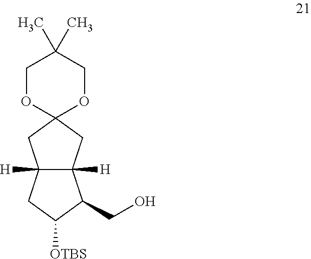

DIBAL-H 1.0 M in toluene (15.0 mmol, 15.0 mL) was added to a solution of methyl ester 20 from the previous step (3.00 g, 7.53 mmol) in methylene chloride (3 mL) at 78° C. and the solution was stirred for 2 hours at the same temperature. Methanol (2 mL) was added to the reaction mixture which was followed by the addition of aqueous potassium tartrate (1 mL). Then the mixture was filtered through Celite and was washed with methylene chloride (25 mL). The combined organic phases were dried over Na₂SO₄ and concentrated under vacuum to give the crude product. The crude was purified by column chromatography to give the product. (1.70 g, 70%). ¹H NMR (400 MHz, CDCl₃) δ: 0.05 (s, 3H), 0.07 (s, 3H), 0.87 (s, 9H), 0.93 (s, 3H), 0.98 (s, 3H), 1.40-1.58 (m, 1H), 1.73-2.01 (m, 3H), 2.01-2.20 (m, 3H), 2.20-2.42 (m, 1H), 3.42-3.49 (m, 4H), 3.64-3.67 (m, 2H), 3.82-3.90 (m, 1H). MS (M+H): 371.

### Step 9. (3a′S,4′R,5′R,6a′R)-5′-(tert-butyldimethylsilyloxy)-5,5-dimethyl hexahydro-1′H spiro[[1,3]]dioxane-2,2′pentalene]-4′carbaldehyde (11)

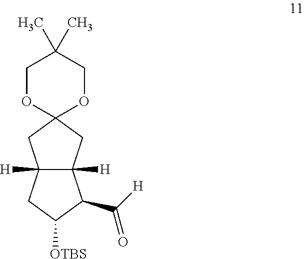

Oxalyl chloride (0.17 mL, 2.02 mmol) was dissolved in 5 mL of dichloromethane, cooled to 60° C. and mixed with dimethyl sulfoxide (0.21 mL, 4.05 mmol) in 2 mL of dichloromethane. After 10 minutes a solution of 0.50 g (1.35 mmol) of alcohol 21 from the previous step in dichloromethane (5 mL) was added and the reaction mixture was stirred at room temperature for 45 minutes. Triethylamine (0.58 mL, 4.18 mmol) in methylene chloride (2 mL) was added to the reaction mixture and the solution was warmed to room temperature. Water (20 mL) was added, and the organic phase was separated and washed with brine (2×10 mL). The combined organic phases were dried over Na₂SO₄ and concentrated under vacuum to give the crude product. (0.48 g, 96%). ¹H NMR (400 MHz, CDCl₃): δ 0.03 (s, 3H), 0.06 (s, 3H), 0.90 (s, 9H), 0.95 (s, 3H), 0.98 (s, 3H), 1.60-1.70 (m, 1H), 1.80-1.90 (m, 2H), 2.10-2.30 (m, 3H), 2.40-2.60 (m, 1H), 2.70-2.90 (m, 2H), 3.46-3.50 (m, 4H), 4.24-4.30 (m, 1H), 9.69 (d, J=2.4 Hz, 1H). MS (M+H) m/z: 369.

### Example 2

### Synthesis of 3-methyl-2-oxohept-5-ynylphosphonic acid dimethyl ester (12) (Z^{1a}=Z^{1b}=H)

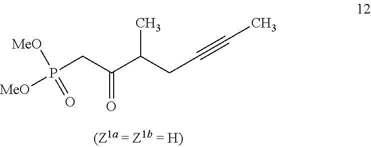

### Step 1. 2-methylhex-4-ynoic acid 32 (Z^{1a}=Z^{1b}=H)

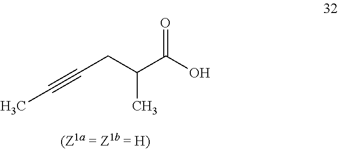

To a solution of diisopropylamine (32.75 mL, 233.09 mmol) in THF (100 mL) at −50° C. was added 1.6 M n-BuLi in hexane (94 mL) and the solution was stirred for 5 minutes. The reaction mixture was allowed to warm to −20° C. and the mixture was treated with a mixture of HMPA (15.7 mL) and propionic acid (6.75 mL, 90.23 mmol) dropwise. The reaction mixture was stirred at room temperature for 30 minutes. The contents were then cooled to 0° C. and 1-bromo-2-butyne, 30 (Z^{1a}=Z^{1b}=H) (10 g, 75.19 mmol) in THF (20 mL) was added to the reaction mixture and stirred at room temperature for 2 hours. The contents were poured into 10% HCl (20 mL) and the solution was extracted with ether (3×25 mL). The organic layer was dried over Na₂SO₄ and evaporated to give the product, 32 (Z^{1a}=Z^{1b}=H). (12 g). The crude was directly taken to next step. ¹H NMR (300 MHz, CDCl₃): δ 1.15 (d, 3H), 1.77 (t, 3H), 2.35 (m, 2H), 2.66 (m, 1H)

### Step 2. Methyl 2-methylhex-4-ynoate 33 (Z^{1a}=Z^{1b}=H)

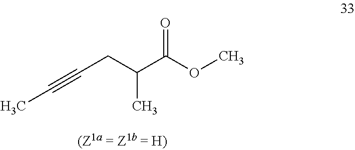

To the solution of the crude acid, 32 (Z^{1a}=Z^{1b}=H) (12 g, 95.1 mmol) in acetone (100 mL) was added MeI (8.9 mL, 142.68 mmol) and K₂CO₃ (26.3 g, 190.24 mmol) and the solution was stirred at room temperature overnight. The reaction mixture was evaporated and the contents were dissolved in water (25 mL). The solution was extracted with ether (3×25 mL), and the organic layer was dried over Na₂SO₄ and evaporated. The crude product was vacuum distilled to give pure product, 33 (Z^{1a}=Z^{1b}=H). (4.4 g, 33%). ¹H NMR (300 MHz, CDCl₃): δ 1.25 (d, 3H), 1.77 (t, 3H), 2.34 (m, 2H), 2.66 (m, 1H), 3.69 (s, 3H).

### Step 3. 3-methyl-2-oxohept-5-ynylphosphonic acid dimethyl ester, 12 (Z^{1a}=Z^{1b}=H)

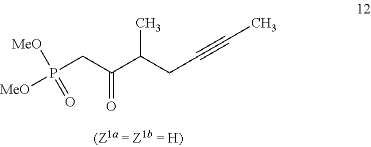

To a solution of dimethyl methylphosphonate (4.5 mL, 42.80 mmol) in THF (20 mL) was added 1.6 M n-BuLi in hexane (24 mL) dropwise and the solution was stirred at −78° C. for 30 minutes. The ester, 33 (Z^{1a}=Z^{1b}=H) (3.0 g, 21.40 mmol) dissolved in THF (10 mL) was added to the reaction mixture dropwise and the mixture was stirred at −78° C. for 3 hours and at ambient temperature for 1 hour. The reaction mixture was quenched with acetic acid (1 mL) added with saturated brine (30 mL), and was extracted with ether (3×10 mL). The ether layer was dried over Na₂SO₄ and evaporated to give the crude product. The crude product was vacuum distilled to give the pure product, 12 (Z^{1a}=Z^{1b}=H). (2 g, 40%). ¹H NMR (300 MHz, CDCl₃): δ 1.18 (d, 3H), 1.76 (t, 3H), 2.36 (m, 2H), 2.64 (m, 1H), 3.26 (d, 2H), 3.77 (s, 3H), 3.81 (s, 3H).

### Example 3

### Synthesis of 4,4-d₂-3-methyl-2-oxohept-5-ynylphosphonic acid dimethyl ester (12) (Z^{1a}=Z^{1b}=D)

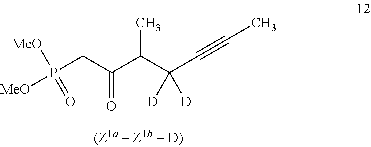

### Step 1. 1,1-d₂-But-2-yn-1-ol 31 (Z^{1a}=Z^{1b}=D)

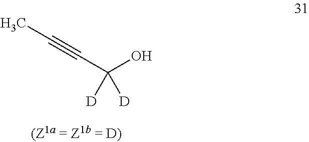

To a suspension of lithium aluminum deuteride (1.28 g, 30.57 mmol) in ether (60 mL) was added dropwise methyl 2-butynoate (5 g, 51 mmol) in ether (20 mL) at 0° C. The reaction mixture was stirred was stirred for 1 hour at room temperature and quenched with satd. ammonium chloride (1 mL). The ether layer was filtered, dried over Na₂SO₄ and concentrated. The residue was vacuum distilled to give the alcohol, 31 (Z^{1a}=Z^{1b}=D). (2 g, 55%). ¹H NMR (300 MHz, CDCl₃): 1.85 (s, 3H)

### Step 2. 1,1-d₂-1-Bromo-but-2-yne 30 (Z^{1a}=Z^{1b}=D)

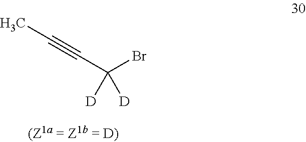

To a stirred solution of 1,1-d₂-but-2-yn-1-ol, 31 (Z^{1a}=Z^{1b}=D) (1.2 g, 16.64 mmol) in ether (10 mL) at 0° C. was added pyridine (4 mL, 49.92 mmol), and phosphorous tribromide (0.89 mL, 11.15 mmol) dropwise and the solution was warmed to reflux for 2 hours. The reaction mixture was cooled to 0° C., the contents were treated with satd. NaBr solution (10 mL) and extracted with ether (2×10 mL). The ether layer was dried over Na₂SO₄ and concentrated to give the product, 30 (Z^{1a}=Z^{1b}=D). (0.6 g, 30%). ¹H NMR (400 MHz): 1.88 (s, 3H)

### Step 3. 3,3-d₂-2-Methylhex-4-ynoic acid 32 (Z^{1a}=Z^{1b}=D)

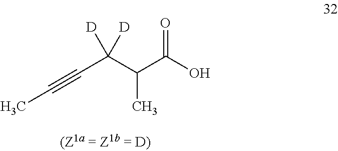

To a solution of diisopropylamine (1.93 mL, 13.77 mmol) in THF (10 mL) at −50° C. was added 1.2M n-BuLi in hexane (7.4 mL) and the solution was stirred for 5 minutes. The reaction mixture was allowed to warm to −20° C. and the mixture was treated with a mixture of HMPA (0.77 mL) and propionic acid (0.39 mL, 5.32 mmol) dropwise. The reaction mixture was stirred at room temperature for 30 minutes. The contents were then cooled to 0° C. and 1,1-d2-1-bromo-but-2-yne, 30 (Z¹a=Z^{1b}=D) (0.60 g, 4.44 mmol) was added to the reaction mixture and stirred at room temperature for 2 hours. The contents were poured into 10% HCl (5 mL) and the solution was extracted with ether (2×10 mL). The organic layer was dried over Na₂SO₄ and evaporated to give the product, 32 (Z^{1a}=Z^{1b}=D) (1.0 g). The crude was directly taken to next step. ¹H NMR (300 MHz, CDCl₃): δ 1.15 (d, 3H), 1.83 (s, 3H), 2.64 (q, 1H)

### Step 4. Methyl 3,3-d₂-Methylhex-4-ynoate 33 (Z^{1a}=Z^{1b}=D)

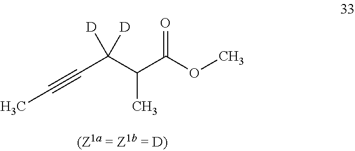

To the solution of the crude acid, 32 (Z^{1a}=Z^{1b}=D) (1.0 g, 11.53 mmol) in acetone (15 mL) was added MeI (1.07 mL, 17.30 mmol) and K₂CO₃ (3.18 g, 23.06 mmol) and the solution was stirred at room temperature overnight. The reaction mixture was evaporated and the contents were dissolved in water (10 mL). The solution was extracted with ether (2×10 mL) and the organic layer was dried over Na₂SO₄ and evaporated. The crude was purified by vacuum distillation to give the crude product, 33 (Z^{1a}=Z^{1b}=D) (0.6 g). The crude was directly taken to next step. ¹H NMR (300 MHz, CDCl₃): δ 1.15 (d, 3H), 1.84 (s, 3H), 2.65 (q, 1H), 3.69 (s, 3H)

### Step 5. 4,4-d₂-3-Methyl-2-oxohept-5-ynylphosphonic acid dimethyl ester 12 (Z^{1a}=Z^{1b}=D)

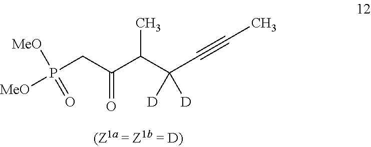

To a solution of dimethyl methylphosphonate (0.66 mL, 8.56 mmol) in THF (10 mL) was added 1.2 M n-BuLi in hexane (6.42 mL) dropwise and the solution was stirred at −78° C. for 30 minutes. The ester, 33 (Z^{1a}=Z^{1b}=D) (0.60 g, 4.28 mmol) dissolved in THF (5 mL) was added to the reaction mixture dropwise and the mixture was stirred at −78° C. for 3 hours and at ambient temperature overnight. The reaction mixture was quenched with acetic acid (0.5 mL) added with saturated brine (10 mL), and was extracted with ether (2×5 mL). The ether layer was dried over Na₂SO₄ and evaporated to give to give the crude product, 12 (Z^{1a}=Z^{1b}=D) (0.40 g).

### Example 4

### Synthesis of (3aS,4R,5R,6aR)-5-(tert-butyldimethylsilyloxy)-4-((3S,E)-3-(tert-butyldimethylsilyloxy)-4-methyloct-1-en-6-ynyl)hexahydropentalen-2(1H)-one (18)

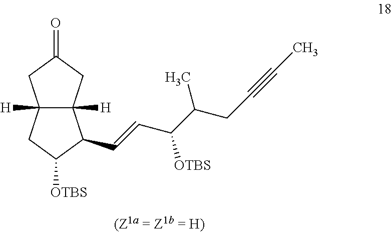

### Step 1. [(3a′S,4′R,5′R,6a′R)-5′-(tert-Butyldimethylsilyloxy)-5,5-dimethyl hexahydro-1′H spiro[[1,3]]dioxane-2,2′pentalene]-4′-yl]-4-methyloct-1-en-6-yn-3-one (13) (Z^{1a}=Z^{1b}=H)

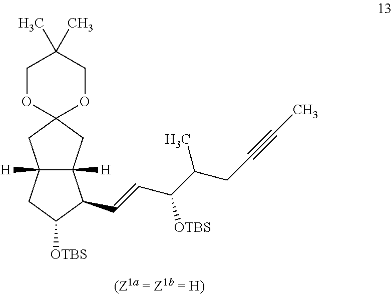

To a suspension of 55% sodium hydride (0.108 g, 2.71 mmol) in tetrahydrofuran (12 mL) was added dimethyl 3-methyl-2-oxohept-5-ynylphosphonate (Compound 12 where Z^{1a}=Z^{1b}=H) (0.630 g, 2.71 mmol) in tetrahydrofuran (8 mL). The solution was stirred for 30 minutes at room temperature and then a solution of aldehyde 11 from Example 1 (1.00 g, 2.71 mmol) in tetrahydrofuran (8 mL) was added. After 2 hours the reaction mixture was neutralized with acetic acid (0.20 mL) and concentrated under vacuum. The residue was taken up in methylene chloride (20 mL) and washed with brine solution (2×20 mL). The organic layer was dried over Na₂SO₄ and concentrated under vacuum to give the crude product. The crude was purified by column chromatography to give the titled product (0.90 g, 70%). ¹H NMR (400 MHz, CDCl₃): δ −0.03 (s, 3H), 0.01 (s, 3H), 0.85 (s, 9H), 0.95 (s, 3H), 0.98 (s, 3H), 1.27 (d, J=7.2 Hz, 3H), 1.40-1.66 (m, 1H), 1.70 (s, 3H), 1.70-1.90 (m, 2H), 2.10-2.50 (m, 8H), 2.80-2.90 (m, 1H), 3.46 (s, 2H), 3.49 (s, 2H), 3.80-3.90 (m, 1H), 6.20 (dd, J=3.2 Hz, 0.8 Hz, 1H), 6.70 (dd, J=15.6 Hz, 8.2 Hz, 1H).

### Step 2. (E)-[(3a′S,4′R,5′R,6a′R)-5′-(tert-butyldimethylsilyloxy)-5,5-dimethyl hexahydro-1′H spiro[[1,3]]dioxane-2,2′pentalene]-4′-yl]-4-methyloct-1-en-6-yn-3-ol (14) (Z^{1a}=Z^{1b}=H)

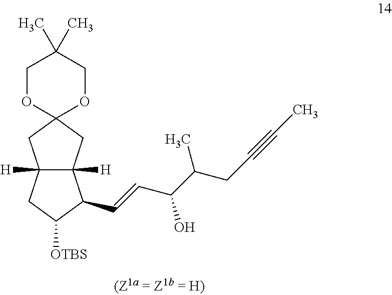

Compound 13 (Z^{1a}=Z^{1b}=H) (2.00 g, 4.22 mmol) was dissolved in methanol (58 mL) and cooled to −78° C. Cerium (III) chloride heptahydrate (1.58 g, 4.22 mmol) was added and the reaction mixture was stirred at approximately 78° C. for 1 hour. Sodium borohydride (0.291 g, 7.59 mmol) was added to the reaction mixture at the same temperature and stirred for another 45 minutes at approximately 78° C. After addition of acetone (2 mL), the reaction mixture was slowly warmed to room temperature, neutralized with acetic acid (0.2 mL) and the solvent was evaporated under vacuum. The residue was dissolved in dichloromethane and washed with water. The organic layer was dried with Na₂SO₄ and concentrated under vacuum to give the crude product. The crude was purified by column chromatography to give the titled product. (2.00 g, 95%). ¹H NMR (400 MHz, CDCl₃): δ −0.03 (s, 3H), −0.01 (s, 3H), 0.80 (s, 9H), 0.90 (s, 3H), 1.00 (s, 3H), 1.30 (d, J=7.2 Hz, 3H), 1.30-1.60 (m, 4H), 1.60-1.80 (m, 2H), 1.80 (s, 3H), 1.90-2.30 (m, 6H), 2.30-2.50 (m, 1H), 3.45 (s, 2H), 3.50 (s, 2H), 3.70-3.80 (m, 1H), 3.90-4.00 (m, 0.45H), 4.10-4.20 (m, 0.55H), 5.40-5.60 (m, 2H)

### Step 3. (3aS,4R,5R,6aR)-5-Hydroxy-4-((3S,E)-3-hydroxy-4-methyloct-1-en-6-ynyl)hexahydropentalen-2(1H)-one (15) (Z^{1a}=Z^{1b}=H)

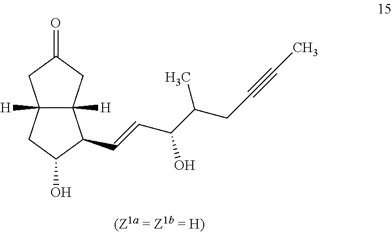

To a solution of acetal 14 (Z^{1a}=Z^{1b}=H) (0.50 g, 1.05 mmol) in acetone (4 mL) and H₂O (1.5 mL) was added p-toluenesulfonic acid (10 mg). The mixture was stirred at ambient temperature for 12 hours. Then aqueous NaHCO₃ (5 mL) was added and the aqueous phase was extracted with diethylether (2×10 mL). The combined organic phases were dried Na₂SO₄ and concentrated in vacuum to give crude product (0.500 g, 100%). ¹H NMR (400 MHz, CDCl₃): δ 1.30 (d, J=7.0 Hz, 3H), 1.40-1.80 (m, 2H), 1.80 (s, 3H), 2.08-2.60 (m, 7H), 2.60-2.70 (m, 1H), 2.70-2.90 (m, 1H), 3.90-4.20 (m, 2H), 5.40-5.60 (m, 2H).

### Step 4. (3aS,4R,5R,6aR)-5-(tert-Butyldimethylsilyloxy)-4-((3S,E)-3-(tert-butyldimethylsilyloxy)-4-methyloct-1-en-6-ynyl)hexahydropentalen-2(1H)-one (18) (Z^{1a}=Z^{1b}=H)

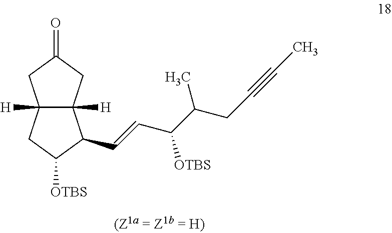

To a solution of 15 (Z^{1a}=Z^{1b}=H) (0.50 g, 1.80 mmol) in DMF (3 mL), imidazole (0.705 g, 10.8 mmol) was added. The mixture was stirred for 5 minutes at ambient temperature. Then tert-butyldimethylsilylchloride (0.660 g, 4.32 mmol) was added. The mixture was stirred at ambient temperature for 14 hours. Then aqueous NaHCO₃ (10 mL) was added and the aqueous phase was extracted with Et₂O (3×10 mL). The combined organic phases were dried Na₂SO₄ and concentrated in vacuum to give the crude product. The crude was purified by column chromatography to give the product (0.310 g, 55%). ¹H NMR (400 MHz, CDCl₃): δ −0.03 (s, 3H), −0.01 (s, 3H), 0.02 (s, 3H), 0.04 (s, 3H), 0.85 (s, 9H), 0.90 (s, 9H), 1.30 (s, 3H), 1.50-1.60 (m, 1H), 1.60-1.75 (m, 1H), 1.80 (s, 3H), 1.90-2.60 (m, 9H), 2.70-2.90 (m, 1H), 3.90-4.10 (m, 1.5H), 4.10-4.20 (m, 0.5H), 5.50-5.60 (m, 2H).

### Example 5

### Synthesis of (3aS,4R,5R,6aR)-5-(tert-Butyldimethylsilyloxy)-4-((3S,E)-3-(tert-butyldimethylsilyloxy)-5,5-d₂-4-methyloct-1-en-6-ynyl)hexahydropentalen-2(1H)-one (18) (Z^{1a}=Z^{1b}=D)

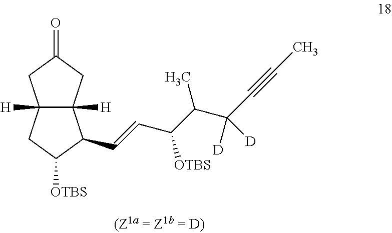

### Step 1. [(3a′S,4′R,5′R,6a′R)-5′-(tent-Butyldimethylsilyloxy)-5,5-dimethyl hexahydro-1′H Spiro[[1,3]]dioxane-2,2′pentalene]-4′-yl]-5,5-d₂-4-methyloct-1-en-6-yn-3-one (13) (Z^{1a}=Z^{1b}=D)

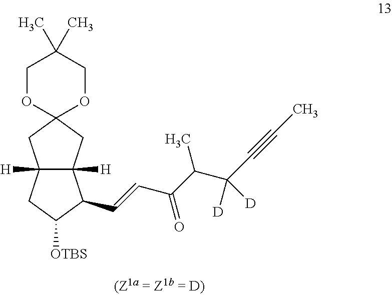

To a suspension of 55% sodium hydride (0.05 g, 1.30 mmol) in tetrahydrofuran (6 mL) was added 4,4-d2-3-methyl-2-oxohept-5-ynylphosphonic acid dimethyl ester (0.30 g, 1.30 mmol) in tetrahydrofuran (8 mL). The solution was stirred for 30 minutes at room temperature and then (3a′S,4′R,5′R,6a′R)-5′-(tert-butyldimethylsilyloxy)-5,5-dimethyl hexahydro-1′H spiro[[1,3]]dioxane-2,2′pentalene]-4′carbaldehyde (11) (0.48 g, 1.30 mmol) dissolved in tetrahydrofuran (4 mL) was added. After 2 hours the reaction mixture was neutralized with acetic acid (0.10 mL) and concentrated under vacuum. The residue was taken up in methylene chloride (10 mL) and washed with brine (2×10 mL). The organic layer was dried over Na₂SO₄ and concentrated under vacuum to give the crude product. The crude was purified by column chromatography to give the product (0.80 g, 64%). ¹H NMR (400 MHz, CDCl₃): δ −0.01 (s, 3H), −0.03 (s, 3H), 0.85 (s, 9H), 0.95 (s, 3H), 0.99 (s, 3H), 1.27 (s, 3H), 1.44-1.54 (m, 1H), 1.77 (d, J=1.60 Hz, 3H), 1.79-1.83 (m, 2H), 2.10-2.22 (m, 3H), 2.25-2.34 (m, 1H), 2.38-2.52 (m, 2H), 2.87-2.91 (m, 1H), 3.47 (s, 2H), 3.49 (m, 1H), 3.81-3.89 (m, 1H), 6.20 (ddd, J=15.6 Hz, 2.8 Hz, 0.4 Hz, 1H), 6.70 (dd, J=15.6 Hz, 8.40 Hz, 1H).

### Step 2. (E)-[(3a′S,4′R,5′R,6a′R)-5′-(tert-Butyldimethylsilyloxy)-5,5-dimethyl hexahydro-1′H Spiro[[1,3]]dioxane-2,2′pentalene]-4′-yl]-5,5-d₂-4-methyloct-1-en-6-yn-3-ol (14) (Z^{1a}=Z^{1b}=D)

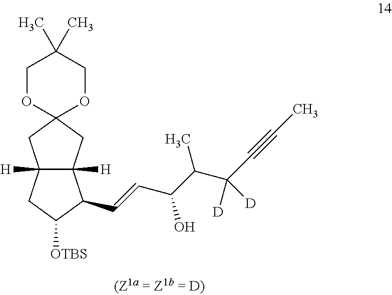

Compound 13 (Z^{1a}=Z^{1b}=D) (0.800 g, 1.68 mmol) was dissolved in methanol (23 mL) and cooled to approximately 78° C. Cerium (III) chloride heptahydrate (0.63 g, 1.68 mmol) was added and the reaction mixture was stirred at 78° C. for 1 hour. Sodium borohydride (0.116 g, 3.02 mmol) was added to the reaction mixture at the same temperature and stirred for another 45 minutes at 78° C. After addition of acetone (1 mL), the reaction mixture was slowly warmed to room temperature, neutralized with acetic acid (0.1 mL) and the solvent was evaporated under vacuum. The residue was dissolved in methylene chloride and washed with water. The organic layer was dried with Na₂SO₄ and concentrated under vacuum to give the crude product. The crude was purified by column chromatography to give the product. (0.60 g, 72%). ¹H NMR (400 MHz, CDCl₃): δ −0.03 (s, 3H), −0.01 (s, 3H), 0.90 (s, 9H), 0.95 (s, 3H), 1.00 (s, 3H), 1.50 (m, 1H), 1.60-1.80 (m, 3H), 1.80 (s, 3H), 2.10-2.30 (m, 6H), 2.30-2.40 (m, 1H), 3.50 (s, 4H), 3.70-3.80 (m, 1H), 3.90-4.00 (m, 1H), 4.00-4.10 (m, 1H), 5.40-5.70 (m, 2H).

### Step 3. (3aS,4R,5R,6aR)-4-((3R,E)-5,5-d₂-3-Hydroxy-4-methyloct-1-en-6-ynyl)-5-hydroxyhexahydropentalen-2(1H)-one (15) (Z^{1a}=Z^{1b}=D)

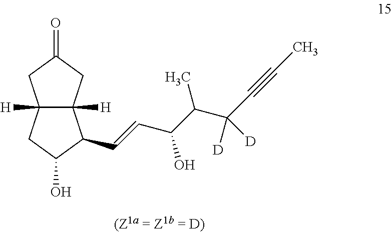

To a solution of Compound 14 (Z^{1a}=Z^{1b}=D) (1.80 g, 3.76 mmol) in acetone (14 mL) and H₂O (6 mL) was added p-toluenesulfonic acid (14 mg). The mixture was stirred at ambient temperature for 12 hours. Then aqueous NaHCO₃ (10 mL) was added and the aqueous phase was extracted with diethyl ether (2×10 mL). The combined organic phases were dried over Na₂SO₄ and concentrated under vacuum to give crude product (1.00 g, 100%). ¹H NMR (400 MHz, CDCl₃): δ 1.30 (d, J=7.0 Hz, 3H, 1.40-1.80 (m, 2H), 1.80 (s, 3H), 2.08-2.60 (m, 7H), 2.60-2.70 (m, 1H), 2.70-2.90 (m, 1H), 3.90-4.00 (m, 1.30H), 4.10-4.20 (m, 0.7H), 5.40-5.60 (m, 2H).

### Step 4. (3aS,4R,5R,6aR)-5-(tert-Butyldimethylsilyloxy)-4-((3S,E)-3-(tert-butyldimethylsilyloxy)-5,5-d₂-4-methyloct-1-en-6-ynyl)hexahydropentalen-2(1H)-one (18) (Z^{1a}=Z^{1b}=D)

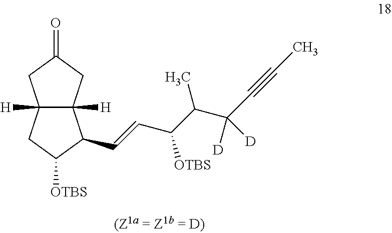

To a solution of Compound 15 (Z^{1a}=Z^{1b}=D) (1.00 g, 3.59 mmol) in DMF (6 mL), imidazole (1.41 g, 21.5 mmol) was added. The mixture was stirred for 5 minutes at ambient temperature. Then tert-butyldimethylsilylchloride (1.32 g, 8.97 mmol) was added. The mixture was stirred at ambient temperature for 14 hours. Then aqueous NaHCO₃ (10 mL) was added and the aqueous phase was extracted with diethyl ether (3×10 mL). The combined organic phases were dried over Na₂SO₄ and concentrated under vacuum to give the crude product. The crude was purified by column chromatography to give the product. (1.30 g, 72%). ¹H NMR (400 MHz, CDCl₃): δ −0.03 (s, 3H), −0.01 (s, 3H), 0.02 (s, 3H), 0.04 (s, 3H), 0.85 (s, 9H), 0.90 (s, 9H), 1.30 (s, 3H), 1.50-1.60 (m, 1H), 1.70-1.75 (m, 1H), 1.80 (s, 3H), 1.90-2.60 (m, 9H), 2.70-2.90 (m, 1H), 3.90-4.20 (m, 2H), 5.50-5.60 (m, 2H).

### Example 6

### Synthesis of (4-Carboxy-3,3,4,4-d₄-butyl)triphenylphosphonium bromide (17b) (Y^{1a/1b}=Y^{2a/2b}=D; Y^{3a/3b}=H)

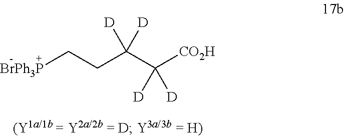

### Step 1. Ethyl 2,2,3,3-d₄-5-Hydroxypentanoate

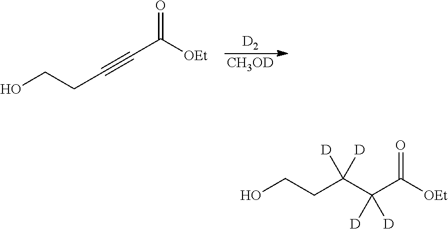

A solution of ethyl 5-hydroxypent-2-ynoate (16.0 g, 106.6 mmol, prepared according to the procedure described in J. Chem. Soc. Perkin Trans. I 1999, 2852-2863) in CH₃OD (80 mL) was subjected to deuterogenation conditions using deuterium gas (Isotec) at room temperature overnight. The reaction mixture was monitored by GC-MS. The reaction mixture was filtered through a Celite pad and the solution was concentrated to give the crude deuterated product (14.0 g, 82%) which was directly taken to next step. ¹H NMR (400 MHz, CDCl₃) δ: 1.25 (t, J=7.2 Hz, 3H), 3.60 (t, J=6.4 Hz, 2H), 3.80 (t, J=6.4 Hz, 2H), 4.20 (q, J=7.2 Hz, 2H).

### Step 2. 3,3,4,4-d₄-Tetrahydro-2H-pyran-2-one

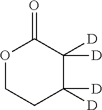

To a solution of ethyl 5-hydroxypentanoate-2,2,3,3-d4 (14.0 g, 93.3 mmol) in benzene (800 mL) was added anhydrous pTSA (p-toluenesulfonic acid) (10 mg) and the solution was heated to reflux using a Dean-Stark apparatus for 8 hours. The solution was cooled to room temperature and the reaction mixture was quenched with solid NaHCO₃. The reaction mixture was filtered and concentrated to give the crude product (10.0 g, 100%). ¹H NMR (400 MHz, CDCl₃): δ 1.80 (t, J=6.0 Hz, 2H), 4.20 (t, J=6.0 Hz, 2H)

### Step 3. 2,2,3,3-d₄-5-Bromopentanoic acid

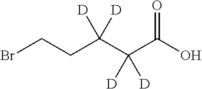

To a solution of BBr₃ (9.10 mL, 95.9 mmol) in methylene chloride (200 mL) was added δ-valerolactone-2,2,3,3-d4 (10.0 g, 95.9 mmol) in methylene chloride and the solution was stirred at room temperature overnight. The reaction mixture was quenched with D₂O (10 mL) and the solution was stirred at room temperature for 1 hour. The reaction mixture was mixed with water (50 mL) and was extracted with methylene chloride (2×25 mL), and the organic layer was dried over Na₂SO₄ and concentrated to give the titled product (3.50 g, 20%). ¹H NMR (400 MHz, CDCl₃): δ 1.90 (t, J=6.4 Hz, 2H), 3.40 (t, J=6.4 Hz, 2H).

### Step 4. (4-Carboxy-3,3,4,4-d₄-butyl)triphenylphosphonium bromide (17b) (Y^{1a/1b}=Y^{2a/2b}=D; Y^{3a/3b}=H)

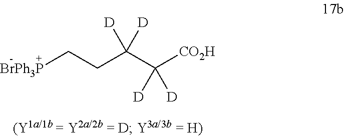

To a solution of 2,2,3,3-d₄-5-bromopentanoic acid (2.16 g, 11.72 mmol) in CD₃CN (23 mL), was added triphenylphosphine (3.07 g, 11.72 mmol, 1.0 equiv). The mixture was heated to reflux for a period of 15 hours then cooled to ambient temperature. The cooled solution was concentrated to one-half volume then diluted with Et₂O until a cloudy mixture was obtained. Crystallization was initiated by scratching the inside wall of the flask which resulted in the formation of a white precipitate. The mixture was filtered and the material washed with Et₂O. The pure material was lyophilized to remove trace solvents and afforded a white solid (2.76 g, 64%) of the titled product. MS (M+H): 367.1.

### Example 7

### Synthesis of (E)-2,2,3,3-d₄-5-((3aS,4R,5R,6aS)-5-Hydroxy-4-((S,E)-3-hydroxy-4-methyloct-1-en-6-ynyl)hexahydropentalen-2(1H)-ylidene)pentanoic acid (Compound 102)

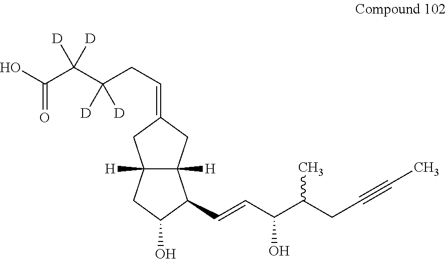

### Step 1. (E)-5-((3aS,4R,5R,6aS)-5-(tert-butyldimethylsilyloxy)-4-((S,E)-3-(tert-butyldimethylsilyloxy)-4-methyloct-1-en-6-ynyl)hexahydropentalen-2(1H)-ylidene)-2,2,3,3-tetradeuteropentanoic acid (19) (Y^{1a/1b}=Y^{2a/2b}=D; Y^{3a/3b}=Z^{1a/1b}=H)

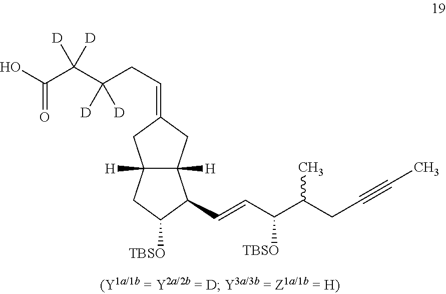

Two round-bottom flasks were flame-dried under nitrogen. One flask was charged with (4-carboxy-3,3,4,4-tetradeuterobutyl)triphenylphosphonium bromide 17b (815 mg, 1.82 mmol, see Example 6 for preparation) and then flushed with nitrogen. The compound was suspended in benzene to form a slurry, the flask was fitted onto a rotary evaporator, and the benzene was removed in vacuo. (Note that for this procedure, gas-tight plastic syringes were used to add all liquid reagents.) A nitrogen balloon was attached to the rotary evaporator and the flask was back-filled with nitrogen. The azeotrope procedure described below was repeated twice and then the flask was charged with a stir bar, placed in a vacuum desiccator containing Drierite and subjected to high vacuum for about 30 minutes. During this time, the second round-bottom bottom flask was charged with 18 (Z^{1a}=Z^{1b}=H) (181 mg, 0.358 mmol, see Example 4 for preparation) and then flushed with nitrogen. Compound 18 (Z^{1a}=Z^{1b}=H) was subjected to the azeotrope procedure described below three times, and then the flask was charged with a stir bar, placed in a vacuum desiccator and subjected to high vacuum for about 45 minutes. During this time the flask containing 17b was placed under nitrogen and dry THF (10.1 mL from a new bottle, ≧99.9%, inhibitor-free, Sigma-Aldrich) was added. To the resulting suspension was added t-BuOK (3.94 mL, 3.94 mmol, 1M solution in THF). The resulting red, cloudy mixture was stirred rapidly for 30 min. The flask containing Compound 18 (Z^{1a}=Z^{1b}=D) was placed under nitrogen. THF (3.59 mL) was added, and the resulting solution was added quickly via canula to the solution containing 17b. This was followed with a THF rinse (1.76 mL). After stirring overnight, the reaction was quenched with 50% citric acid in D₂O and diluted with ethyl acetate. The organic layer was washed twice with 50% citric acid in D₂O. The combined aqueous solutions were washed twice with ethyl acetate. The combined organic solutions were dried (Na₂SO₄), filtered and concentrated. Purification via automated flash column chromatography (40 g SiO₂, 12.5% EtOAc in heptanes) afforded 180 mg (85%) of titled product. ¹H NMR (300 MHz, C₆D₆) of mixture of diastereomers (only chemical shifts of major diastereomer are reported): δ 5.66 (m, 1H), 5.54 (m, 1H), 5.16 (br s, 1H), 4.20 (td, J=55.5, 5.2, 1H), 3.74 (q, J=8.1, 1H), 2.55-2.31 (m, 2H), 2.31-1.95 (m, 8H), 1.95-1.83 (m, 3H), 1.61 (s, 3H), 1.32 (m, 2H), 1.24-1.12 (m, 3H), 1.04 (s, 9H), 1.01 (s, 9H), 0.17 (s, 3H), 0.17 (s, 3H), 0.13 (s, 3H), 0.10 (s, 3H). MS (M−H): 590.9.

Azeotrope Procedure Used in Example 7, Step 1.

The material to be azeotroped was dissolved in benzene and the flask fitted onto a roto-evaporator (“rotovap”) (about 5 mL benzene per 180 mg of material.) The bath temperature was about 20° C. The initial pressure was set at 80 torr, and then the pressure was lowered in about 10 torr increments to a minimum of about 7 torr. A balloon filled with nitrogen was attached to an inlet valve on the rotavap, so that when the evaporation was complete the flask could be back-filled with nitrogen rather than air.

### Step 2. (E)-2,2,3,3-d₄-5-((3aS,4R,5R,6aS)-5-Hydroxy-4-((S,E)-3-hydroxy-4-methyloct-1-en-6-ynyl)hexahydropentalen-2(1H)-ylidene)pentanoic acid (Compound 102)

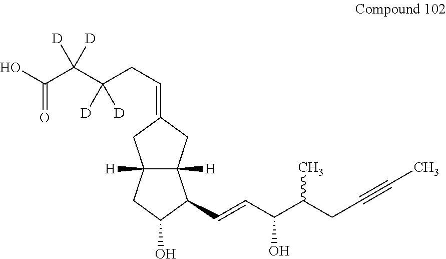

A 50-mL round-bottomed flask was charged with Compound 19 (Y^{1a}=Y^{1b}=Y^{2a}=Y^{2b}=D; Y^{3a}=Y^{3b}=Z^{1a}=Z^{1b}=H) (217 mg, 0.366 mmol) anhydrous THF (3.7 mL), and tetrabutylammonium fluoride (1.0 M solution in THF, 2.19 mL). The reaction mixture was stirred for 60 hours, then was diluted with ethyl acetate (5 mL), treated with D₂O (5 mL), and adjusted to pH=3 with 5% DCl in D₂O solution. The aqueous layer was extracted with ethyl acetate (3×25 mL) and the combined organic extracts were concentrated to an oil. The oil was purified by column chromatography on silica gel (10 cm×1.5 cm; eluent 5% MeOH in CH₂Cl₂) to deliver Compound 102 as a yellow oil and a portion of Compound 102 as a mixture of E/Z isomers (77 mg). The oil containing the desired isomer was dissolved in ethyl acetate (15 mL) and washed with 10% DCl in D₂O solution (3×3 mL) to deliver Compound 102 as a pale yellow oil (22 mg, 17%). The oil containing a mixture of E/Z isomers was dissolved in ethyl acetate (15 mL) and washed with 10% DCl in D₂O solution (3×3 mL). The organic layer was concentrated to an oil and purified by column chromatography on silica gel (10 cm×1.5 cm; eluent 3% MeOH in CH₂Cl₂) to deliver further Compound 102 as an oil (40 mg, 30% yield). HPLC (Column: Waters SunFire Prep Silica, 5 gni, 4.6×250 mm column; Mobile Phase: 96:4 hexanes/i-PrOH (1.5 mL/min); Wavelength: 254 nm): retention time: 12.32/13.06 minutes (Z) and 17.21/18.26 minutes (E); 89.6% purity (E). ¹H NMR (300 MHz, C₆D₆) of mixture of diastereomers (only chemical shifts of major diastereomer are reported): δ 5.70-5.61 (m, 1H), 5.54-5.41 (m, 1H), 5.16 (br s, 1H), 4.20 (dt, J₁=20.0, J₂=7.8, 1H), 3.67 (q, J=9.1, 1H), 2.46-1.90 (m, 12H), 1.58-1.56 (m, 3H), 1.32-0.88 (m, 7H). MS (M−H) 363.2.

### Example 8

### Synthesis of (E)-2,2,3,3-d₄-5-((3aS,4R,5R,6aS)-4-((S,E)-5,5-d₂-3-Hydroxy-4-methyloct-1-en-6-ynyl)-5-hydroxyhexahydropentalen-2(1H)-ylidene)pentanoic acid, (Compound 105)

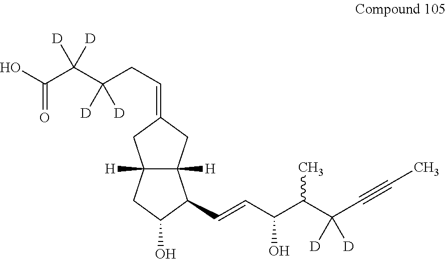

### Step 1. (E)-5-((3aS,4R,5R,6aS)-5-(tert-butyldimethylsilyloxy)-4-((S,E)-3-(tert-butyldimethylsilyloxy)-5,5-d₂-4-methyloct-1-en-6-ynyl)hexahydropentalen-2(1H)-ylidene)-2,2,3,3-d₄-pentanoic acid (19) (Y^{1a/1b}=Y^{2a/2b}=Z^{1a/1b}=D; Y^{3a/3b}=H)

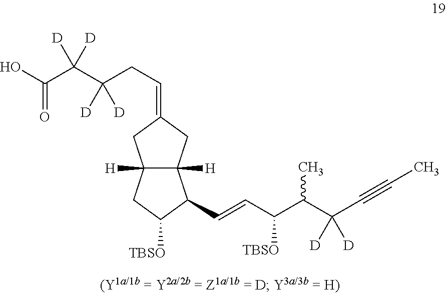

Two round-bottom flasks were flame-dried under nitrogen. One flask was charged with 17b (718 mg, 1.61 mmol, see Example 6 for preparation) and then flushed with nitrogen. The compound was suspended in benzene to form a slurry, the flask was fitted onto a rotary evaporator, and the benzene was removed in vacuo. (Note that for this procedure, gas-tight plastic syringes were used to add all liquid reagents.) A nitrogen balloon was attached to the rotary evaporator and the flask was back-filled with nitrogen. The azeotrope procedure described above was repeated twice and then the flask was charged with a stir bar, placed in a vacuum desiccator containing Drierite and subjected to high vacuum for approximately 30 min. During this time, the second round-bottom flask was charged with Compound 18 (Z^{1a}=Z^{1b}=D) (160 mg, 0.316 mmol, see Example 5 for preparation) and then flushed with nitrogen. Compound 18 (Z^{1a}=Z^{1b}=D) was subjected to the azeotrope procedure described above three times, and then the flask was charged with a stir bar, placed in the vacuum desiccator and subjected to high vacuum for approximately 45 min. During this time the flask containing 17b was placed under nitrogen and dry THF (8.91 mL from a new bottle, ≧99.9%, inhibitor-free, Sigma-Aldrich) was added. To the resulting suspension was added t-BuOK (3.48 mL, 3.48 mmol, 1M solution in THF from a recently-opened Sigma-Aldrich bottle, stored in a desiccator when not in use). The resulting red, cloudy mixture was stirred rapidly for 30 min. The flask containing Compound 18 (Z^{1a}=Z^{1b}=D) was placed under nitrogen. THF (3.16 mL) was added, and the resulting solution was added quickly via cannula to the solution containing 17b. This was followed with a THF rinse (1.61 mL). After stirring overnight, the reaction was quenched with 50% citric acid in D₂O and diluted with EtOAc. The organic layer was washed twice with 50% citric acid in D₂O. The combined aqueous solutions were washed twice with EtOAc. The combined organic solutions were dried (Na₂SO₄), filtered and concentrated. Purification on an ISCO instrument (40 g SiO₂, 12.5% EtOAc in heptanes) afforded 188 mg (quant.) of 19. ¹H NMR (300 MHz, C₆D₆) of mixture of diastereomers (only chemical shifts of major diastereomer are reported): δ 5.65 (m, 1H), 5.55 (m, 1H), 5.16 (br s, 1H), 4.20 (td, J=55.0, 4.9, 1H), 3.73 (q, J=8.2, 1H), 2.50-2.31 (m, 1H), 2.31-2.05 (m, 6H), 2.05-1.95 (m, 2H), 1.94-1.82 (m, 3H), 1.65 (s, 2H), 1.62-1.58 (m, 3H), 1.39-1.24 (m, 2H), 1.22-1.12 (m, 3H), 1.04 (s, 9H), 1.01 (s, 9H), 0.17 (s, 3H), 0.16 (s, 3H), 0.12 (s, 3H), 0.10 (s, 3H). MS (M+H): 594.7.

### Step 2. (E)-2,2,3,3-d₄-5-((3aS,4R,5R,6aS)-4-((S,E)-5,5-d₂-3-Hydroxy-4-methyloct-1-en-6-ynyl)-5-hydroxyhexahydropentalen-2(1H)-ylidene)pentanoic acid, (Compound 105)

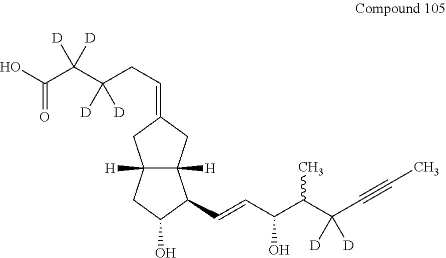

A 50-mL round-bottomed flask was charged with Compound 19 (Y^{1a}=Y^{1b}=Y^{2a}=Y^{2b}=Z^{1a}=Z^{1b}=D; Y^{3a}=Y^{3b}=H) (213 mg, 0.358 mmol) anhydrous THF (3.6 mL), and tetrabutylammonium fluoride (TBAF) (1.0 M solution in THF, 2.15 mL, 2.15 mmol). The reaction mixture was stirred for 48 hours with monitoring by TLC analysis. The reaction mixture was diluted with ethyl acetate (5 mL), treated with D₂O (5 mL), and adjusted to pH=3 with 5% DCl in D₂O solution. The aqueous layer was extracted with ethyl acetate (3×25 mL) and the combined organic extracts were concentrated to an oil. The oil was purified by column chromatography on silica gel (10 cm×1.5 cm; eluent 5% MeOH in CH₂Cl₂) to deliver Compound 105 as a yellow oil (96 mg, 73% yield) and a portion of Compound 105 as a mixture of E/Z isomers (86 mg). The oil containing the desired isomer was dissolved in ethyl acetate (15 mL) and washed with 10% DCl in D₂O solution (3×3 mL) to deliver Compound 105 as a pale yellow oil (13 mg, 10%): HPLC (Column: Waters SunFire Prep Silica, 5 μm, 4.6×250 mm column; Mobile Phase: 96:4 hexanes/1-PrOH (1.5 mL/min); Wavelength: 254 nm): retention time: 12.66/13.45 minutes (Z) and 17.79/18.92 minutes (E); 82.9% purity (E). ¹H NMR (300 MHz, C₆D₆) of mixture of diastereomers (only chemical shifts of major diastereomer are reported): δ 5.87-5.77 (m, 1H), 5.69-5.57 (m, 1H), 5.32 (br s, 1H), 4.30-4.19 (m, 1H), 3.87-3.79 (m, 1H), 2.50-2.02 (m, 10H), 1.74-1.72 (m, 3H), 1.47-1.23 (m, 7H). MS (M−H): 365.2.

The oil containing a mixture of E/Z isomers was dissolved in ethyl acetate (15 mL) and washed with 10% DCl in D₂O solution (3×3 mL). The organic layer was concentrated to an oil and purified by column chromatography on silica gel (10 cm×1.5 cm; eluent 5% MeOH in CH₂Cl₂). Purification delivered only mixed fractions, which were collected and concentrated to an oil. The oil was purified by column chromatography on silica gel (10 cm×1.5 cm; eluent 3% MeOH in CH₂Cl₂) to deliver Compound 102 as an oil (1 mg, 0.01%). Due to the small quantity of material, spectral data was not acquired for this material.

### Example 9

### Synthesis of (4-Carboxy-2,2,3,3-d₄-butyl)triphenylphosphonium bromide (17c) (Y^{1a/1b}=H; Y^{2a/2b}=Y^{3a/3b}=D)

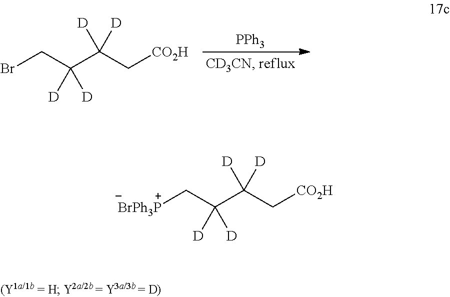

To a solution of commercially available 3,3,4,4-d₄-5-bromopentanoic acid (1.87 g, 10.1 mmol, 99.7% D) in CD₃CN (20 mL), was added triphenylphosphine (2.65 g, 10.1 mmol, 1.0 equiv). The mixture was heated to reflux for a period of 15 hours then cooled to ambient temperature. The cooled solution was concentrated to one-half volume then diluted with Et₂O until a cloudy mixture was obtained. Crystallization was initiated by scratching the inside wall of the flask which resulted in the formation of a white precipitate. The mixture was filtered and the material washed with Et₂O. The pure material was lyophilized to remove trace solvents and afforded a white solid (3.62 g, 80%) of 17c. MS: m/z 367.1 [M⁺].

### Example 10

### Synthesis of (E)-3,3,4,4-d₄-5-((3aS,4R,5R,6a5)-4-((S,E)-5,5-d₂-3-hydroxy-4-methyloct-1-en-6-ynyl)-5-hydroxyhexahydropentalen-2(1H)-ylidene)pentanoic acid (Compound 114)

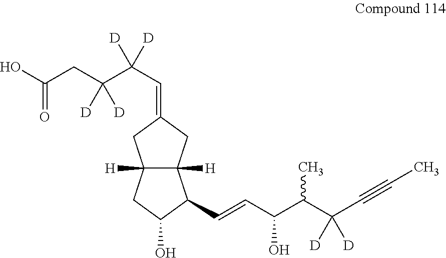

To a suspension of 17c (881 mg, 1.97 mmol, see Example 9 for preparation) in anhydrous THF (11 mL) was added KOt-Bu (1.0 M in THF, 4.34 mL, 4.34 mmol). The resultant orange solution was stirred at ambient temperature for 30 minutes followed by the addition of a solution of (18) (Z^{1a}=Z^{1b}=D) (200 mg, 0.395 mmol, see Example 5 for preparation) in THF (4 mL). The mixture was stirred at ambient temperature for 5 hours, then was quenched by the addition of saturated aqueous NH₄Cl solution (20 mL). The aqueous layer was extracted with EtOAc (3×25 mL) and the combined organic extracts were concentrated to an oil, which was purified by column chromatography on silica gel (10×3 cm; eluent 7:1 heptanes/EtOAc). The fractions containing product were separated into 2 portions and were concentrated to deliver 2 batches of product: 1) the product as a mixture of diastereomers (12 mg, 5% yield); and 2) the desired, more-polar compound with minor contamination by the undesired diastereomer (97 mg, 41% yield).

To a solution of the TBS ether of Compound 114 (97 mg, 0.163 mmol, batch 2 above) in anhydrous THF (1.6 mL) was added TBAF (1.0 M solution in THF, 978 μL, 0.978 mmol). The reaction mixture was stirred for 34 hours at ambient temperature, then the reaction was quenched by the addition of saturated aqueous NH₄Cl solution (5 mL). The mixture was diluted with water (5 mL), and was extracted with EtOAc (3×25 mL). The combined organic extracts were concentrated to an oil, and the resulting oil was purified by column chromatography on silica gel (10 cm×1.5 cm; eluent 5% MeOH in CH₂Cl₂) to deliver the desired isomer as a pale yellow oil (37 mg, 63% yield) and a portion of product as a mixture of diastereomers (16 mg, 27% yield).

A solution of the pure product above (37 mg) in EtOAc (10 mL) was washed with water (10 mL) and the pH of the aqueous layer was tested and found to be pH=6. The solution was washed with 5% aq HCl (0.5 mL) to adjust the pH of the aqueous layer to pH=3-4. The organic layer was removed and washed with two additional portions of the water (10 mL) and 5% aq HCl (0.5 mL) mixture. The organic layer was concentrated to deliver 12 mg of Compound 114 as an oil (12 mg, 20% yield). HPLC (Column: Waters SunFire Prep Silica, 5 μm, 4.6×250 mm column; Mobile Phase: 96:4 hexanes/i-PrOH (1.5 mL/min); Wavelength: 254 nm): retention time: 13.38/14.38 minutes (Z) and 18.42/19.61 minutes (E); 85.6% purity (E). ¹H NMR (300 MHz, C₆D₆) of mixture of diastereomers (only chemical shifts of major diastereomer are reported): δ 5.76-5.66 (m, 1H), 5.58-5.46 (m, 1H), 5.20 (br s, 1H), 4.19-4.08 (m, 1H), 3.76-3.68 (m, 1H), 2.38-1.89 (m, 10H), 1.62-1.60 (m, 3H), 1.35-0.92 (m, 7H). MS (M−H): 365.4.

### Example 11

### Synthesis of (E)-3,3,4,4-d₄-5-((3aS,4R,5R,6aS)-4-((S,E)-3-Hydroxy-4-methyloct-1-en-6-ynyl)-5-hydroxyhexahydropentalen-2(1H)-ylidene)pentanoic acid (Compound 111)

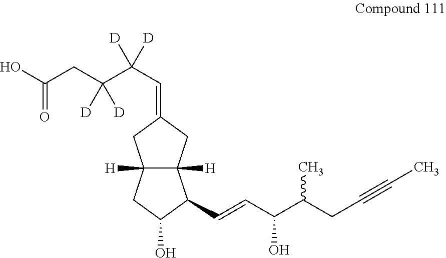

To a suspension of 17c (886 mg, 1.98 mmol, see Example 9 for preparation) in THF (11 mL) was added KOt-Bu (1.0 M solution in THF, 4.36 mL, 4.36 mmol). The mixture was stirred for 30 minutes at ambient temperature then was treated with (18) (Z^{1a}=Z^{1b}=H) (200 mg, 0.396 mmol, see Example 4 for preparation) in THF (4 mL). After 4 hours, the mixture was treated with NH₄Cl solution (15 mL) and adjusted to pH=4 with 10% aqueous citric acid solution. The aqueous layer was extracted with EtOAc (3×50 mL) and the combined organic extracts were concentrated to an oil and purified by column chromatography on silica gel (15 cm×1.5 cm; eluent 7:1 heptanes/EtOAc) to deliver the product as a yellow oil (137 mg, 59% yield).

A solution of the TBS ether of Compound 111 (137 mg, 0.231 mmol) in THF (2.5 mL was treated with TBAF (1.0 M solution in THF, 1.4 mL, 1.39 mmol) and stirred for 48 hours. The reaction mixture was diluted with EtOAc (5 mL), treated with saturated aqueous NH₄Cl solution (5 mL) and adjusted to pH=3 with 5% HCl. The aqueous layer was extracted with EtOAc (3×25 mL), and the combined organic extracts were washed with brine (50 mL) and concentrated to an oil. Purification of the resulting oil by column chromatography on silica gel (10×1.5 cm; eluent 5% MeOH in CH₂Cl₂) afforded Compound 111 as a pale yellow oil (16 mg, 20% yield). HPLC (Column: Waters SunFire Prep Silica, 5 μm, 4.6×250 mm column; Mobile Phase: 96:4 hexanes/i-PrOH (1.5 mL/min); Wavelength: 254 nm): retention time: 14.75/15.72 minutes (Z) and 20.74/21.99 minutes (E); 86.1% purity (E). ¹H NMR (300 MHz, C₆D₆) of mixture of diastereomers (only chemical shifts of major diastereomer are reported): δ 5.76-5.62 (m, 1H), 5.59-5.41 (m, 1H), 5.16 (br s, 1H), 4.38-4.05 (m, 1H), 3.70-3.65 (m, 1H), 2.41-1.90 (m, 12H), 1.58-1.56 (m, 3H), 1.30-1.08 (m, 6H), 0.96-0.82 (m, 1H). MS (M−H): 363.4.

### Example 12

### Synthesis of (E)-Isopropyl 3,3,4,4-d₄-5-((3aS,4R,5R,6aS)-4-((S,E)-3-Hydroxy-4-methyloct-1-en-6-ynyl)-5-hydroxyhexahydropentalen-2(1H)-ylidene)pentanoate (Compound 111b)

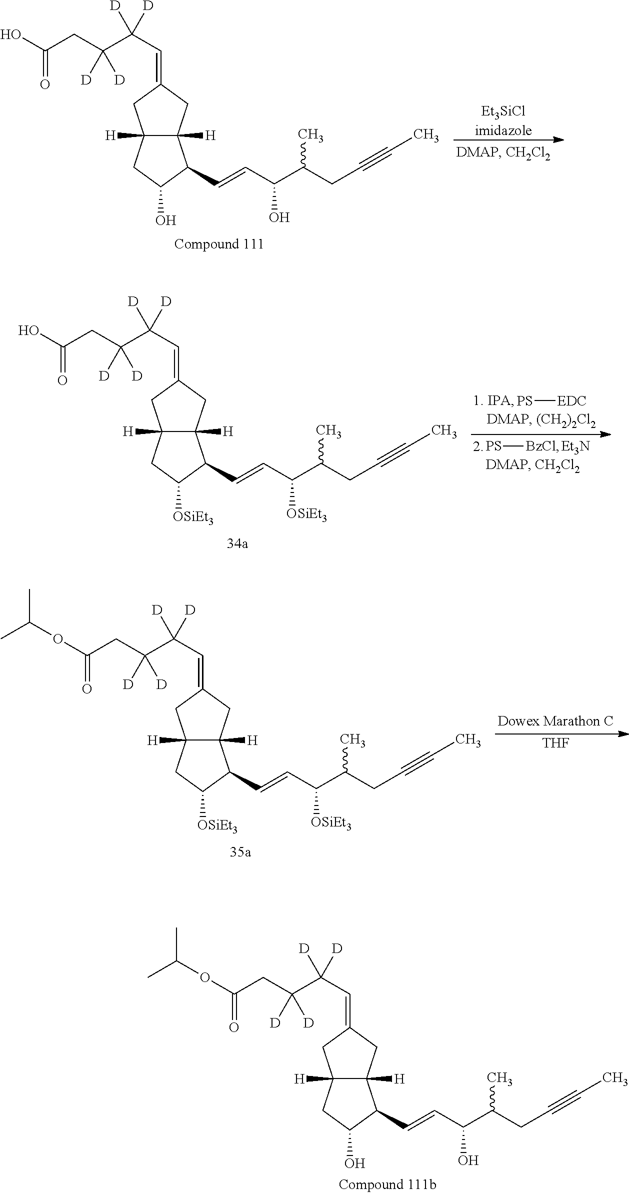

### Step 1. (E)-3,3,4,4-d₄-5-((3aS,4R,5R,6aS)-4-((S,E)-4-Methyl-3-(triethylsilyloxy)oct-1-en-6-ynyl)-5-(triethylsilyloxy)hexahydropentalen-2(1H)-ylidene)pentanoic acid (34a)

To a 0.2 M solution of 111 in CH₂Cl₂ at 0° C. is added imidazole (5 equiv) followed by Et₃SiCl (5 equiv) and DMAP (0.1 equiv). The reaction is stirred for 3 hours at 0° C. and allowed to warm to ambient temperature. After 12 hours at room temperature, the reaction is quenched with water (1 volume) and the phases are separated. The aqueous phase is extracted with CH₂Cl₂ and the combined organic phases are washed with brine, dried over Na₂SO₄, and concentrated under vacuum. The crude product is purified by column chromatography eluting with 20% EtOAc/heptanes to provide 34a.

### Step 2. (E)-Isopropyl-3,3,4,4-d₄-5-((3aS,4R,5R,6aS)-4-((S,E)-4-methyl-3-(triethylsilyloxy)oct-1-en-6-ynyl)-5-(triethylsilyloxy)hexahydropentalen-2(1H)-ylidene)pentanoic acid (35a)

To a vial containing 34a is added DCE. The solution is transferred with a pipette to a syringe with a filter frit (syringe 1) followed by a small DCE rinse. Syringe 1 is charged with a stir-bar, PS-EDC, DMAP, and isopropanol. Upon completion, the reaction solution is drained into a second syringe with a filter frit (syringe 2). Syringe 1 is rinsed with a small amount of DCE and the rinse is drained into syringe 2. The solution is concentrated to the desired volume under a stream of nitrogen. The solution is charged with a stir bar, Et₃N, DMAP, and benzoyl chloride on polystyrene. Upon completion, the mixture is poured onto a silica plug prepared by fitting a 100 mL pear-shaped flask with a 15 mL Buchner funnel and adding silica gel to the funnel. The plug is rinsed with CH₂Cl₂. The combined rinses are concentrated slightly under vacuum and transferred via pipette to a flask. The solution is concentrated nearly to dryness to afford Compound 111b.

### Step 3. (E)-Isopropyl 3,3,4,4-d₄-5-((3aS,4R,5R,6aS)-4-((S,E)-4-Methyl-3-(triethylsilyloxy)oct-1-en-6-ynyl)-5-(triethylsilyloxy)hexahydropentalen-2(1H)-ylidene)pentanoic acid (35a)

The flask containing 34a (from step 1) is charged with THF and Dowex resin. The solution is heated to 50° C. overnight. Upon completion, the solution is cooled to room temperature and poured onto a silica plug. The plug is rinsed with heptane, and the heptane rinse is discarded. The plug is then rinsed with 40% EtOAc in heptane. The combined rinses are concentrated slightly under vacuum and transferred via pipette to a vial. The solution is concentrated to dryness under a stream of nitrogen to afford Compound 111b.

### General Procedure for Examples 13-26

Scheme 5. General Procedure for the Synthesis of Compounds of Formula III (Examples 13-27).

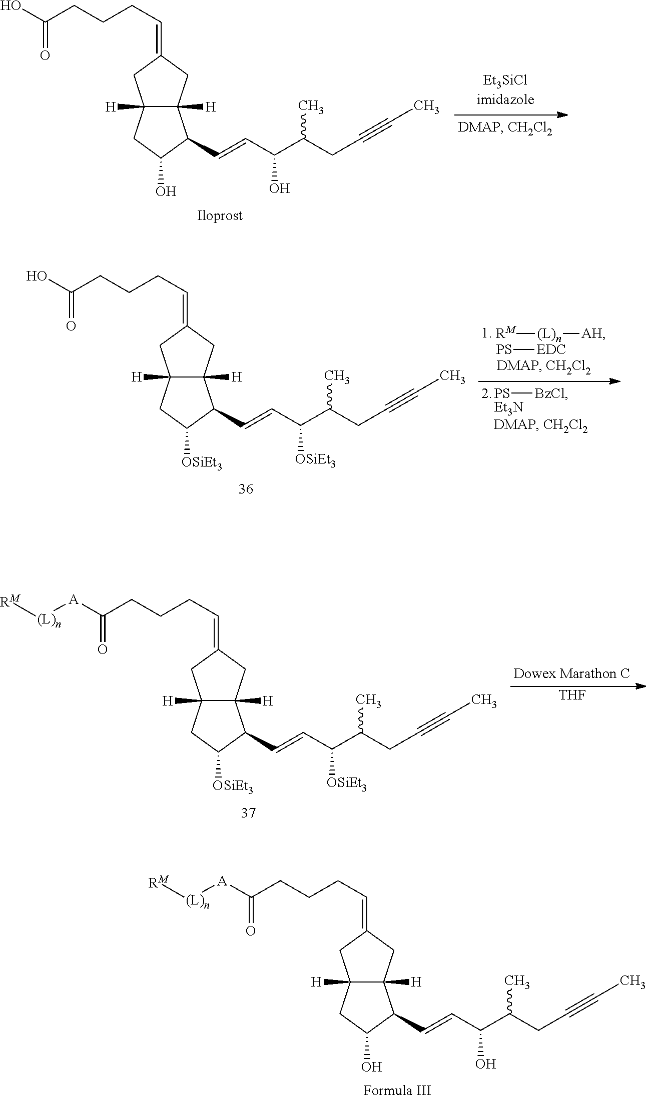

### Step 1. (E)-5-((3aS,4R,5R,6aS)-4-((3S,E)-4-methyl-3-((triethylsilyl)oxy)oct-1-en-6-yn-1-yl)-5-((triethylsilyl)oxy)hexahydropentalen-2(1H)-ylidene)pentanoic acid (36)

Commercially-available iloprost solution (5 mg/mL in methyl acetate, 40 mL, 0.56 mmol) was transferred to a 50 mL round-bottom flask and the solvent was removed under vacuum. CH₂Cl₂ (10 mL) was added followed by imidazole (189 mg, 5 equiv, 2.8 mmol), TESCl (0.464 mL, 5 equiv, 2.8 mmol), and DMAP (5 mg) at 0° C. TLC analysis after 15 h indicated that the reaction had reached completion. The reaction was quenched with aqueous HCl (0.01 N, 10 mL) and the phases were separated. The aqueous phase was extracted with CH₂Cl₂ (2×10 mL) and the combined organic extracts were washed with brine (10 mL), dried over Na₂SO₄, and concentrated under vacuum. The crude product was purified by column chromatography (silica gel: 30 g) eluting with 20% EtOAc/heptanes to provide 36 (203 mg, 63% yield. HRMS (M+Na): 611.3947.

### Step 2. 5-((3aS,4R,5R,6aS)-4-((3S,E)-4-methyl-3-((triethylsilyl)oxy)oct-1-en-6-yn-1-yl)-5-((triethylsilyl)oxy)hexahydropentalen-2(1H)-ylidene)pentanoate derivatives (37)

To a vial containing 37 (20 mg, 0.034 mmol) was added dichloroethane (1 mL). The solution is transferred with a pipette to a syringe with a filter frit (syringe 1), followed by a small dichloroethane rinse. Syringe 1 is charged with a stir-bar, PS-EDC (127 mg, ˜0.5 mmol/g, Sigma-Aldrich), DMAP (0.44 mg), and the alcohol (0.136 mmol). Upon completion, the reaction solution is drained into a second syringe with a filter frit (syringe 2). Syringe 1 is rinsed with a small amount of dichloroethane and the rinse is drained into syringe 2. The solution is concentrated to ˜1.8 mL under a stream of nitrogen. The solution is charged with a stir bar, Et₃N (0.0694 mL), DMAP (1.8 mg), and benzoyl chloride on polystyrene (304 mg, ˜0.9 mmol/g). After stirring overnight, the mixture is poured onto a silica plug. The plug is rinsed with CH₂Cl₂. The combined rinses are concentrated slightly under vacuum and transferred via pipette to a flask. The solution is concentrated nearly to dryness to afford 37.

### Step 3. 5-((3aS,4R,5R,6aS)-5-hydroxy-4-((3S,E)-3-hydroxy-4-methyloct-1-en-6-yn-1-yl)hexahydropentalen-2(1H)-ylidene)pentanoate derivatives (compounds of Formula III)

The flask containing 37 (from Step 2) is charged with THF (0.848 mL) and Dowex Marathon C resin (632 mg). The solution is heated to 50° C. overnight. Upon completion, the solution is cooled to room temperature and poured onto a silica plug. The plug is rinsed with heptane, and the heptane rinse is discarded. The plug is then rinsed with 40% EtOAc in heptane. The combined rinses are concentrated slightly under vacuum and transferred via pipette to a vial. The solution is concentrated to dryness under a stream of nitrogen to afford compounds of Formula III.

### Example 13

### Synthesis of (E)-Isobutyl 5-((3aS,4R,5R,6aS)-5-hydroxy-4-((3S,E)-3-hydroxy-4-methyloct-1-en-6-yn-1-yl)hexahydropentalen-2(1H)-ylidene)pentanoate (Compound 220)

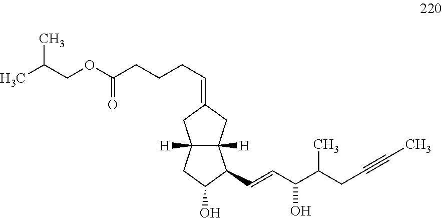

Compound 220 was prepared according to the General Procedure for the Synthesis of Compounds of Formula III. MS (M+H): 417.31.

### Example 14

### Synthesis of (E)-Heptyl 5-((3aS,4R,5R,6aS)-5-hydroxy-4-((3S,E)-3-hydroxy-4-methyloct-1-en-6-yn-1-yl)hexahydropentalen-2(1H)-ylidene)pentanoate (Compound 221)

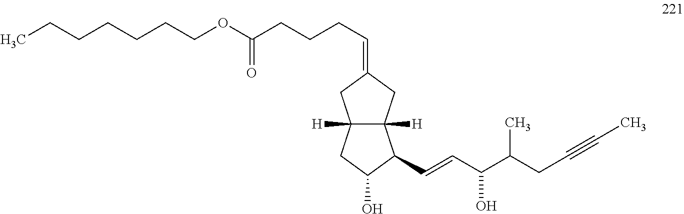

Compound 221 was prepared according to the General Procedure for the Synthesis of Compounds of Formula III. MS (M+H): 459.6.

### Example 15

### Synthesis of (E)-Isopropyl 5-((3aS,4R,5R,6aS)-5-hydroxy-4-((3S,E)-3-hydroxy-4-methyloct-1-en-6-yn-1-yl)hexahydropentalen-2(1H)-ylidene)pentanoate (Compound 222)

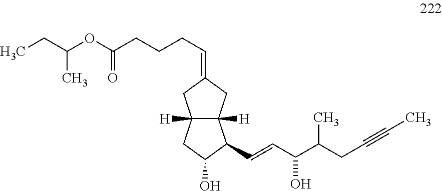

Compound 222 was prepared according to the General Procedure for the Synthesis of Compounds of Formula III. MS (M+H): 403.5.

### Example 16

### Synthesis of (E)-Neopentyl 5-((3aS,4R,5R,6aS)-5-hydroxy-4-((3S,E)-3-hydroxy-4-methyloct-1-en-6-yn-1-yl)hexahydropentalen-2(1H)-ylidene)pentanoate (Compound 223)

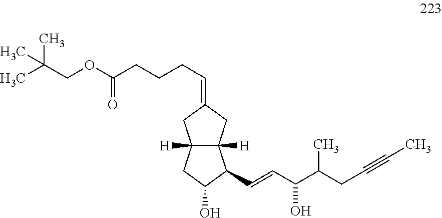

Compound 223 was prepared according to the General Procedure for the Synthesis of Compounds of Formula III. MS (M+H): 431.5.

### Example 17

### Synthesis of (E)-Cyclohexyl 5-((3aS,4R,5R,6aS)-5-hydroxy-4-((3S,E)-3-hydroxy-4-methyloct-1-en-6-yn-1-yl)hexahydropentalen-2(1H)-ylidene)pentanoate (Compound 224)

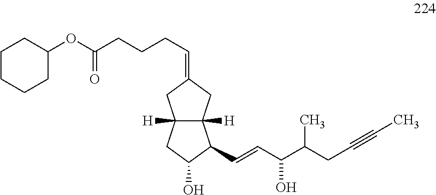

Compound 224 was prepared according to the General Procedure for the Synthesis of Compounds of Formula III. MS (M+H): 443.5.

### Example 18

### Synthesis of (E)-Benzyl 5-((3aS,4R,5R,6aS)-5-hydroxy-4-((3S,E)-3-hydroxy-4-methyloct-1-en-6-yn-1-yl)hexahydropentalen-2(1H)-ylidene)pentanoate (Compound 225)

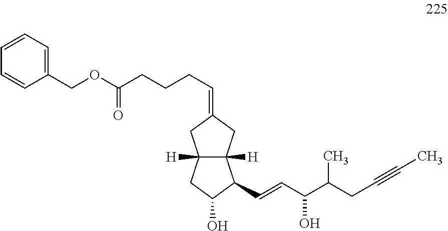

Compound 225 was prepared according to the General Procedure for the Synthesis of Compounds of Formula III. MS (M+H): 451.2.

### Example 19

### Synthesis of (E)-Benzhydryl 5-((3aS,4R,5R,6aS)-5-hydroxy-4-((3S,E)-3-hydroxy-4-methyloct-1-en-6-yn-1-yl)hexahydropentalen-2(1H)-ylidene)pentanoate (Compound 226)

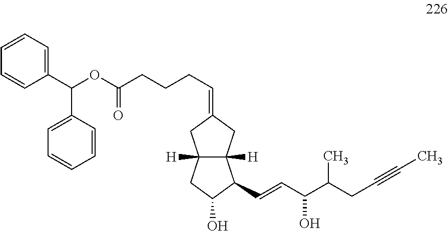

Compound 226 was prepared according to the General Procedure for the Synthesis of Compounds of Formula III. MS (M+H): 527.8.

### Example 20

### Synthesis of (E)-(S)-2-Ethoxy-2-oxo-1-phenylethyl 5-((3aS,4R,5R,6aS)-5-hydroxy-4-((3S,E)-3-hydroxy-4-methyloct-1-en-6-yn-1-yl)hexahydropentalen-2(1H)-ylidene)pentanoate (Compound 227)

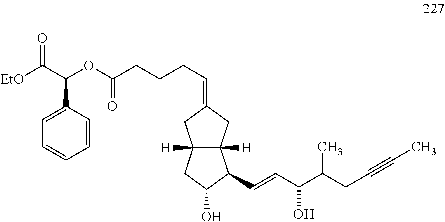

Compound 227 was prepared according to the General Procedure for the Synthesis of Compounds of Formula III. MS (M-OH): 505.7.

### Example 21

### Synthesis of (E)-(5-methyl-2-oxo-1,3-dioxol-4-yl)methyl 5-((3aS,4R,5R,6aS)-5-hydroxy-4-((3S,E)-3-hydroxy-4-methyloct-1-en-6-yn-1-yl)hexahydropentalen-2(1H)-ylidene)pentanoate (Compound 228)

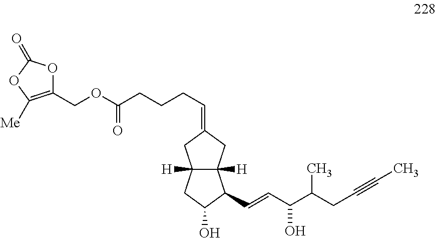

Compound 228 was prepared according to the General Procedure for the Synthesis of Compounds of Formula III. MS (M-OH): 455.1.

### Example 22

### Synthesis of (E)-2-(2-(2-Methoxyethoxy)ethoxy)ethyl 5-((3aS,4R,5R,6aS)-5-hydroxy-4-((3S,E)-3-hydroxy-4-methyloct-1-en-6-yn-1-yl)hexahydropentalen-2(1H)-ylidene)pentanoate (Compound 229)

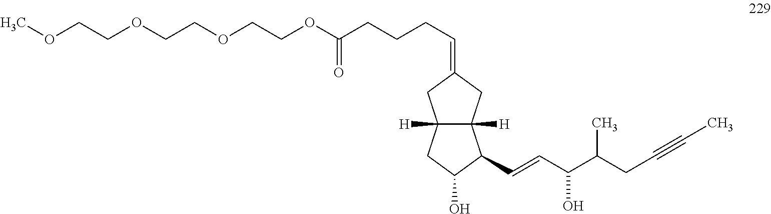

Compound 229 was prepared according to the General Procedure for the Synthesis of Compounds of Formula III. MS (M-OH): 489.8.

### Example 23

### Synthesis of (E)-(S)-Octan-2-yl 5-((3aS,4R,5R,6aS)-5-hydroxy-4-((3S,E)-3-hydroxy-4-methyloct-1-en-6-yn-1-yl)hexahydropentalen-2(1H)-ylidene)pentanoate (Compound 230)

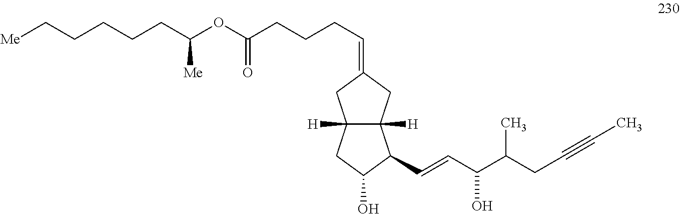

Compound 230 was prepared according to the General Procedure for the Synthesis of Compounds of Formula III. MS (M-OH): 455.1.

### Example 24

### Synthesis of (E)-(S)-1-Phenylethyl 5-((3aS,4R,5R,6aS)-5-hydroxy-4-((3S,E)-3-hydroxy-4-methyloct-1-en-6-yn-1-yl)hexahydropentalen-2(1H)-ylidene)pentanoate (Compound 231)

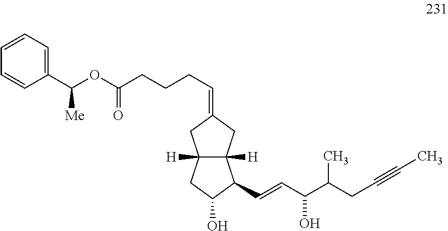

Compound 231 was prepared according to the General Procedure for the Synthesis of Compounds of Formula III. MS (M-OH): 447.2.

### Example 25

### Synthesis of (E)-(S)-3-Methylbutan-2-yl 5-((3aS,4R,5R,6aS)-5-hydroxy-4-((3S,E)-3-hydroxy-4-methyloct-1-en-6-yn-1-yl)hexahydropentalen-2(1H)-ylidene)pentanoate (Compound 232)

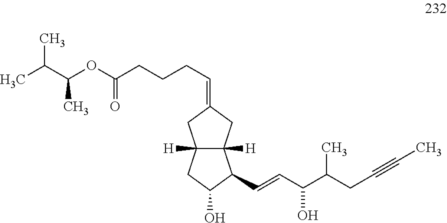

Compound 232 was prepared according to the General Procedure for the Synthesis of Compounds of Formula III. MS (M+H): 431.6.

### Example 26

### Synthesis of (2S)-Methyl 2-(((E)-5-((3aS,4R,5R,6aS)-5-hydroxy-4-((3S,E)-3-hydroxy-4-methyloct-1-en-6-yn-1-yl)hexahydropentalen-2(1H)-ylidene)pentanoyl)oxy)-4-methylpentanoate (Compound 233)

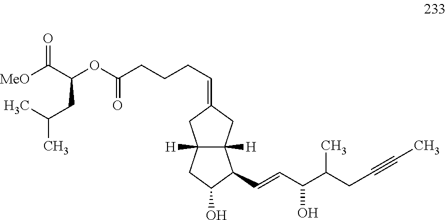

Compound 233 was prepared according to the General Procedure for the Synthesis of Compounds of Formula III. MS (M-OH): 471.2.

### Example 27

### Synthesis of (E)-(S)-1-methoxy-3-methyl-1-oxobutan-2-yl 5-((3aS,4R,5R,6aS)-5-hydroxy-4-((3S,E)-3-hydroxy-4-methyloct-1-en-6-yn-1-yl)hexahydropentalen-2(1H)-ylidene)pentanoate (Compound 237)

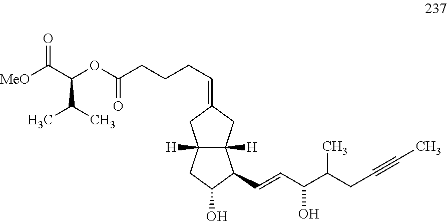

Compound 237 was prepared according to the General Procedure for the Synthesis of Compounds of Formula III. MS (M+H): 475.4.

### Example 28

### Synthesis of (E)-5-((3aS,4R,5R,6aS)-5-hydroxy-4-((3S,E)-3-hydroxy-4-methyloct-1-en-6-yn-1-yl)hexahydropentalen-2(1H)-ylidene)-N-(methylsulfonyl)pentanamide (Compound 234)

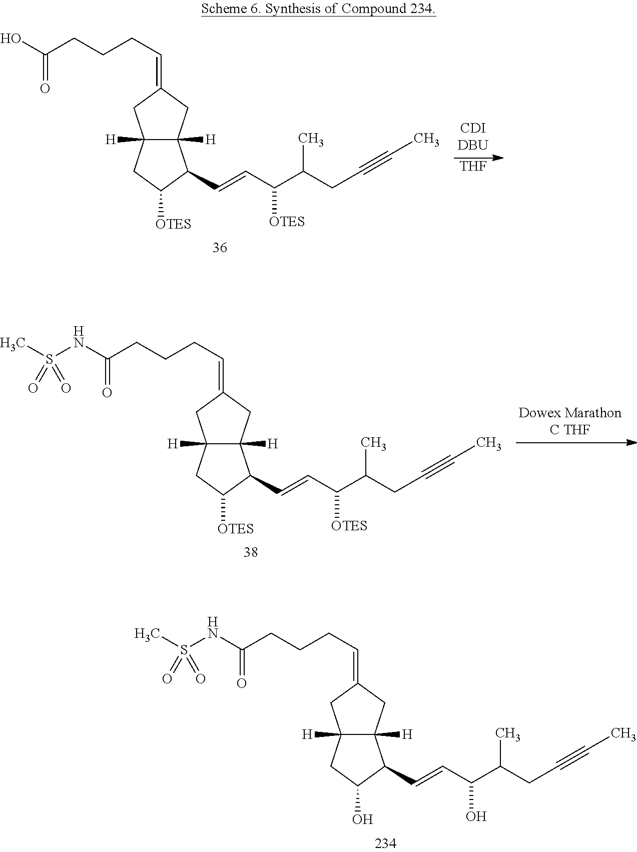

To a solution of 36 (20 mg, 0.0340 mmol) in THF (0.14 mL) was added 1,1′-carbonyldiimidazole (6.1 mg, 0.0374 mmol). The reaction was stirred overnight, whereupon DBU (0.0051 mL, 0.0340 mmol) and methanesulfonamide (3.6 mg, 0.0374 mmol) were added. The reaction was stirred at room temperature for 66 h, and then heated at 50° C. for 3 hours. The reaction was cooled to room temperature, and then quenched with pH 7 buffer. The aqueous solution was extracted with EtOAc (3×). The combined organic solutions were dried (Na₂SO₄), filtered and concentrated. Purification on an ISCO instrument (4 g SiO₂, 0 to 10% MeOH in CH₂Cl₂) afforded 38. 38 was dissolved in THF (0.848 mL) and Dowex Marathon C (632 mg) was added. The solution was heated to 50° C. overnight. Upon completion, the solution was cooled to room temperature and poured onto a silica plug. The plug was rinsed with heptane, and the heptane rinse is discarded. The plug was then rinsed with 10% MeOH in CH₂Cl₂. The combined rinses were concentrated slightly under vacuum and transferred via pipette to a vial. The solution was concentrated to dryness under a stream of nitrogen to afford 234. LCMS (M−H): 436.0.

### Example 29

### Synthesis of (E)-(R)-1-Ethoxy-1-oxopropan-2-yl 5-((3aS,4R,5R,6aS)-5-hydroxy-4-((3S,E)-3-hydroxy-4-methyloct-1-en-6-yn-1-yl)hexahydropentalen-2(1H)-ylidene)pentanoate (Compound 235)

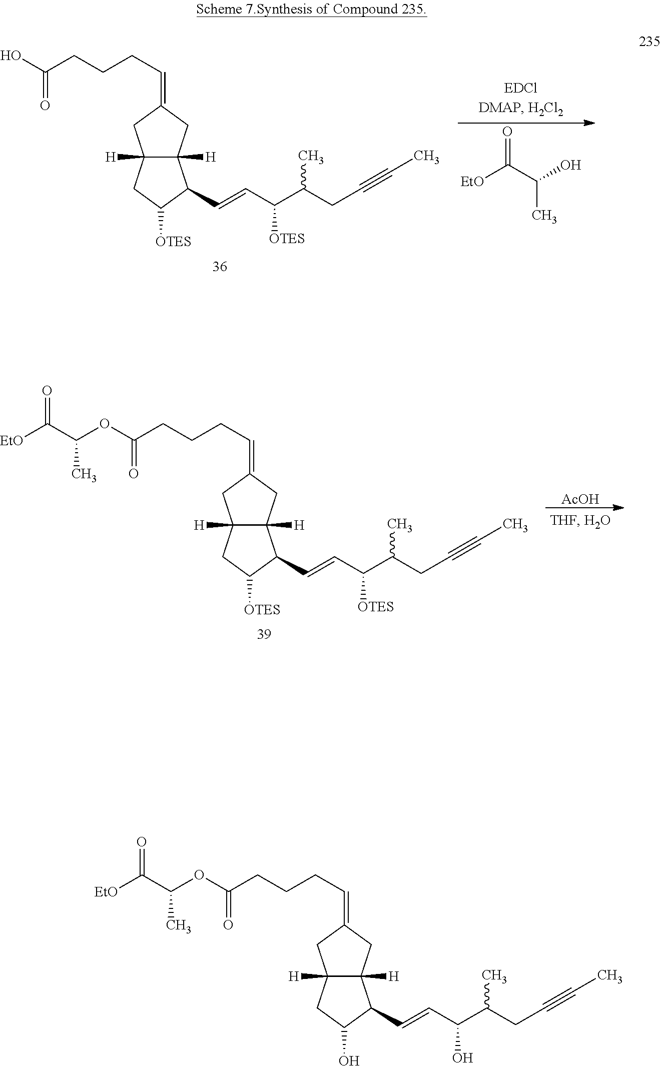

To a solution of 36 (70 mg, 0.117 mmol) in CH₂Cl₂ (3 mL) was added EDCI (49.3 mg, 0.254 mmol, 2.2 equiv), Et₃N (0.019 mL, 0.254 mmol, 2.2 equiv) and DMAP (3 mg) followed by ethyl L-lactate (0.11 mL, 0.936 mmol, 8 equiv). Formation of 39 was complete after 14 hours. The crude was concentrated and purified by silica-gel chromatography eluting with 15% EtOAc/heptanes to provide 39 as an oil (40 mg, 50% yield). This material was used for the synthesis of compound 235.

THF (6 mL) and water (2 mL) were added to the above solution of 39. The solution was stirred and AcOH (0.8 mL) was added. The reaction was stirred for 12 hours. Brine (10 mL) was added and the organic layer was extracted with ethyl acetate (10 mL). The combined organic extracts were washed with brine (3×5 mL), dried over Na₂SO₄ and concentrated. The crude was purified by silica-gel chromatography eluting with 50% EtOAc/heptanes to provide 235 as an oil (17 mg, 58% yield): HPLC (method: Advanced Materials Technology Halo 4.6×150 mm, 2.7 micron—gradient method: 1) 40% ACN+0.05% TFA for 10 min, then 2) 90% ACN+0.05% TFA for 30 min then 3) 100% ACN+0.05% TFA for 30 min then 4) 40% ACN+0.05% TFA for 36 min; wavelength: 210 nm): retention time: 22.34 min and 22.67 min; 97.3% purity. HRMS (M+Na): 483.2714.

### Example 30

### Synthesis of (E)-(R)-1-Ethoxy-1-oxopropan-2-yl 5-((3aS,4R,5R,6aS)-5-acetoxy-4-((3S,E)-3-acetoxy-4-methyloct-1-en-6-yn-1-yl)hexahydropentalen-2(1H)-ylidene)pentanoate (Compound 236)

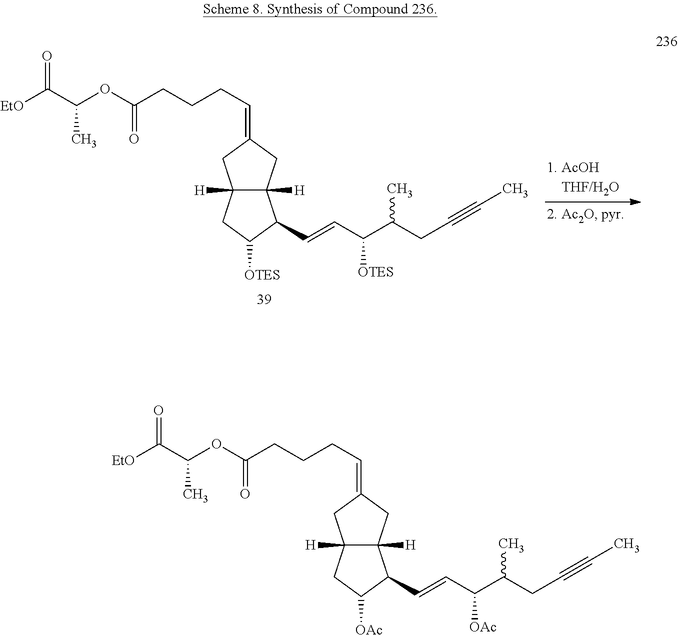

To a solution of 39 (40 mg, 0.056 mmol) in THF (6 mL) and water (2 mL) was added AcOH (0.8 mL). The reaction was stirred at ambient temperature for 14 hours. Brine (10 mL) was added, and the mixture was extracted with ethyl acetate (10 mL). The combined organic extracts were washed with brine (3×5 mL), dried over Na₂SO₄ and concentrated. The crude was dissolved in CH₂Cl₂, filtered and concentrated. The resulting oil was combined with the two crude reactions from previous small-scale runs, and pyridine (5 mL), Ac₂O (2.5 mL), and DMAP (2 mg) were added. The reaction was stirred for 15 h, at which point TLC (3:1 heptanes/EtOAc, R_{f} 0.35) indicated the reaction reached completion. The reaction was quenched with satd NH₄Cl in water (10 mL) and was extracted with Et₂O (3×20 mL). The combined organic extracts were washed sequentially with aqueous HCl (1 N, 10 mL), satd NaHCO₃ (10 mL) and brine (10 mL) and dried over Na₂SO₄. The extracts were filtered and the filtrate was concentrated. The crude was purified by flash-column chromatography (SiO₂: 8 g) eluting with 15% EtOAc/heptanes. ¹H NMR analysis of the resulting pure fractions indicated that a trace of an unknown impurity was present. Repurification by flash-column chromatography gave 236 as an oil (19 mg, 70% yield). HPLC analysis indicated a purity of 83% (AUC). This material was repurified by flash-column chromatography and the fractions were analyzed by HPLC. The fractions contained >96% (AUC) of the desired product and were combined, concentrated and dried under vacuum to provide 236 as a colorless oil (15 mg). HPLC (method: Advanced Materials Technology Halo 4.6×150 mm, 2.7 micron—gradient method: 1) 40% ACN+0.05% TFA for 10 min, then 2) 90% ACN+0.05% TFA for 30 min then 3) 100% ACN+0.05% TFA for 30 min then 4) 40% ACN+0.05% TFA for 36 min; wavelength: 210 nm): retention time: 29.30 min; 98.2% purity; HRMS (M+Na): 567.2929.

### Example 31

### Evaluation of Microsomal Stability

For the compounds of the invention shown in Table 6 below, the stability in rat intestinal microsomes (RIM) and human intestinal microsomes (HIM) was determined as follows. The compound (25 μM conc) was incubated with RIM or HIM (1 mg/mL) for 30 minutes at 37° C. For each compound, the amount of iloprost produced in 30 minutes was measured by LC-MS/MS, and the concentration was determined from a standard curve. The percentage of compound of the invention converted to iloprost was then calculated.

In Table 6, the difference in the observed percentage of compound of the invention converted to iloprost between run 1 and run 2 (both for RIM and for HIM) is believed to be affected by the time for which the samples were stored prior to the run—degradation of the analyte iloprost is believed to occur over time.

TABLE 6
Percentage of compound of the invention converted to iloprost

| | | RIM | | HIM | |
|---|---|---|---|---|---|
| Com- | Purity | % converted to Iloprost | | % converted to Iloprost | |
| pound | (%) | Run 1 | Run 2 | Run 1 | Run 2 |
| 236 | 99.5 | 2.16 | 0.002 | 0 | 0 |
| 232 | 81.0 | 3.56 | 3.94 | 1.82 | 0.52 |
| 235 | 97.3 | 7.65 | 0.76 | 1.54 | 0.48 |
| 233 | 71.0 | 9.11 | 6.50 | 3.46 | 1.12 |
| 230 | 73.0 | 9.21 | 7.34 | 2.10 | 0.55 |
| 224 | 86.0 | 13.14 | 7.95 | 1.99 | 0.71 |
| 221 | 86.0 | 14.67 | 19.8 | 6.67 | 2.42 |
| 226 | 79.0 | 17.04 | 25.2 | 1.03 | 0.30 |
| 237 | 78.0 | 18.07 | 12.6 | 6.10 | 2.13 |
| 227 | 78.0 | 18.42 | 3.34 | 2.10 | 0.76 |
| 222 | 80.0 | 19.09 | 1.95 | 4.28 | 1.20 |
| 231 | 82.0 | 19.71 | 10.4 | 0.96 | 0.23 |
| 223 | 78.0 | 21.41 | 12.7 | 1.86 | 1.25 |
| 229 | 92.0 | 35.02 | 4.89 | 7.37 | 2.59 |
| 220 | 80.0 | 64.73 | 14.6 | 8.84 | 2.48 |
| 225 | 81.0 | 89.82 | 21.9 | 11.51 | 3.76 |

Without further description, it is believed that one of ordinary skill in the art can, using the preceding description and the illustrative examples, make and utilize the compounds of the present invention and practice the claimed methods. It should be understood that the foregoing discussion and examples merely present a detailed description of certain preferred embodiments. It will be apparent to those of ordinary skill in the art that various modifications and equivalents can be made without departing from the spirit and scope of the invention.

We claim:

1. A compound of Formula I: 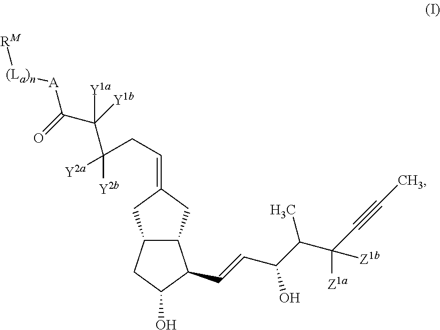
or a pharmaceutically acceptable salt thereof, wherein:
each Y is independently selected from hydrogen and deuterium;
each Z is independently selected from hydrogen, deuterium and fluorine;
wherein if Z^{1a} and Z^{1b} are each hydrogen then at least one Y is deuterium; 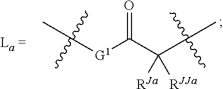
n=0 or 1;
R^{Ja} is C₁-C₂₀ alkyl or C₆-C₁₄ aryl, wherein R^{Ja} is optionally substituted with one or more J;
each J is independently halogen, C₁-C₆ alkyl, hydroxyl, O—(C₁-C₆ alkyl), NH₂, NH(C₁-C₆ alkyl), N(C₁-C₆ alkyl)₂, NHC(═NH)NH₂, SH, SCH₃, CONH₂, COOH, CO₂(C₁-C₆ alkyl), C₆-C₁₀ aryl, or 5-14-membered heteroaryl,
provided that if R^{Ja} is C₁-C₂₀ alkyl then J is not halogen;
R^{JJa} is H or C₁-C₆ alkyl;
or R^{Ja} and R^{JJa}, taken together with the carbon to which they are both attached, form a C₃-C₁₀ carbocyclic ring;
R^{M} is C₁-C₂₀ linear alkyl, C₁-C₂₀ branched alkyl, C₆-C₁₄ aryl, 5-14-membered heteroaryl, C₃-C₁₄ cycloalkyl, or 3-14-membered heterocycloalkyl, wherein R^{M} is optionally substituted with one or more M;
each M is independently C₁-C₆ alkyl, C₆-C₁₀ aryl, or heterocycloalkyl optionally substituted with one or more W, wherein each W is independently C₁-C₆ alkyl or ═O;
wherein if R^{M} is C₁-C₂₀ linear alkyl, then one or more methylene groups in R^{M} are optionally replaced with oxygen, with the proviso that each oxygen is separated from any other oxygen by at least two methylene groups;
A is —O—, —NH—, or —NH—SO₂—,
with the proviso that, if A is —NH—SO₂—, then n is 0, R^{M} is bound to the —SO₂— of A, and R^{M} is C₁-C₂₀ branched alkyl optionally substituted with halogen, C₁-C₂₀ linear alkyl optionally substituted with halogen, C₆-C₁₀ aryl, 5-10-membered heteroaryl, or 3-10-membered heterocycloalkyl, wherein in the R^{M} C₁-C₂₀ linear alkyl no methylene group is replaced with oxygen; and
G¹ is O, NH or CH₂.

2. The compound of claim 1, wherein A is —O—, n is 0, and R^{M} is C₁-C₂₀ linear alkyl or C₁-C₂₀ branched alkyl wherein R^{M} is optionally substituted with one or more M.

3. The compound of claim 2, wherein R^{M} is methyl, ethyl, isopropyl, isobutyl, neopentyl, n-heptyl, 2-octyl, 3-methyl-1-butyl, or 3-methyl-2-butyl, each optionally substituted with one or more M.

4. The compound of claim 2, wherein each of the one or more M is phenyl.

5. (canceled)

6. (canceled)

7. The compound of claim 2, wherein each of the one or more M is 3-14-membered heterocycloalkyl optionally substituted with one or more W.

8. The compound of claim 7, wherein R^{M} is methyl and M is 4-methyl-1,3-dioxol-2-onyl.

9. The compound of claim 1 wherein A is —O—, n is 0, and R^{M} is C₆-C₁₄ aryl, C₃-C₁₄ cycloalkyl, or 3 to 14-membered heterocycloalkyl, wherein R^{M} is optionally substituted with one or more M.

10. The compound of claim 9, wherein R^{M} is cyclohexyl, phenyl optionally substituted with one or more methyl or quinuclidinyl.

11. (canceled)

12. (canceled)

13. The compound of claim 1, wherein A is —O—, n is 0, and R^{M} is C₁-C₂₀ linear alkyl wherein one or more methylene groups in R^{M} are replaced with oxygen.

14. (canceled)

15. The compound of claim 1, wherein A is —NH—SO₂— and R^{M} is C₁-C₆ branched alkyl optionally substituted with one or more halogen, C₁-C₆ linear alkyl optionally substituted with one or more halogen, phenyl, 5-10-membered heteroaryl, or 3-10-membered heterocycloalkyl.

16. The compound of claim 15, wherein R^{M} is methyl optionally substituted with one or more fluorine, isopropyl, phenyl, 2-thiophenyl, 3-thiophenyl, or N-morpholinyl.

17. The compound of claim 1, wherein A is —O— or —NH— and n is 1.

18. The compound of claim 17, wherein R^{M} is C₁-C₂₀ linear alkyl or C₁-C₂₀ branched alkyl wherein R^{M} is optionally substituted with one or more M.

19. (canceled)

20. (canceled)

21. (canceled)

22. (canceled)

23. (canceled)

24. (canceled)

25. The compound of claim 1, wherein Y^{1a} and Y^{1b} are simultaneously deuterium.

26. (canceled)

27. The compound of claim 1, wherein Y^{2a} and Y^{2b} are simultaneously deuterium.

28. The compound of herein Z^{1a} and Z^{1b} are independently selected from deuterium or fluorine.

29. (canceled)

30. The compound of claim 1, wherein Z^{1a} and Z^{1b} are simultaneously deuterium or simultaneously fluorine.

31. (canceled)

32. The compound of claim 1, wherein 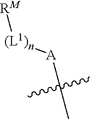
is selected from the group consisting of the following:

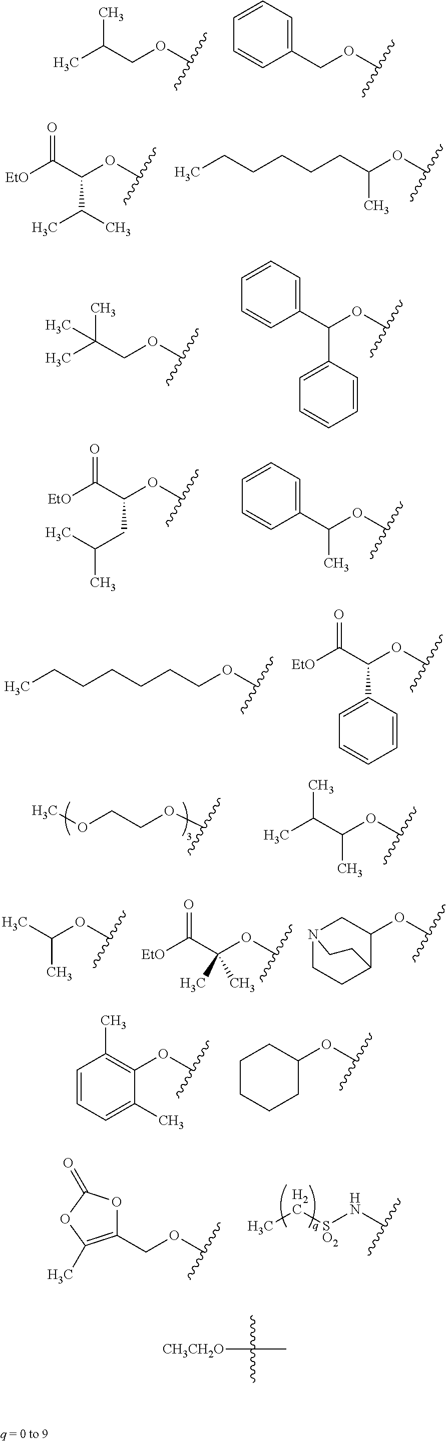

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. The compound of claim 1, or a pharmaceutically acceptable salt, wherein R^{M}-(Lₐ)ₙ-A- is 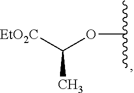
(CH₃)₂CHO—, or CH₃CH₂O—, and Y^{1a}, Y^{1b}, Y^{2a}, Y^{2b}, Z^{1a} and Z^{1b} are as set forth in the following table:

| Y^{1a} | Y^{1b} | Y^{2a} | Y^{2b} | Z^{1a} | Z^{1b} |
|---|---|---|---|---|---|
| D | D | H | H | H | H |
| H | H | D | D | H | H |
| D | D | D | D | H | H |
| D | D | H | H | D | D |
| H | H | D | D | D | D |
| D | D | D | D | D | D |
| D | D | H | H | F | F |
| H | H | D | D | F | F |
| D | D | D | D | F | F |
| H | H | H | H | D | D |

63. The compound of claim 1, or a pharmaceutically acceptable salt wherein R^{M}-(Lₐ)ₙ-A- is 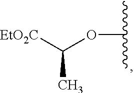
(CH₃)₂CHO—, or CH₃CH₂O—, and Y^{1a}, Y^{1b}, Y^{2a}, Y^{2b}, Y^{3a}, Y^{3b}, Z^{1a} and Z^{1b} are as set forth in the following table:

| Y^{1a} | Y^{1b} | Y^{2a} | Y^{2b} | Y^{3a} | Y^{3b} | Z^{1a} | Z^{1b} |
|---|---|---|---|---|---|---|---|
| D | D | H | H | D | D | H | H |
| H | H | D | D | D | D | H | H |
| D | D | D | D | D | D | H | H |
| D | D | H | H | D | D | D | D |
| H | H | D | D | D | D | D | D |
| D | D | D | D | D | D | D | D |

64. A compound of Formula A: 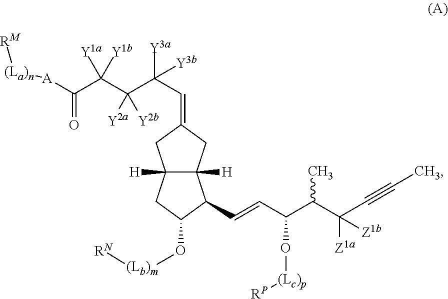
or a pharmaceutically acceptable salt thereof, wherein:
each Y is independently selected from hydrogen and deuterium;
each Z is independently selected from hydrogen, deuterium and fluorine;
Lₐ is 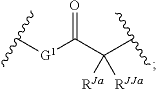
n=0 or 1;
R^{Ja} is C₁-C₂₀ alkyl or C₆-C₁₄ aryl, wherein R^{Ja} is optionally substituted with one or more J;
R^{JJa} is H or C₁-C₆ alkyl; or
R^{Ja} and R^{JJa}, taken together with the carbon atom to which they are both attached, form a C₃-C₁₀ carbocyclic ring;
L_{b} is 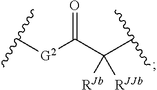
m=0 or 1;
R^{Jb} is C₁-C₂₀ alkyl or C₆-C₁₄ aryl, wherein R^{Jb} is optionally substituted with one or more J;
R^{JJb} is H or C₁-C₆ alkyl; or
R^{Jb} and R^{JJb}, taken together with the carbon atom to which they are both attached, form a C₃-C₁₀ carbocyclic ring;
L_{c} is 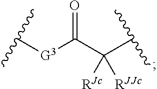
p=0 or 1;
R^{Jc} is C₁-C₂₀ alkyl or C₆-C₁₄ aryl, wherein R^{Jc} is optionally substituted with one or more J;
R^{JJc} is H or C₁-C₆ alkyl; or
R^{Jc} and R^{JJc}, taken together with the carbon atom to which they are both attached, form a C₃-C₁₀ carbocyclic ring;
each J is independently halogen, C₁-C₆ alkyl, hydroxyl, O—(C₁-C₆ alkyl), NH₂, NH(C₁-C₆ alkyl), N(C₁-C₆ alkyl)₂, NHC(═NH)NH₂, SH, SCH₃, CONH₂, COOH, CO₂(C₁-C₆ alkyl), C₆-C₁₀ aryl, or 5-14-membered heteroaryl, provided that if R^{J} is C₁-C₂₀ alkyl then J is not halogen;
R^{M} is C₁-C₂₀ linear alkyl, C₁-C₂₀ branched alkyl, C₆-C₁₄ aryl, 5-14-membered heteroaryl, C₃-C₁₄ cycloalkyl, or 3-14-membered heterocycloalkyl, wherein R^{M}, R^{N} and R^{P} are each optionally substituted with one or more M;
R^{N} and R^{P} are each independently selected from H, (CO)_{z}—C₁-C₂₀ linear alkyl, (CO)_{z}—C₁-C₂₀ branched alkyl, C₆-C₁₄ aryl, 5-14-membered heteroaryl, C₃-C₁₄ cycloalkyl, or 3-14-membered heterocycloalkyl, wherein when R^{N} is not H, R^{N} is optionally substituted with one or more M; and wherein when R^{P} is not H, R^{P} is optionally substituted with one or more M;
z is 0 or 1;
each M is independently C₁-C₆ alkyl, C₆-C₁₀ aryl, or 3-14-membered heterocycloalkyl wherein the M alkyl, aryl and/or heterocycloalkyl is optionally substituted with one or more W, wherein each W is independently C₁-C₆ alkyl or ═O, wherein if R^{M}, R^{N} or R^{P} is C₁-C₂₀ linear alkyl, then one or more methylene groups in R^{M}, R^{N} or R^{P} are optionally replaced with oxygen, with the proviso that each oxygen replacement is separated from any other oxygen replacement by at least two methylene groups;
A is —O—, —NH—, or —NH—SO₂—, with the proviso that, if A is —NH—SO₂—, then n is 0, R^{M} is bound to the —SO₂— of A, and R^{M} is C₁-C₂₀ branched alkyl optionally substituted with halogen, C₁-C₂₀ linear alkyl optionally substituted with halogen, C₆-C₁₀ aryl, 5-10-membered heteroaryl, or 3-10-membered heterocycloalkyl, wherein in the R^{M} C₁-C₂₀ linear alkyl no methylene group is replaced with oxygen; and
G¹, G² and G³ are each independently selected from O, NH and CH₂.

65. A compound of claim 64 or a pharmaceutically acceptable salt thereof, wherein the compound is a compound of Formula IV:

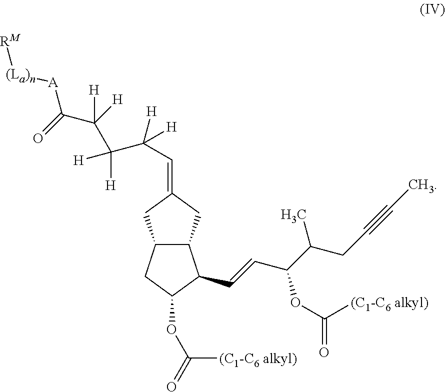

66. The compound of claim 65 set forth below: 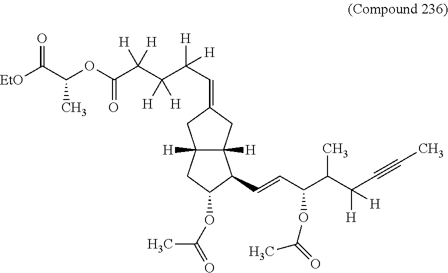
or a pharmaceutically acceptable salt thereof.

67. The compound of 1, wherein any atom not designated as deuterium is present at its natural isotopic abundance.

68. A pyrogen-free composition comprising an effective amount of a compound according 1 and a pharmaceutically acceptable carrier.

69. (canceled)

70. (canceled)

71. (canceled)

72. (canceled)

73. (canceled)

74. (canceled)

75. (canceled)

76. (canceled)

77. (canceled)

78. A method of treating a subject suffering from or susceptible to a disease or condition selected from pulmonary arterial hypertension, Raynaud's phenomenon secondary to systemic sclerosis, contrast-mediated nephropathy, and lung cancer comprising the step of administering to the subject in need thereof a composition of claim 64.

79. The method according to claim 78, wherein said disease is pulmonary arterial hypertension.

80. (canceled)

81. (canceled)

82. (canceled)

83. (canceled)
